SEQUENCE LISTING (10 sequences)

SEQ ID NO: 1 - protein, 1368 aa, Artificial Sequence
  Synthetic
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 2 - RNA, 86 nt, Artificial Sequence
  Synthetic
ggaaccauucaaaacagcauagcaaguuuaaauaaggcuaguccguuaucaacuugaaaaaguggcaccgagucggugcuuuuuuu

SEQ ID NO: 3 - RNA, 33 nt, Artificial Sequence
  Synthetic
ugauaucacagguuuaagagcuaugcuguuuug

SEQ ID NO: 4 - RNA, 33 nt, Artificial Sequence
  Synthetic
accauuccuucguuuaagagcuaugcuguuuug

SEQ ID NO: 5 - RNA, 115 nt, Artificial Sequence
  Synthetic
guuaggguuaggguuaggguuaguuuaagagcuaugcuggaaacagcauagcaaguuuaaauaaggcuaguccguuaucaacuugaaaaaguggcaccgagucggugcuuuuuuu

SEQ ID NO: 6 - RNA, 34 nt, Artificial Sequence
  Synthetic
  misc_feature (1)..(12): n is a, c, g, or u
nnnnnnnnnnnnguuuaagagcuaugcuguuuug

SEQ ID NO: 7 - RNA, 44 nt, Artificial Sequence
  Synthetic
guuaggguuaggguuaggguuaguuuaagagcuaugcuguuuug

SEQ ID NO: 8 - protein, 1631 aa, Artificial Sequence
  Synthetic
GAASPGPKKKRKVDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDEGAPKKKRKVGSSVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 9 - RNA, 33 nt, Artificial Sequence; identical to SEQ ID NO: 3
  Synthetic

SEQ ID NO: 10 - RNA, 86 nt, Artificial Sequence; identical to SEQ ID NO: 2
  Synthetic

(19) United States
(12) Patent Application Publication
(10) Pub. No.: US 2020/0157611 A1
(43) Pub. Date: May 21, 2020

(54) RIBONUCLEOPROTEIN-BASED IMAGING AND DETECTION

(71) Applicant: THE BOARD OF TRUSTEES OF THE LELAND STANFORD JUNIOR UNIVERSITY, Stanford, CA (US)

(72) Inventors: Lei S. Qi, Stanford, CA (US); Haifeng Wang, Stanford, CA (US)

(21) Appl. No.: 16/619,294

(22) PCT Filed: Jun. 4, 2018
(86) PCT No.: PCT/US2018/035834

§ 371 Date: Dec. 4, 2019

Related U.S. Application Data
(60) Provisional application No. 62/515,090, filed on Jun. 5, 2017.

(51) Int. Cl. C12Q 1/6816; C12N 9/22; C12Q 1/6876

(52) U.S. Cl. CPC C12Q 1/6816; C12N 9/22; C12Q 2521/301; C12Q 2563/125; C12Q 1/6876

(57) ABSTRACT

Provided herein is technology relating to biological imaging and diagnostics and particularly, but not exclusively, to methods, systems, kits, and compositions for imaging, detecting, and isolating biological samples using a ribonucleoprotein.

This application claims priority to U.S. provisional patent application Ser. No. 62/515,090, filed Jun. 5, 2017, which is incorporated herein by reference in its entirety.

### STATEMENT REGARDING FEDERALLY SPONSORED RESEARCH OR DEVELOPMENT

This invention was made with Government support under contract EB021240 awarded by the National Institutes of Health. The Government has certain rights in the invention.

### FIELD

Provided herein is technology relating to biological imaging and diagnostics and particularly, but not exclusively, to methods, systems, reagents, kits, and compositions for imaging, detecting, and isolating biological samples using a labeled ribonucleoprotein.

### BACKGROUND

A major challenge for biological imaging and clinical diagnosis is visualizing endogenous genomic loci in living primary cells. Many genetic diseases and pathological processes are associated with chromosome aberrations such as deletions, additions, and translocations. Presently, clinical cytogenetic diagnosis and genomic research rely primarily on fluorescent in situ hybridization (FISH) to visualize sequence-specific genomic loci in cells and tissues. However, FISH requires fixing and permeabilizing biological samples using harsh and lengthy chemical treatments to introduce the fluorescent oligonucleotide probes into cells to allow target binding. Accordingly, medicine and research would benefit from new technologies.

### SUMMARY

Accordingly, provided herein is a technology for robust diagnostic imaging and detection in cells, including living cells such as human primary cells and cells present in an organism in vivo. The technology is based on the introduction of labeled CRISPR/Cas (clustered regularly interspaced short palindromic repeats/CRISPR-associated system) ribonucleoproteins to label, image, detect, and/or isolate nucleic acids with sequence specificity.

Approximately 60% of bacteria and 90% of archaea possess CRISPR/Cas systems (clustered regularly interspaced short palindromic repeats/CRISPR-associated systems) to confer resistance to foreign DNA elements. The type II CRISPR system from Streptococcus pyogenes involves a single gene encoding the Cas9 nuclease protein and a guide RNA duplex comprising a CRISPR RNA (crRNA) and a trans-activating CRISPR RNA (tracr RNA). Targeting of the Cas9-RNA complex to a specific genomic locus is specified by base pairing between RNA (e.g., guided RNA) in the complex and the target site. After binding to the target site, the Cas9 nuclease introduces site-specific double-stranded breaks in DNA. In this way, the CRISPR/Cas system provide microbes with a primitive immune system to silence foreign DNAs.

Recently, variations of the CRISPR/Cas system have been developed for a range of applications, including gene editing and modulating genetic pathways in vivo. For instance, a nuclease-deactivated Cas9 (dCas9) that maintains sequence-specific binding has been produced by introducing a pair of point mutations in Cas9 that inactivate its nuclease activity but that do not affect interaction with the RNA components or the sequence specificity of the complex. Further, producing the Cas9/crRNA/tracrRNA complex has been simplified by fusing the separate crRNA and tracrRNA into a single guide RNA (sgRNA) comprising both components. And, the dCas9 protein may be fused to other proteins or protein domains to direct these proteins to specific genomic locations.

Some CRISPR/Cas9-based technologies have been developed for biological imaging. For instance, dCas9 has been fused to enhanced green fluorescent protein (EGFP) to provide a technology for CRISPR-based live cell genomic imaging (Chen (2013) Cell 155: 1479, incorporated herein by reference). This method was used to visualize coding and noncoding sequences at numerous genomic loci in living human cells (see, e.g., FIG. 3).

Further, many efforts have been made to improve the sensitivity of CRISPR-based genomic imaging and to provide multicolor imaging of multiple genomic loci, e.g., in cultured cell lines. For example, multicolor genomic imaging has been developed using orthogonal dCas9 proteins tagged with different fluorescent proteins or sgRNAs fused to orthogonal protein-interacting RNA aptamers that recruit specific fluorescent proteins. Moreover, efforts have been made to increase the signal-to-noise ratio of CRISPR imaging by recruiting multiple fluorescent proteins to a single genomic locus binding site. For example, methods have been developed in which a dCas9 is fused to a SunTag system protein to recruit up to 24 GFPs per dCas9 (Tanenbaum (2014) Cell 159: 635, incorporated herein by reference). Another method fused an sgRNA to an array of protein-interacting RNA aptamers that bind to multiple RBP-fluorescent proteins. Some imaging systems have fused dCas9 to a modified haloalkane dehalogenase designed to bind covalently to ligands comprising fluorescent groups (Los (2008) ACS Chem. Biol. 3: 373, incorporated herein by reference; see also the PROMEGA HALOTAG system). In addition, strategies are being improved to deliver multiple sgRNAs into a single cell to achieve efficient imaging of non-repetitive sequences.

Nevertheless, current CRISPR-based imaging technologies have fundamental limitations that hinder useful imaging of living cells (e.g., primary cells) for diagnostics and research. For example, present CRISPR-based genomic imaging tools require time-consuming cloning procedures and production of stable cell lines in vitro. Further, the components of current CRISPR imaging systems are delivered into cells as DNA, which imposes difficulties in adjusting expression levels of components to required levels for imaging. As a result, present methods for CRISPR genomic imaging are based on producing and screening stable cell lines, thus rendering the technologies inappropriate for imaging primary cells. Moreover, current multicolor imaging systems and signal amplification systems require delivering multiple large components into cells, which is inefficient and difficult. Finally, most CRISPR imaging based on fluorescent proteins fused to dCas9 or bound to RNA aptamers has an problematic signal-to-noise problems due to unbound proteins that create a diffusive background within the nucleus, thus blurring the on-target signal.

Moreover, understanding changes in local chromosomal structure (e.g., cis-regulatory elements) and regulation of genes during development and disease processes benefits from detecting locus-specific chromosomal interactions. However, this remains challenging due to lack of efficient technologies and research tools. Accordingly, in some embodiments, the technology described herein provides a cloning-free, CRISPR-based technology to detect locus-specific chromatin interaction in living cells, e.g., using affinity-tagged nucletides.

In some embodiments, the technology comprises use of a fluorescent Cas9 to target and/or label RNAs. In some embodiments, the technology relates to use of a labeled guide RNA that forms a complex (e.g., an RNP) with a RNA-directed nuclease to label and visualize RNA transcripts (e.g., an mRNA, a non-coding RNA (e.g., rRNA, microRNA, tRNA, siRNA, snoRNA, exRNA, scaRNA, piRNA, shRNA, Xist, HOTAIR, short non-coding RNA, long non-coding RNA, etc.)) (see, e.g., Nelles et al. (2016) “Programmable RNA Tracking in Live Cells with CRISPR/Cas9” Cell 165: 488, incorporated herein by reference). In some embodiments, the technology comprises use of an RNA-targeting protein (e.g., Cas13, a dCas13), which works according to a similar mechanism as Cas9.

In addition to targeting genomic DNA, Cas9 and other CRISPR related proteins (e.g.

Cas13) also target RNAs directed by gRNAs (see, e.g., Abudayyeh et al. (2017) “RNA targeting with CRISPR-Cas13” Nature 550: 280, incorporated herein by reference). Thus, in some embodiments, labeled gRNAs complex with dCas9 or other RNA-guided nucleases (e.g., a class 2 type VI RNA-guided RNA-targeting CRISPR-Cas effector (e.g., Cas13, dCas13)) to visualize and track dynamics of sequence-specific RNA transcripts and non-coding RNAs in cells. Accordingly, in some embodiments, the technology relates to labeling RNAs using fluorescent guide RNAs in complex with a dCas9 or an RNA-targeting Cas13 or dCas13.

The technology is not limited to any particular RNA-guided nuclease and includes RNA-guided nucleases such as, e.g., Cas9, Cpf1, Cas13, their “d” (e.g., nuclease deficient) versions, and other RNA-guided nucleases known in the art or that function according to the technology described herein.

In some embodiments, provided herein is a rapid, cloning-free, CRISPR-based technology that uses a simple system to label endogenous loci in cells, including living cells (e.g., primary cells). In some embodiments, the technology provides a cytogenetic tool for rapid diagnosis of genetic and chromosomal abnormalities (e.g., Patau syndrome (trisomy 13) and Down syndrome (trisomy 21)) in patient-derived living cells. The technology provides a highly sensitive and flexibility technology for live cell genomic imaging.

As described herein, some embodiments the technology comprise use of a ribonucleoprotein complex that is delivered into a cell. For example, some embodiments of this technology comprise use of a fluorescent guide RNA and a purified dCas9 that is delivered into living cells. As demonstrated by experiments conducted during the development of embodiments of the technology, the technology dramatically increased the sensitivity of genomic imaging when compared to previous technologies, including previous methods based on dCas9-GFP fusions (see, e.g., FIG. 1). Additional experiments conducted during the development of embodiments of the technology indicated successful multi-locus genomic imaging in both cell lines and human primary T lymphocytes using guide RNAs labeled with different dyes. Additional experiments demonstrated the utility of the technology for cytogenetic studies in living cells, e.g., diagnosis of Patau syndrome in a patient sample. In some embodiments, the technology described herein provides compositions related to a cloning-free, CRISPR-based technology to detect locus-specific chromatin interaction in living cells, e.g., using affinity-tagged nucletides.

Accordingly, the technology described herein provides embodiments of methods for imaging a nucleic acid. In some embodiments, methods comprise contacting a nucleic acid with a detectably labeled ribonucleoprotein (RNP) complex comprising a dCas9 and a RNA (e.g., a labeled sgRNA, a labeled crRNA, and/or a labeled tracrRNA); and imaging the nucleic acid by detecting a signal produced by the detectably labeled RNP. In some embodiments, the detectably labeled RNA is a sgRNA; in some embodiments, the detectably labeled RNA is a crRNA and the RNP further comprises a tracrRNA (e.g., a dgRNA system). In some embodiments, the labeled RNA is a tracrRNA. In some embodiments, the detectably labeled RNA is a tracrRNA and the RNP further comprises a crRNA and (e.g., a dgRNA). In some embodiments, the detectably labeled RNA is a tracrRNA and the method comprises use of several crRNAs for several nucleic acid targets to assemble several crRNAs for several targets. In some embodiments, the RNP comprises a detectably labeled dCas9 and one or more RNAs (e.g., a labeled sgRNA, a labeled crRNA, and/or a labeled tracrRNA; and/or, in some embodiments, a sgRNA, a crRNA, and/or a tracrRNA).

In some embodiments, the technology described herein provides methods related to a cloning-free, CRISPR-based technology to detect locus-specific chromatin interaction in living cells, e.g., using affinity-tagged nucletides.

In some embodiments, one or more polypeptides and/or RNAs is produced in vitro. Accordingly, in some embodiments methods further comprise producing the dCas9 in vitro. In some embodiments, methods further comprise producing the detectably labeled RNA in vitro. And, in some embodiments methods further comprise assembling the RNP in vitro from the dCas9 and the detectably labeled RNA. In some embodiments, the RNP finds use to image, label, detect, identify, isolate, etc. a nucleic acid. In some embodiments, the nucleic acid is a chromosome. In some embodiments, the nucleic acid is an RNA, e.g., a messenger RNA. In some embodiments, the nucleic acid is in a cell. Thus, in some embodiments, methods further comprise delivering the RNP into a cell comprising the nucleic acid. In some embodiments, the cell is a living cell; in some embodiments, the cell is a primary cell.

The methods find use, in some embodiments, in detecting and diagnosing an aneuploidy in a patient. Some embodiments of the technology relate to a method of detecting an aneuploidy in a sample. In some embodiments, the method comprise delivering into a cell a detectably labeled ribonucleoprotein (RNP) complex comprising a dCas9 and a RNA comprising a chromosome-specific nucleotide sequence; acquiring an image of the cell; counting the number of labeled foci (e.g., bright spots, high-intensity regions, bright dots, etc.) in the image, wherein a number of labeled foci that is abnormal indicates that the sample is aneuploidy. In some embodiments, the protein of the RNP is detectably labeled and in some embodiments one or more RNA components of the RNP is detectably labeled (e.g., one or more of a sgRNA, tracrRNA, and/or crRNA).

Some embodiments relate to time-lapse imaging, e.g., acquiring a series of images over time, acquiring a moving image (e.g., a “movie”) over a time, e.g., to obtain time information associate with spatial information. Thus, some embodiments provide a method of detecting chromosomal structure, number, arrangement, etc. (e.g., an aneuploidy) in a sample comprising delivering into a cell a detectably labeled ribonucleoprotein (RNP) complex comprising a dCas9 and a RNA comprising a chromosome-specific nucleotide sequence; acquiring a time-lapse image of the cell; comparing the shapes of tracks made by chromosomes in the time-lapse image, wherein a the shape of the track or a track indicating a particular movement or pattern of nucleic acids (e.g., a chromosome) indicates whether the signal is a positive (e.g., due to the RNP binding to the target) or false positive signal (e.g., due to nonspecific aggregation).

Further embodiments relate to a system for imaging a nucleic acid. In some embodiments, the system comprises a detectably labeled RNP comprising a nucleic acid; and a fluorescence detector. In some embodiments, the system further comprises a microscope. In some embodiments, the system further comprises a computer, e.g., running a program (e.g., software) configured to acquire an image, analyze the image to identify labeled foci in the image, count labeled foci, and/or output a result. In some embodiments, the nucleic acid is a detectably labeled nucleic acid, e.g., an RNA, e.g., a sgRNA. In some embodiments, the detectably labeled nucleic acid is, e.g., a crRNA and the RNP further comprises a second RNA, e.g., a tracrRNA. In some embodiments, the detectably labeled RNA is a detectably labeled tracrRNA. In some embodiments, the RNP comprises a dCas9. In some embodiments, the RNP comprises a detectably labeled dCas9 (e.g., a dCas9-GFP fusion). In some embodiments, the system further comprises an input for a sample. In some embodiments, the system further comprises a component for introducing the RNP into a cell. In some embodiments, the technology described herein provides systems related to a cloning-free, CRISPR-based technology to detect locus-specific chromatin interaction in living cells, e.g., using affinity-tagged nucletides.

In some embodiments, the system is an automated system that receives a sample, contacts the sample with an RNP (e.g., introduces the RNP into a cell of the sample), images the sample, analyzes the sample, and outputs a result.

The technology provides embodiments of kits. For example, in some embodiments, kits are provided for imaging a nucleic acid. In some embodiments, the kit comprises a dCas9 and a detectably labeled RNA. In some embodiments, the detectably labeled RNA is a sgRNA; in some embodiments, the detectably labeled RNA is a crRNA and the kit further comprises a tracrRNA. In some embodiments, the detectably labeled RNA is a trRNA and the kit further comprises one or more crRNAs, e.g., for imaging, detecting, isolating, etc. one or more nucleic acids. In some embodiments, the kit comprises a detectably labeled dCas9 (e.g., a dCas9-GFP). In some embodiments, the technology described herein provides kits related to a cloning-free, CRISPR-based technology to detect locus-specific chromatin interaction in living cells, e.g., using affinity-tagged nucletides.

Embodiments of uses are provided by the technology. For example, in some embodiments, the technology relates to use of a detectably labeled RNP complex comprising a dCas9 and a RNA to image a nucleic acid. In some embodiments of uses, the RNA is a detectably labeled RNA, and, e.g., is a sgRNA; in some embodiments of uses, the detectably labeled RNA is a crRNA and the RNP complex further comprises a tracrRNA. In some embodiments of uses, a living cell comprises the nucleic acid, e.g., a living cell is a primary cell. In some embodiments, the detectably labeled RNA is a tracrRNA that finds used in assembling one or more RNPs with one or more crRNAs. In some embodiments of uses, the nucleic acid is a chromosome. In some embodiments of uses, the technology finds use in detecting an aneuploidy. In some embodiments, the technology described herein finds use in a cloning-free, CRISPR-based technology to detect locus-specific chromatin interaction in living cells, e.g., using affinity-tagged nucletides.

In related embodiments, the technology provides compositions. In some embodiments, the technology provides a composition comprising a detectably labeled RNP complex comprising a dCas9 and a RNA. In some embodiments, the RNA is a detectably labeled RNA, e.g., is a detectably labeled sgRNA. In some embodiments, the detectably labeled RNA is a crRNA and the RNP complex further comprises a tracrRNA. In some embodiments, the detectably labeled RNA comprises a fluorescent label. In some embodiments, the detectably labeled RNA comprises a targeting sequence complementary to a chromosomal locus. In some embodiments, the detectably labeled RNA is a tracrRNA. In some embodiments, the detectably labeled RNP comprises a detectably labeled dCas9 (e.g., a dCas9-GFP). In some embodiments, compositions comprise an affinity tagged gRNA. In some embodiments, compositions comprise a gRNA comprising one member of an interacting pair, e.g., for use in isolating the gRNA (e.g., and any associated proteins and/or nucleic acids) using a second member of the interacting pair. In some embodiments, the two members of the interacting pair bind specifically to each other.

Additional embodiments will be apparent to persons skilled in the relevant art based on the teachings contained herein.

### BRIEF DESCRIPTION OF THE DRAWINGS

These and other features, aspects, and advantages of the present technology will become better understood with regard to the following drawings:

FIG. 1 is a drawing of an embodiment of the technology comprising a fluorescently labeled dCas9 (dCas9-GFP) protein, a fluorescently labeled crRNA (Cy3-crRNA), and a tracrRNA (dgRNA), e.g., in a dgRNA system. A ribonucleoprotein comprising the dCas9, crRNA, and tracrRNA is assembled in vitro and then introduced into cells.

FIG. 2 is a drawing showing a multiplex technology for detecting nucleic acids in a cell. A dCas9 protein is assembled with a tracrRNA and a plurality of distinguishably labeled crRNAs (Cy3-crRNA^{chr3} and A488-crRNA^{chr13}). In the drawing, the top sgRNA complex comprises a first crRNA labeled with a first fluorescent label and the bottom sgRNA complex comprises a second crRNA labeled with a second fluorescent label. The first crRNA comprises a first targeting segment comprising a first nucleotide sequence complementary to a first target nucleic acid. The second crRNA comprises a second targeting segment comprising a second nucleotide sequence complementary to a second target nucleic acid. A first ribonucleoprotein comprising the dCas9, first crRNA, and tracrRNA and a second ribonucleoprotein comprising the dCas9, second crRNA, and tracrRNA are assembled in vitro and then introduced into cells, e.g., to provide multiplex imaging of multiple targets in the same cell.

FIG. 3 is a drawing of an embodiment of the technology comprising a fluorescently labeled dCas9 (dCas9-GFP) protein and a sgRNA. A ribonucleoprotein comprising the dCas9 and sgRNA is assembled in vitro and then introduced into cells.

FIG. 4A shows an image of a cell comprising labeled RNPs (center panel). The dots marked 1-4 are chromosomes that are labeled by the binding of an RNP to a target site. The dot marked 5 is a non-specific aggregate producing a false positive signal. The top-left plot shows the tracks of the dots' movements during the time images were acquired. The axes show distances in micrometers. The tracks of the 5 dots are shown enlarged in the panels marked “Dot 1”, “Dot 2”, “Dot 3”, “Dot 4”, and “Dot 5”. The track made by the nucleus is shown in the panel marked “nuclear”. The top-center panel shows the mean square displacement rate for the five dots. The highest mean square displacement is seen for the false-positive signal generated by aggregations (dot 5), which moves randomly in the cell.

FIG. 4B, center panel, shows the same image and dots as is shown in FIG. 4A. The tracks are shown in each panel after subtracting the nuclear movement. Genomic targets exhibit restricted localized movements relative to the directed nuclear movement (dots 1-4). In contrast, a false-positive signal generated by aggregations (dot 5) moved randomly over a larger area.

FIG. 5A is a drawing showing an embodiment of the technology comprising a labeled crRNA (Atto-crRNA), a labeled tracrRNA (Cy3-tracrRNA), and a dCas9 protein. The crRNA, tracrRNA, and protein are assembled in vitro and then introduced into cells. The top drawing shows the technology comprising use of labeled crRNA and tracrRNA. The bottom drawing shows the use of the technology in which multiple crRNAs (e.g., comprising different sequences to target different nucleic acids) are used with a labeled tracrRNA (Cy3-tracrRNA) and dCas9 protein to assemble multiple RNPs targeting multiple targets in cell.

FIG. 5B is a drawing of an embodiment of the technology in which a labeled crRNA (Cy3-crRNA) and a tracrRNA are assembled in vitro and introduced into a cell expressing a dCas9 (e.g., a dCas9-GFP fusion protein).

FIG. 6 shows drawings of embodiments of the technology in which a RNP (e.g., comprising a dCas9-GFP and a sgRNA) finds use in imaging a chromosome, e.g., to characterize chromatin structure and/or the arrangement of histones. In some embodiments, the RNP modifies the arrangement of histones and the changes are imaged and/or detected. The bottom drawing shows the use of labeled crRNA, labeled tracrRNA, or both labeled crRNA and labeled tracrRNA and a dCas9 to assemble RNPs for multiplexed imaging of multiple sites on a nucleic acid, e.g., a chromosome.

FIG. 7 shows a linescan of raw fluorescent intensity (vertical axis, arbitrary fluorescent units) of labeled chromosome loci. The Cy3-crRNA^{Chr3} shows better signal to backgroundratio (top linescan) than the dCas9-GFP channel (bottom linescan).

FIG. 8 shows a pairwise comparison of the signal to background ratio of chromosome 3 loci labeled using RNP complexes comprising dCas9-GFP (grey bars) and Cy3-crRNA^{Chr3}/tracrRNAs (black bares). The signal-to-background ratios (vertical axis, dimensionless ratio) were calculated by dividing the maximum fluorescence intensity of labeled genomic loci by the average fluorescence intensity in the nucleus. 47 loci in 17 cells were analyzed (horizontal axis).

FIG. 9A shows that DNA-encoded dCas9-EGFP imaging is not suitable for diagnostic imaging (Example 1). The bar plots compare labeling efficacy of chromosome 13 loci using transfection of a plasmid encoding dCas9-GFP and sgRNA. Plasmids were transfected into U2OS and Patau Syndrome patient-derived amniotic fluid cells (AG12070). While chromosome 13 loci were labeled by dCas9-GFP in 8% U2OS cells with excessive dCas9-GFP aggregation in the nucleus, <1% cells express dCas9-GFP in AG12070 cells and chromosome 13 signal were rarely observed.

FIG. 9B shows a bar plot comparing the labeling efficacy of chromosome 13 loci in Patau Syndrome patient-derived amniotic fluid cells using the dCas9-GFP plasmid approach (grey bars) and the Atto565-crRNA fRNP approach (black bars). The fRNP method labeled more loci.

FIG. 10A-10B show that the number of genomic loci labeled by Atto565-crRNA fRNP was consistent with the copy number of chromosome 13 in normal and Patau Syndrome (trisomy 13) patient-derived amniotic fluid cells. 60 cells were counted for each cell type. Histograms from normal cells showed that most cells had 2 or 4 copies of chromosome 13 (FIG. 10A). Histograms from trisomy 13 cells showed that most cells had 3 or 6 copies of chromosome 13 (FIG. 10B).

FIG. 11 is a series of fluorescence microscope images showing representative U2OS cells with Cy3-crRNA Ch3 labeling at different time points (1 hour, 4 hours, 24 hours, and 72 hours) after transfection of the CRISPR RNP complex. Brightfield (BF) image is shown for cells at 1 hour before they re-attached to the culture plate. Nuclear staining dye, Hoechst 33342 (blue), was added 4 hours after transfection. The methods using fluorescently modified RNAs shows enhanced signal-to-background labeling of chromosome loci (chromosome 3) in U2OS cells.

FIG. 12 is a series of fluorescence microscope images comparing fluorescent crRNA (Cy3-crRNA) and dCas9-EGFP labeling for endogenous chromosome 3 in U2OS cells using CRISPR RNP delivery. Two time points (0, 4.5 minutes from the start of recording) are shown. Hoechst 33342 labels the cell nucleus. The dotted lines are used for linescan data in shown in FIG. 13.

FIG. 13 shows data from linescans of the S/G2 phase cells shown in FIG. 12 (Merge, dotted line). These data were used to compare signal-to-background (S/B) ratio for each locus in Cy3-crRNA and dCas9-EGFP channels. In each plot, the peaks seen are in the Cy3 (red) channel (for the crRNA) and the dCas9-EGFP (green) channel remains at a lower or flat level.

FIG. 14 is a set of two plots comparing the S/B ratio of labeled chromosome 3 loci using fluorescent crRNA (Cy3-crRNA; left plot, right data series) and dCas9-EGFP (left plot, left data series). In the left bar plot, bars=average value; error bars=standard deviations; p<0.0001 by t-test. The right box plot shows the calculated ratio between the S/B of the Cy3-crRNA and dCas9-EGFP channels at each locus. Average, SDs, 5% and 95% percentiles are shown. 47 loci in 17 cells were analyzed.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

Provided herein is technology relating to biological imaging and diagnostics and particularly, but not exclusively, to methods, systems, kits, and compositions for imaging biological samples using a ribonucleoprotein.

Previous CRISPR-based genomic imaging applications have generally been implemented by expressing a CRISPR-Cas system within a cell from DNA encoding the protein and RNA components delivered on a vector.

Provided herein are technologies relating to use of dCas9/RNA RNP to provide a safe and non-mutagenic off-the-shelf reagent that is modular and quickly adaptable to a wide range of gene expression control and imaging uses. In some embodiments, this dCas9-based RNP delivery platform is combined with a variety of chemically labeled nucleotides (guide RNAs) to provide rapid multiplexed imaging of genomic loci in living cells. In some embodiments, the technology finds use, e.g., in diagnosis of genetic and/or genomic aberrations (e.g., translocations, deletions, and insertions at the nucleotide, gene, genetic locus, chromosome, and genome level; sequence variations at the nucleotide (e.g., single nucleotide polymorphisms), gene, genetic locus, chromosome, and genome level); karyotyping; and in visualizing genomic dynamics, visualizing spatial and temporal variation in gene regulation, and visualizing spatial and temporal dynamics of multiple genomic loci in genomic research.

In some embodiments, the RNP/RNA delivery platform (e.g., the dCas9-based RNP/RNA delivery platform) is combined with a chemically labeled nucleic acid (e.g., a guide RNA) to provide a technology for isolating genomic loci and/or locus-specific chromatin complexes (e.g., comprising chromatin associated proteins and/or nucleic acids) in living cells. In some embodiments, the technology provides an RNP comprising a gRNA comprising one member of an interacting pair; a component comprising a second member of the interacting pair is used to isolate the gRNA and any genomic loci and/or locus-specific chromatin complexes associated with the gRNA.

In this detailed description of the various embodiments, for purposes of explanation, numerous specific details are set forth to provide a thorough understanding of the embodiments disclosed. One skilled in the art will appreciate, however, that these various embodiments may be practiced with or without these specific details. In other instances, structures and devices are shown in block diagram form. Furthermore, one skilled in the art can readily appreciate that the specific sequences in which methods are presented and performed are illustrative and it is contemplated that the sequences can be varied and still remain within the spirit and scope of the various embodiments disclosed herein.

All literature and similar materials cited in this application, including but not limited to, patents, patent applications, articles, books, treatises, and internet web pages are expressly incorporated by reference in their entirety for any purpose. Unless defined otherwise, all technical and scientific terms used herein have the same meaning as is commonly understood by one of ordinary skill in the art to which the various embodiments described herein belongs. When definitions of terms in incorporated references appear to differ from the definitions provided in the present teachings, the definition provided in the present teachings shall control. The section headings used herein are for organizational purposes only and are not to be construed as limiting the described subject matter in any way.

### Definitions

To facilitate an understanding of the present technology, a number of terms and phrases are defined below. Additional definitions are set forth throughout the detailed description.

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

In addition, as used herein, the term “or” is an inclusive “or” operator and is equivalent to the term “and/or” unless the context clearly dictates otherwise. The term “based on” is not exclusive and allows for being based on additional factors not described, unless the context clearly dictates otherwise. In addition, throughout the specification, the meaning of “a”, “an”, and “the” include plural references. The meaning of “in” includes “in” and “on.”

As used herein, a “nucleic acid” or a “nucleic acid sequence” refers to a polymer or oligomer of pyrimidine and/or purine bases, preferably cytosine, thymine, and uracil, and adenine and guanine, respectively (See Albert L. Lehninger, Principles of Biochemistry, at 793-800 (Worth Pub. 1982), incorporated herein by reference). The present technology contemplates any deoxyribonucleotide, ribonucleotide, or peptide nucleic acid component, and any chemical variants thereof, such as methylated, hydroxymethylated, or glycosylated forms of these bases, and the like. The polymers or oligomers may be heterogenous or homogenous in composition, and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. In some embodiments, a nucleic acid or nucleic acid sequence comprises other kinds of nucleic acid structures such as, for instance, a DNA/RNA helix, peptide nucleic acid (PNA), morpholino nucleic acid (see, e.g., Braasch and Corey, Biochemistry, 2002, 41(14), 4503-4510, incorporated herein by reference) and U.S. Pat. No. 5,034,506, incorporated herein by reference), locked nucleic acid (LNA; see Wahlestedt et al., Proc. Natl. Acad. Sci. U.S.A., 2000, 97, 5633-5638, incorporated herein by reference), cyclohexenyl nucleic acids (see Wang, J. Am. Chem. Soc., 2000, 122, 8595-8602, incorporated herein by reference), and/or a ribozyme. Hence, the term “nucleic acid” or “nucleic acid sequence” may also encompass a chain comprising non-natural nucleotides, modified nucleotides, and/or non-nucleotide building blocks that can exhibit the same function as natural nucleotides (e.g., “nucleotide analogs”); further, the term “nucleic acid sequence” as used herein refers to an oligonucleotide, nucleotide or polynucleotide, and fragments or portions thereof, and to DNA or RNA of genomic or synthetic origin, which may be single or double-stranded, and represent the sense or antisense strand.

The technology described herein relates to imaging a nucleic acid. The technology is not limited in the type of nucleic acid that is imaged. Furthermore, the terms “nucleic acid”, “polynucleotide”, “nucleotide sequence”, and “oligonucleotide” are used interchangeably. They refer to a polymeric form of nucleotides of any length, either deoxyribonucleotides or ribonucleotides, or analogs thereof. Polynucleotides may have any three dimensional structure and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. The term also encompasses nucleic-acid-like structures with synthetic backbones, see, e.g., Eckstein, 1991; Baserga et al., 1992; Milligan, 1993; WO 97/03211; WO 96/39154; Mata, 1997; Strauss-Soukup, 1997; and Samstag, 1996, each of which is incorporated herein by reference. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

The term “nucleotide analog” as used herein refers to modified or non-naturally occurring nucleotides including but not limited to analogs that have altered stacking interactions such as 7-deaza purines (i.e., 7-deaza-dATP and 7-deaza-dGTP); base analogs with alternative hydrogen bonding configurations (e.g., such as Iso-C and Iso-G and other non-standard base pairs described in U.S. Pat. No. 6,001,983 to S. Benner, herein incorporated by reference); non-hydrogen bonding analogs (e.g., non-polar, aromatic nucleoside analogs such as 2,4-difluorotoluene, described by B. A. Schweitzer and E. T. Kool, J. Org. Chem., 1994, 59, 7238-7242, B. A. Schweitzer and E. T. Kool, J. Am. Chem. Soc., 1995, 117, 1863-1872; each of which is herein incorporated by reference); “universal” bases such as 5-nitroindole and 3-nitropyrrole; and universal purines and pyrimidines (such as “K” and “P” nucleotides, respectively; P. Kong, et al., Nucleic Acids Res., 1989, 17, 10373-10383, P. Kong et al., Nucleic Acids Res., 1992, 20, 5149-5152, each of which is incorporated herein by reference). Nucleotide analogs include nucleotides having modification on the sugar moiety, such as dideoxy nucleotides and 2′-O-methyl nucleotides. Nucleotide analogs include modified forms of deoxyribonucleotides as well as ribonucleotides.

“Peptide nucleic acid” means a DNA mimic that incorporates a peptide-like polyamide backbone.

As used herein, the term “% sequence identity” refers to the percentage of nucleotides or nucleotide analogs in a nucleic acid sequence that is identical with the corresponding nucleotides in a reference sequence after aligning the two sequences and introducing gaps, if necessary, to achieve the maximum percent identity. Hence, in case a nucleic acid according to the technology is longer than a reference sequence, additional nucleotides in the nucleic acid, that do not align with the reference sequence, are not taken into account for determining sequence identity. Methods and computer programs for alignment are well known in the art, including BLAST, Align 2, and FASTA.

The term “homology” and “homologous” refers to a degree of identity. There may be partial homology or complete homology. A partially homologous sequence is one that is less than 100% identical to another sequence.

The term “sequence variation” as used herein refers to a difference or multiple differences in nucleic acid sequence between two nucleic acids. For example, a wild-type structural gene and a mutant form of this wild-type structural gene may vary in sequence by the presence of one or more single base substitutions or by deletions and/or insertions of one or more nucleotides. These two forms of the structural gene are said to vary in sequence from one another. A second mutant form of the structural gene may exist. This second mutant form is said to vary in sequence from both the wild-type gene and the first mutant form of the gene.

As used herein, the terms “complementary”, “hybridizable”, or “complementarity” are used in reference to polynucleotides (e.g., a sequence of nucleotides such as an oligonucleotide or a target nucleic acid) related by the base-pairing rules. For example, for the sequence “5′-A-G-T-3′” is complementary to the sequence “3′-T-C-A-5′.” Complementarity may be “partial,” in which only some of the nucleic acid bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids. Either term may also be used in reference to individual nucleotides, especially within the context of polynucleotides. For example, a particular nucleotide within an oligonucleotide may be noted for its complementarity, or lack thereof, to a nucleotide within another nucleic acid strand, in contrast or comparison to the complementarity between the rest of the oligonucleotide and the nucleic acid strand.

In some contexts, the term “complementarity” and related terms (e.g., “complementary”, “complement”) refers to the nucleotides of a nucleic acid sequence that can bind to another nucleic acid sequence through hydrogen bonds, e.g., nucleotides that are capable of base pairing, e.g., by Watson-Crick base pairing or other base pairing. Nucleotides that can form base pairs, e.g., nucleotides that are complementary to one another, are the pairs: cytosine and guanine, thymine and adenine, adenine and uracil, and guanine and uracil. The percentage complementarity need not be calculated over the entire length of a nucleic acid sequence. The percentage of complementarity may be limited to a specific region of which the nucleic acid sequences that are base-paired, e.g., starting from a first base-paired nucleotide and ending at a last base-paired nucleotide. The complement of a nucleic acid sequence as used herein refers to an oligonucleotide which, when aligned with the nucleic acid sequence such that the 5′ end of one sequence is paired with the 3′ end of the other, is in “antiparallel association.” Certain bases not commonly found in natural nucleic acids may be included in the nucleic acids of the present invention and include, for example, inosine and 7-deazaguanine Complementarity need not be perfect; stable duplexes may contain mismatched base pairs or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs.

It is understood in the art that the sequence of a polynucleotide need not be 100% complementary to that of its target nucleic acid to be hybridizable or specifically hybridizable. Moreover, a polynucleotide may hybridize over one or more segments such that intervening or adjacent segments are not involved in the hybridization event (e.g., a loop structure or hairpin structure). A polynucleotide can comprise at least 70%, at least 80%, at least 90%, at least 95%, at least 99%, or 100% sequence complementarity to a target region within the target nucleic acid sequence to which they are targeted. For example, a nucleic acid in which 18 of 20 nucleotides of the nucleic acid are complementary to a target region, and would therefore specifically hybridize, would represent 90 percent complementarity. In this example, the remaining non-complementary nucleotides may be clustered or interspersed with complementary nucleotides and need not be contiguous to each other or to complementary nucleotides. Percent complementarity between particular segments of nucleic acid sequences within nucleic acids can be determined routinely using BLAST programs (basic local alignment search tools) and PowerBLAST programs known in the art (Altschul et al., J. Mol. Biol., 1990, 215, 403-410; Zhang and Madden, Genome Res., 1997, 7, 649-656, each of which is incorporated herein by reference) or by using the Gap program (Wisconsin Sequence Analysis Package, Version 8 for Unix, Genetics Computer Group, University Research Park, Madison, Wis.), using default settings, which uses the algorithm of Smith and Waterman (Adv. Appl. Math., 1981, 2, 482-489, incorporated herein by reference).

Thus, in some embodiments, “complementary” refers to a first nucleobase sequence that is at least 60%, 65%, 70%, 75%, 80%, 85%, 90%, 95%, 97%, 98%, or 99% identical to the complement of a second nucleobase sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, or more nucleobases, or that the two sequences hybridize under stringent hybridization conditions. “Fully complementary” means each nucleobase of a first nucleic acid is capable of pairing with each nucleobase at a corresponding position in a second nucleic acid. For example, in certain embodiments, an oligonucleotide wherein each nucleobase has complementarity to a nucleic acid has a nucleobase sequence that is identical to the complement of the nucleic acid over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, or more nucleobases.

“Mismatch” means a nucleobase of a first nucleic acid that is not capable of pairing with a nucleobase at a corresponding position of a second nucleic acid.

As used herein, the term “hybridization” is used in reference to the pairing of complementary nucleic acids. Hybridization and the strength of hybridization (i.e., the strength of the association between the nucleic acids) is influenced by such factors as the degree of complementary between the nucleic acids, stringency of the conditions involved, and the Tm of the formed hybrid. “Hybridization” methods involve the annealing of one nucleic acid to another, complementary nucleic acid, e.g., a nucleic acid having a complementary nucleotide sequence. The ability of two polymers of nucleic acid containing complementary sequences to find each other and “anneal” or “hybridize” through base pairing interaction is a well-recognized phenomenon. The initial observations of the “hybridization” process by Marmur and Lane, Proc. Natl. Acad. Sci. USA 46:453 (1960) and Doty et al., Proc. Natl. Acad. Sci. USA 46:461 (1960), each of which is incorporated herein by reference, have been followed by the refinement of this process into an essential tool of modern biology. For example, hybridization and washing conditions are now well known and exemplified in Sambrook, J., Fritsch, E. F. and Maniatis, T. Molecular Cloning: A Laboratory Manual, Second Edition, Cold Spring Harbor Laboratory Press, Cold Spring Harbor (1989), particularly Chapter 11 and Table 11.1 therein; and Sambrook, J. and Russell, W., Molecular Cloning: A Laboratory Manual, Third Edition, Cold Spring Harbor Laboratory Press, Cold Spring Harbor (2001), each of which is incorporated herein by reference. The conditions of temperature and ionic strength determine the “stringency” of the hybridization.

As used herein, a “double-stranded nucleic acid” may be a portion of a nucleic acid, a region of a longer nucleic acid, or an entire nucleic acid. A “double-stranded nucleic acid” may be, e.g., without limitation, a double-stranded DNA, a double-stranded RNA, a double-stranded DNA/RNA hybrid, etc. A single-stranded nucleic acid having secondary structure (e.g., base-paired secondary structure) and/or higher order structure (e.g., a stem-loop structure) comprises a “double-stranded nucleic acid”. For example, triplex structures are considered to be “double-stranded”. In some embodiments, any base-paired nucleic acid is a “double-stranded nucleic acid”.

As used herein, the term “genomic locus” or “locus” (plural “loci”) is the specific location of a gene or DNA sequence on a chromosome.

The term “gene” refers to a DNA sequence that comprises control and coding sequences necessary for the production of an RNA having a non-coding function (e.g., a ribosomal or transfer RNA), a polypeptide, or a precursor. The RNA or polypeptide can be encoded by a full length coding sequence or by any portion of the coding sequence so long as the desired activity or function is retained. Thus, a “gene” refers to a DNA or RNA, or portion thereof, that encodes a polypeptide or an RNA chain that has functional role to play in an organism. For the purpose of this invention it may be considered that genes include regions that regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, and locus control regions.

The term “wild-type” refers to a gene or a gene product that has the characteristics of that gene or gene product when isolated from a naturally occurring source. A wild-type gene is that which is most frequently observed in a population and is thus arbitrarily designated the “normal” or “wild-type” form of the gene. In contrast, the term “modified,” “mutant,” or “polymorphic” refers to a gene or gene product that displays modifications in sequence and or functional properties (i.e., altered characteristics) when compared to the wild-type gene or gene product. It is noted that naturally-occurring mutants can be isolated; these are identified by the fact that they have altered characteristics when compared to the wild-type gene or gene product.

As used herein the term “variant” should be taken to mean the exhibition of qualities that have a pattern that deviates from what occurs in nature.

The terms “non-naturally occurring” or “engineered” are used interchangeably and indicate the involvement of the hand of man. The terms, when referring to nucleic acid molecules or polypeptides mean that the nucleic acid molecule or the polypeptide is at least substantially free from at least one other component with which they are naturally associated in nature and as found in nature.

The term “oligonucleotide” as used herein is defined as a molecule comprising two or more deoxyribonucleotides or ribonucleotides, preferably at least 5 nucleotides, more preferably at least about 10 to 15 nucleotides and more preferably at least about 15 to 50 nucleotides (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more nucleotides). The exact size will depend on many factors, which in turn depend on the ultimate function or use of the oligonucleotide. The oligonucleotide may be generated in any manner, including chemical synthesis, DNA replication, reverse transcription, PCR, or a combination thereof.

Because mononucleotides are reacted to make oligonucleotides in a manner such that the 5′ phosphate of one mononucleotide pentose ring is attached to the 3′ oxygen of its neighbor in one direction via a phosphodiester linkage, an end of an oligonucleotide is referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. As used herein, a nucleic acid sequence, even if internal to a larger oligonucleotide, also may be said to have 5′ and 3′ ends. A first region along a nucleic acid strand is said to be upstream of another region if the 3′ end of the first region is before the 5′ end of the second region when moving along a strand of nucleic acid in a 5′ to 3′ direction.

When two different, non-overlapping oligonucleotides anneal to different regions of the same linear complementary nucleic acid sequence, and the 3′ end of one oligonucleotide points towards the 5′ end of the other, the former may be called the “upstream” oligonucleotide and the latter the “downstream” oligonucleotide. Similarly, when two overlapping oligonucleotides are hybridized to the same linear complementary nucleic acid sequence, with the first oligonucleotide positioned such that its 5′ end is upstream of the 5′ end of the second oligonucleotide, and the 3′ end of the first oligonucleotide is upstream of the 3′ end of the second oligonucleotide, the first oligonucleotide may be called the “upstream” oligonucleotide and the second oligonucleotide may be called the “downstream” oligonucleotide.

The terms “peptide” and “polypeptide” and “protein” are used interchangeably herein, and refer to a polymeric form of amino acids of any length, which can include coded and non-coded amino acids, chemically or biochemically modified or derivatized amino acids, and polypeptides having modified peptide backbones.

“Binding” as used herein (e.g., with reference to an RNA-binding domain of a polypeptide) refers to a non-covalent interaction between macromolecules (e.g., between a protein and a nucleic acid). While in a state of non-covalent interaction, the macromolecules are said to be “associated” or “interacting” or “binding” (e.g., when a molecule X is said to interact with a molecule Y, it is meant the molecule X binds to molecule Y in a non-covalent manner). Not all components of a binding interaction need be sequence-specific (e.g., contacts with phosphate residues in a DNA backbone), but some portions of a binding interaction may be sequence specific. Binding interactions are generally characterized by a dissociation constant (K_{d}) of less than 10⁻⁶ M, less than 10⁻⁷ M, less than 10⁻⁸ M, less than 10⁻⁹ M, less than 10⁻¹⁰ M, less than 10⁻¹¹ M, less than 10⁻¹² M, less than 10⁻¹³ M, less than 10⁻¹⁴ M, or less than 10⁻¹⁵ M. “Affinity” refers to the strength of binding, increased binding affinity being correlated with a lower K_{d}.

By “binding domain” it is meant a protein domain that is able to bind non-covalently to another molecule. A binding domain can bind to, for example, a DNA molecule (a DNA-binding protein), an RNA molecule (an RNA-binding protein) and/or a protein molecule (a proteinbinding protein). In the case of a protein domain-binding protein, it can bind to itself (to form homodimers, homotrimers, etc.) and/or it can bind to one or more molecules of a different protein or proteins.

As used herein, the term “ribonucleoprotein”, abbreviated “RNP” refers to a multimolecular complex comprising a polypeptide (e.g., a Cas9 or dCas9 protein or a protein having an activity similar to a Cas9 or a dCas9) and a ribonucleic acid (e.g., a sgRNA, a dgRNA). In some embodiments, the polypeptide and ribonucleic acid are bound by a non-covalent interaction.

As used herein, the term “fRNP” refers to a RNP comprising a detectable label. In some embodiments, the detectable label is a fluorescent label. In some embodiments, the polypeptide comprises the detectable label and in some embodiments a RNA comprises the detectable label. In some embodiments, a crRNA, a tracrRNA, or a sgRNA comprises a detectable label.

The term “conservative amino acid substitution” refers to the interchangeability in proteins of amino acid residues having similar side chains. For example, a group of amino acids having aliphatic side chains consists of glycine, alanine, valine, leucine, and isoleucine; a group of amino acids having aliphatic-hydroxyl side chains consists of serine and threonine; a group of amino acids having amide containing side chains consisting of asparagine and glutamine; a group of amino acids having aromatic side chains consists of phenylalanine, tyrosine, and tryptophan; a group of amino acids having basic side chains consists of lysine, arginine, and histidine; a group of amino acids having acidic side chains consists of glutamate and aspartate; and a group of amino acids having sulfur containing side chains consists of cysteine and methionine. Exemplary conservative amino acid substitution groups are: valine-leucine/isoleucine, phenylalanine-tyrosine, lysine-arginine, alanine-valine, and asparagine-glutamine

“Recombinant,” as used herein, means that a particular nucleic acid (DNA or RNA) is the product of various combinations of cloning, restriction, polymerase chain reaction (PCR) and/or ligation steps resulting in a construct having a structural coding or non-coding sequence distinguishable from endogenous nucleic acids found in natural systems. DNA sequences encoding polypeptides can be assembled from cDNA fragments or from a series of synthetic oligonucleotides, to provide a synthetic nucleic acid which is capable of being expressed from a recombinant transcriptional unit contained in a cell or in a cell-free transcription and translation system. Genomic DNA comprising the relevant sequences can also be used in the formation of a recombinant gene or transcriptional unit. Sequences of non-translated DNA may be present 5′ or 3′ from the open reading frame, where such sequences do not interfere with manipulation or expression of the coding regions, and may indeed act to modulate production of a desired product by various mechanisms). Alternatively, DNA sequences encoding RNA (e.g., DNA-targeting RNA) that is not translated may also be considered recombinant. Thus, e.g., the term “recombinant” nucleic acid refers to one which is not naturally occurring, e.g., is made by the artificial combination of two otherwise separated segments of sequence through human intervention. This artificial combination is often accomplished by either chemical synthesis means, or by the artificial manipulation of isolated segments of nucleic acids, e.g., by genetic engineering techniques. Such is usually done to replace a codon with a codon encoding the same amino acid, a conservative amino acid, or a non-conservative amino acid. Alternatively, it is performed to join together nucleic acid segments of desired functions to generate a desired combination of functions. This artificial combination is often accomplished by either chemical synthesis means, or by the artificial manipulation of isolated segments of nucleic acids, e.g., by genetic engineering techniques. When a recombinant polynucleotide encodes a polypeptide, the sequence of the encoded polypeptide can be naturally occurring (“wild type”) or can be a variant (e.g., a mutant) of the naturally occurring sequence. Thus, the term “recombinant” polypeptide does not necessarily refer to a polypeptide whose sequence does not naturally occur. Instead, a “recombinant” polypeptide is encoded by a recombinant DNA sequence, but the sequence of the polypeptide can be naturally occurring (“wild type”) or non-naturally occurring (e.g., a variant, a mutant, etc.). Thus, a “recombinant” polypeptide is the result of human intervention, but may be a naturally occurring amino acid sequence.

A “vector” or “expression vector” is a replicon, such as plasmid, phage, virus, or cosmid, to which another DNA segment, i.e. an “insert”, may be attached so as to bring about the replication of the attached segment in a cell.

A cell has been “genetically modified” or “transformed” or “transfected” by exogenous DNA, e.g. a recombinant expression vector, when such DNA has been introduced inside the cell. The presence of the exogenous DNA results in permanent or transient genetic change. The transforming DNA may or may not be integrated (covalently linked) into the genome of the cell. In prokaryotes, yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones that comprise a population of daughter cells containing the transforming DNA. A “clone” is a population of cells derived from a single cell or common ancestor by mitosis. A “cell line” is a clone of a primary cell that is capable of stable growth in vitro for many generations.

Suitable methods of genetic modification (also referred to as “transformation”) include e.g., viral or bacteriophage infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery (see, e.g., Panyam and Labhasetwar (2012), Advanced Drug Delivery Reviews, 64 (supplement); 61-71, incorporated herein by reference). The choice of method of genetic modification is generally dependent on the type of cell being transformed and the circumstances under which the transformation is taking place (e.g., in vitro, ex vivo, or in vivo). A general discussion of these methods can be found in Ausubel, et al., Short Protocols in Molecular Biology, 3rd ed., Wiley & Sons, 1995, incorporated herein by reference.

A “target nucleic acid” (e.g., a “target DNA”) as used herein is a polynucleotide (nucleic acid, gene, chromosome, genome, etc.) that comprises a “target site” or “target sequence.” The terms “target site” or “target sequence” are used interchangeably herein to refer to a nucleic acid sequence present in a target DNA to which a DNA-targeting segment of a DNA-targeting RNA will bind, provided sufficient conditions for binding exist. Suitable DNA/RNA binding conditions include physiological conditions normally present in a cell. Other suitable DNA/RNA binding conditions (e.g., conditions in a cell-free system) are known in the art; see, e.g., Sambrook, referenced herein and incorporated by reference. The strand of the target DNA that is complementary to and hybridizes with the DNA-targeting RNA is referred to as the “complementary strand” and the strand of the target DNA that is complementary to the “complementary strand” (and is therefore not complementary to the DNA-targeting RNA) is referred to as the “noncomplementary strand” or “non-complementary strand”.

The RNA molecule that binds to the polypeptide in the RNP and targets the polypeptide to a specific location within the target DNA is referred to herein as the “DNA targeting RNA” or “DNA-targeting RNA polynucleotide” (also referred to herein as a “guide RNA” or “gRNA”). A DNA-targeting RNA comprises two segments, a “DNA-targeting segment” and a “protein-binding segment.” In some embodiments, the gRNA comprises two RNAs (e.g., a dgRNA, e.g., a crRNA and a tracrRNA) and in some embodiments the gRNA comprises one RNA (e.g., a sgRNA).

By “segment” it is meant a segment or section or portion or region of a molecule, e.g., a contiguous segment of nucleotides in an RNA, DNA, or protein. A segment can also mean a segment or section or portion or region of a complex such that a segment may comprise regions of more than one molecule. For example, in some cases the protein-binding segment (described below) of a DNA targeting RNA is one RNA molecule and the protein-binding segment therefore comprises a region of that RNA molecule. In other cases, the protein-binding segment (described below) of a DNA-targeting RNA comprises two separate molecules that are hybridized along a region of complementarity. As an illustrative, non-limiting example, a protein-binding segment of a DNA targeting RNA that comprises two separate molecules can comprise (i) base pairs 40-75 of a first RNA molecule that is 100 base pairs in length; and (ii) base pairs 10-25 of a second RNA molecule that is 50 base pairs in length. The definition of “segment,” unless otherwise specifically defined in a particular context, is not limited to a specific number of total base pairs, is not limited to any particular number of base pairs from a given RNA molecule, is not limited to a particular number of separate molecules within a complex, and may include regions of RNA molecules that are of any total length and may or may not include regions with complementarity to other molecules.

The DNA-targeting segment (or “DNA-targeting sequence”) comprises a nucleotide sequence that is complementary to a specific sequence within a target DNA (the complementary strand of the target DNA). The protein-binding segment (or “protein-binding sequence”) interacts with a polypeptide of the RNP. The protein-binding segment of a DNA-targeting RNA comprises two complementary segments of nucleotides that hybridize to one another to form a double stranded RNA duplex (dsRNA duplex).

A DNA-targeting RNA and a polypeptide form a RNP complex (e.g., bind via non-covalent interactions). The DNA-targeting RNA provides target specificity to the RNP complex by comprising a nucleotide sequence that is complementary to a sequence of a target DNA. The polypeptide of the RNP complex provides site-specific binding and, in some embodiments, labeling (e.g., for imaging). In other words, the polypeptide of the RNP is guided to a target DNA sequence (e.g. a target sequence in a chromosomal nucleic acid; a target sequence in an extrachromosomal nucleic acid, e.g. an episomal nucleic acid, a minicircle, etc.; a target sequence in a mitochondrial nucleic acid; a target sequence in a chloroplast nucleic acid; a target sequence in a plasmid; etc.) by virtue of its association with the protein-binding segment of the DNA-targeting RNA.

In some embodiments, a DNA-targeting RNA comprises two separate RNA molecules (e.g., two RNA polynucleotides, e.g., an “activator-RNA” and a “targeter-RNA”) and is referred to herein as a “double-molecule DNA-targeting RNA” or a “two-molecule DNA-targeting RNA” or a “double guide RNA” or a “dgRNA”. In other embodiments, the DNA-targeting RNA is a single RNA molecule (e.g., a single RNA polynucleotide) and is referred to herein as a “single-molecule DNA-targeting RNA,” a “single guide RNA,” or an “sgRNA.” The term “DNA-targeting RNA” or “guide RNA” or “gRNA” is inclusive, referring both to double-molecule DNA-targeting RNAs (dgRNAs) and to single-molecule DNA-targeting RNAs (sgRNAs).

An exemplary two-molecule DNA-targeting RNA comprises a crRNA-like (“CRISPR RNA” or “targeter-RNA” or “crRNA” or “crRNA repeat”) molecule and a corresponding tracrRNA-like (“trans-acting CRISPR RNA” or “activator-RNA” or “tracrRNA”) molecule. A crRNA-like molecule (targeter-RNA) comprises both the DNA-targeting segment (single stranded) of the DNA-targeting RNA and a region (“duplex-forming segment”) that forms one half of the dsRNA duplex of the protein-binding segment of the DNA-targeting RNA. A corresponding tracrRNA-like molecule (activator-RNA) comprises a region (duplex-forming segment) that forms the other half of the dsRNA duplex of the protein-binding segment of the DNA-targeting RNA. In other words, a portion of the crRNA-like molecule is complementary to and hybridizes with a portion of a tracrRNA-like molecule to form the dsRNA duplex of the protein-binding domain of the DNA-targeting RNA. As such, each crRNA-like molecule can be said to have a corresponding tracrRNA-like molecule. The crRNA-like molecule additionally provides the single stranded DNA-targeting segment.

Thus, a crRNA-like molecule (e.g., a crRNA) and a tracrRNA-like molecule (e.g., a tracrRNA) hybridize (as a corresponding pair) to form a DNA-targeting RNA. The exact sequence of a given crRNA or tracrRNA molecule is characteristic of the species in which the RNA molecules are found. Various crRNAs and tracrRNAs are known in the art. A subject double molecule DNA-targeting RNA (dgRNA) can comprise any corresponding crRNA and tracrRNA pair. A subject double-molecule DNA-targeting RNA (sgRNA) can comprise any corresponding crRNA and tracrRNA pair.

The term “activator-RNA” is used herein to mean a tracrRNA-like molecule of a double molecule DNA-targeting RNA (e.g., a tracrRNA). The term “targeter-RNA” is used herein to mean a crRNA-like molecule of a double-molecule DNA-targeting RNA (e.g., a crRNA). The term “duplex-forming segment” is used herein to mean the segment of an activator-RNA or a targeter-RNA that contributes to the formation of the dsRNA duplex by hybridizing to a segment of a corresponding activator-RNA or targeter-RNA molecule. In other words, an activator-RNA comprises a duplex-forming segment that is complementary to the duplex-forming segment of the corresponding targeter-RNA. As such, an activator-RNA comprises a duplex-forming segment while a targeter-RNA comprises both a duplex-forming segment and the DNA-targeting segment of the DNA-targeting RNA. Therefore, a subject double-molecule DNA-targeting RNA can be comprised of any corresponding activator-RNA and targeter-RNA pair.

As used herein, “CRISPR system” refers collectively to transcripts and other elements involved in the expression of and/or directing the activity of CRISPR-associated (“Cas”) genes, including sequences encoding a Cas gene or dCas gene, a tracr (trans-activating CRISPR) sequence (e.g., tracrRNA or an active partial tracrRNA), a cr (CRISPR) sequence (e.g., crRNA or an active partial crRNA), or other sequences and transcripts from a CRISPR locus. In embodiments of the invention, the terms guide sequence and guide RNA (gRNA) are used interchangeably. In some embodiments, one or more elements of a CRISPR system is derived from a type I, type II, or type III CRISPR system. In some embodiments, one or more elements of a CRISPR system is derived from a particular organism comprising an endogenous CRISPR system, such as Streptococcus pyogenes. In general, a CRISPR system is characterized by elements that promote the formation of a CRISPR RNP complex (e.g., in vitro or in vivo) and direct it to the site of a target sequence in a cell (e.g., after introduction of the RNP).

As used herein, the terms “subject” and “patient” refer to any organisms including plants, microorganisms, and animals (e.g., mammals such as dogs, cats, livestock, and humans).

The terms “treatment”, “treating”, and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease or symptom in a mammal, and includes: (a) preventing the disease or symptom from occurring in a subject which may be predisposed to acquiring the disease or symptom but has not yet been diagnosed as having it; (b) inhibiting the disease or symptom, e.g., arresting its development; or (c) relieving the disease, e.g., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy will desirably be administered during the symptomatic stage of the disease, and in some cases after the symptomatic stage of the disease

The term “sample” in the present specification and claims is used in its broadest sense. On the one hand it is meant to include a specimen or culture (e.g., microbiological cultures). On the other hand, it is meant to include both biological and environmental samples. A sample may include a specimen of synthetic origin.

As used herein, a “biological sample” refers to a sample of biological tissue or fluid. For instance, a biological sample may be a sample obtained from an animal (including a human); a fluid, solid, or tissue sample; as well as liquid and solid food and feed products and ingredients such as dairy items, vegetables, meat and meat by-products, and waste. Biological samples may be obtained from all of the various families of domestic animals, as well as feral or wild animals, including, but not limited to, such animals as ungulates, bear, fish, lagomorphs, rodents, etc. Examples of biological samples include sections of tissues, blood, blood fractions, plasma, serum, urine, or samples from other peripheral sources or cell cultures, cell colonies, single cells, or a collection of single cells. Furthermore, a biological sample includes pools or mixtures of the above mentioned samples. A biological sample may be provided by removing a sample of cells from a subject, but can also be provided by using a previously isolated sample. For example, a tissue sample can be removed from a subject suspected of having a disease by conventional biopsy techniques. In some embodiments, a blood sample is taken from a subject. A biological sample from a patient means a sample from a subject suspected to be affected by a disease.

Environmental samples include environmental material such as surface matter, soil, water, and industrial samples, as well as samples obtained from food and dairy processing instruments, apparatus, equipment, utensils, disposable and non-disposable items. These examples are not to be construed as limiting the sample types applicable to the present invention.

The term “label” as used herein refers to any atom or molecule that can be used to provide a detectable (preferably quantifiable) effect, and that can be attached to a nucleic acid or protein. Labels include, but are not limited to, dyes (e.g., fluorescent dyes or moieties); radiolabels such as ³²P; binding moieties such as biotin; haptens such as digoxgenin; luminogenic, phosphorescent, or fluorogenic moieties; mass tags; and fluorescent dyes alone or in combination with moieties that can suppress or shift emission spectra by fluorescence resonance energy transfer (FRET). Labels may provide signals detectable by fluorescence, radioactivity, colorimetry, gravimetry, X-ray diffraction or absorption, magnetism, enzymatic activity, characteristics of mass or behavior affected by mass (e.g., MALDI time-of-flight mass spectrometry; fluorescence polarization), and the like. A label may be a charged moiety (positive or negative charge) or, alternatively, may be charge neutral. Labels can include or consist of nucleic acid or protein sequence, so long as the sequence comprising the label is detectable. In some embodiments, a label is a “contrast agent” used, e.g., for computerized tomography (CT), magnetic resonance imaging (MRI), ultrasound, X-ray based techniques, ultrasound, optical imaging modalities, Overhauser MR (OMRI), oxygen imaging (OXI), magnetic source imaging (MSI), applied potential tomography (APT), and imaging methods based on microwaves. Non-limiting examples of contrast agents include, e.g., radiocontrast agents (e.g., iodine, barium); gadolinium; 99m-technetium; magnetic materials; thallium; F-18 labeled molecules (e.g., ¹⁸F-labelled glucose (┌¹⁸F┐FDG)); and metal-chelate complexes.

As used herein, “moiety” refers to one of two or more parts into which something may be divided, such as, for example, the various parts of an oligonucleotide, a molecule, a chemical group, a domain, a probe, etc.

As used herein, a “stem-loop structure” refers to a nucleic acid having a secondary structure that includes a region of nucleotides that are known or predicted to form a double strand (stem portion) that is linked on one side to a region of predominantly single-stranded nucleotides (loop portion). The terms “hairpin” and “fold-back” structures are also used herein to refer to stem-loop structures. Such structures are well known in the art and these terms are used consistently with their known meanings in the art. As is known in the art, a stem-loop structure does not require exact basepairing. Thus, the stem may include one or more base mismatches. Alternatively, the basepairing may be exact, e.g., not include any mismatches

As used herein, the term “directly measuring” or “directly imaging” refers to the direct magnification, visualization, imaging, and/or measuring of a signal (e.g., produced by a detectable label) using a detection system such as a microscope. That is, the signal is directly observed using the imaging system; in some cases, the actual quantitative value is determined. For example, the imaging systems described herein permit direct measure of the location (in two dimensions (e.g., in the XY focal plane), in three dimensions (comprising both XY focal plane and depth with respect to the focal plane), time, intensity, change in intensity with time, etc.

### Description

Analysis and evaluation of genetic status would benefit from a rapid, inexpensive, and accurate technology for imaging nucleic acids (e.g., chromosomes) in living primary cells. Such a technology finds use in, e.g., point-of-care diagnosis of prenatal disorders and cancers. Moreover, dynamic genomic tracking in primary cells improves understanding in art relating to the landscape of temporal-spatial nuclear organization during disease-relevant processes. Accordingly, embodiments of a technology are described herein that provide adaptable chromosome tracking and diagnosis in primary cells. In particular, embodiments of the technology relate to a versatile CRISPR fluorescent ribonucleoprotein (fRNP) approach for rapid and efficient dynamic monitoring of multiple genomic loci. The technology provides a diagnostic imaging technology for use in living cells, including cell lines and hard-to-transfect cells such as primary human T lymphocytes. The technology provides a rapid and robust detection of chromosomal aberrations, e.g., Patau Syndrome (trisomy 13), Down Syndrome (trisomy 21), etc., in living cells, e.g., in prenatal amniotic fluid cells. Experiments conducted during the development of embodiments of the technology produced data indicating that real-time monitoring and analysis of chromatin dynamics in patient cells improves the identification of true positive targets, potentially reducing false positive detection. Thus, the technology provides a rapid, dynamic, cost-effective genomic visualization method that finds use as a new tool for biological research and cytogenetic diagnosis in living primary cells.

Moreover, understanding changes in local chromosomal structure (e.g., cis-regulatory elements) and regulation of genes during development and disease processes benefits from detecting locus-specific chromosomal interactions. However, this remains challenging due to lack of efficient technologies and research tools. Accordingly, in some embodiments, the technology described herein provides a cloning-free, CRISPR-based technology to detect locus-specific chromatin interaction in living cells, e.g., using affinity-tagged nucletides.

### RNP Complexes, Polypeptides, Ribonucleic Acids

The technology comprises use of a ribonucleoprotein (RNP) complex comprising a Cas9 or Cas9-like protein and an RNA (e.g., e.g., a gRNA, a subject DNA-targeting RNA, an activator-RNA and a targeter-RNA, a crRNA and a tracrRNA; a dgRNA; a sgRNA). In some embodiments, the Cas9 is an enzymatically inactive, or “dead”, Cas9 protein (“dCas9”).

The RNA provides target specificity to the RNP complex by comprising a nucleotide sequence that is complementary to a sequence of a target DNA. The polypeptide of the complex provides binding activity. In other words, the polypeptide is guided to a DNA sequence (e.g. a chromosomal sequence or an extrachromosomal sequence, e.g. an episomal sequence, a minicircle sequence, a mitochondrial sequence, a chloroplast sequence, etc.) by virtue of its association with at least the protein-binding segment of the DNA-targeting RNA.

While various CRISPR/Cas systems have been used extensively for genome editing in cells of various types and species, recombinant and engineered nucleic acid-binding proteins such as Cas9 and Cas9-like proteins find use in the present technology to direct detectable labels to specific nucleic acids for imaging. Embodiments of the technology provide an RNP comprising a polypeptide, e.g., a Cas9, dCas9, or related or similar protein. The Cas9 protein was discovered as a component of the bacterial adaptive immune system (see, e.g., Barrangou et al. (2007) “CRISPR provides acquired resistance against viruses in prokaryotes” Science 315: 1709-1712, incorporated herein by reference). Cas9 is an RNA-guided endonuclease that targets and destroys foreign DNA in bacteria using RNA:DNA base-pairing between a guide RNA (gRNA) and foreign DNA to provide sequence specificity. Recently, Cas9/gRNA complexes (e.g., a Cas9/gRNA RNP) have found use in genome editing (see, e.g., Doudna et al. (2014) “The new frontier of genome engineering with CRISPR-Cas9” Science 346: 6213, incorporated herein by reference).

Accordingly, some Cas9/RNA RNP complexes comprise two RNA molecules: (1) a CRISPR RNA (crRNA), possessing a nucleotide sequence complementary to the target nucleotide sequence; and (2) a trans-activating crRNA (tracrRNA). In this mode, Cas9 functions as an RNA-guided nuclease that uses both the crRNA and tracrRNA to recognize and cleave a target sequence. Recently, a single chimeric guide RNA (sgRNA) mimicking the structure of the annealed crRNA/tracrRNA has become more widely used than crRNA/tracrRNA because the gRNA approach provides a simplified system with only two components (e.g., the Cas9 and the sgRNA). Thus, sequence-specific binding of the RNP to a nucleic acid can be guided by a dual-RNA complex (e.g., a “dgRNA”), e.g., comprising a crRNA and a tracrRNA in two separate RNAs or by a chimeric single-guide RNA (e.g., a “sgRNA”) comprising a crRNA and a tracrRNA in a single RNA. (see, e.g., Jinek et al. (2012) “A Programmable Dual-RNA-Guided DNA Endonuclease in Adaptive Bacterial Immunity” Science 337:816-821, incorporated herein by reference).

As used herein, the targeting region of a crRNA (2-RNA dgRNA system) or a sgRNA (single guide system) is referred to as the “guide RNA” (gRNA). In some embodiments, the gRNA comprises, consists of, or essentially consists of 10 to 50 bases, e.g., 15 to 40 bases, e.g., 15 to 30 bases, e.g., 15 to 25 bases (e.g., 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 bases). Methods are known in the art for determining the length of the gRNA that provides the most efficient target recognition for a Cas9. See, e.g., Lee et al. (2016) “The Neisseria meningitidis CRISPR-Cas9 System Enables Specific Genome Editing in Mammalian Cells” Molecular Therapy 24: 645 (2016), incorporated herein by reference.

Accordingly, in some embodiments the gRNA is a short synthetic RNA comprising a “scaffold sequence” (protein-binding segment) for Cas9 binding and a user-defined “DNA-targeting sequence” (DNA-targeting segment) that is approximately 20-nucleotides long and is complementary to the target site of the target nucleic acid.

In some embodiments, DNA targeting specificity is determined by two factors: 1) a DNA sequence matching the gRNA targeting sequence and a protospacer adjacent motif (PAM) directly downstream of the target sequence. Some Cas9/gRNA complexes recognize a DNA sequence comprising a protospacer adjacent motif (PAM) sequence and an adjacent sequence comprising approximately 20 bases complementary to the gRNA. Canonical PAM sequences are NGG or NAG for Cas9 from Streptococcus pyogenes and NNNNGATT for the Cas9 from Neisseria meningitidis. Following DNA recognition by hybridization of the gRNA to the DNA target sequence, Cas9 cleaves the DNA sequence via an intrinsic nuclease activity. For genome editing and other purposes, the CRISPR/Cas system from S. pyogenes has been used most often. Using this system, one can target a given target nucleic acid (e.g., for editing or other manipulation) by designing a gRNA compressing a nucleotide sequence complementary to a DNA sequence (e.g., a DNA sequence comprising approximately 20 nucleotides) that is 5′-adjacent to the PAM. Methods are known in the art for determining a PAM sequence that provides efficient target recognition for a Cas9. See, e.g., Zhang et al. (2013) “Processing-independent CRISPR RNAs limit natural transformation in Neisseria meningitidis” Molecular Cell 50: 488-503, incorporated herein by reference; Lee et al., supra, incorporated herein by reference.

In some exemplary embodiments, the crRNA comprise a sequence according to SEQ ID NO: 6

NNNNNNNNNNNNrGrUrUrUrArArGrArGrCrUrArUrGrCrUrGrUr
UrUrUrG

where the “NNNNNNNNNNNN” represents the DNA-targeting sequence that is complementary to the target sequence (e.g., of a nucleic acid to be imaged). In some embodiments, the 5′ end of the crRNA comprises a detectable label, e.g., a dye, e.g., a fluorescent dye.

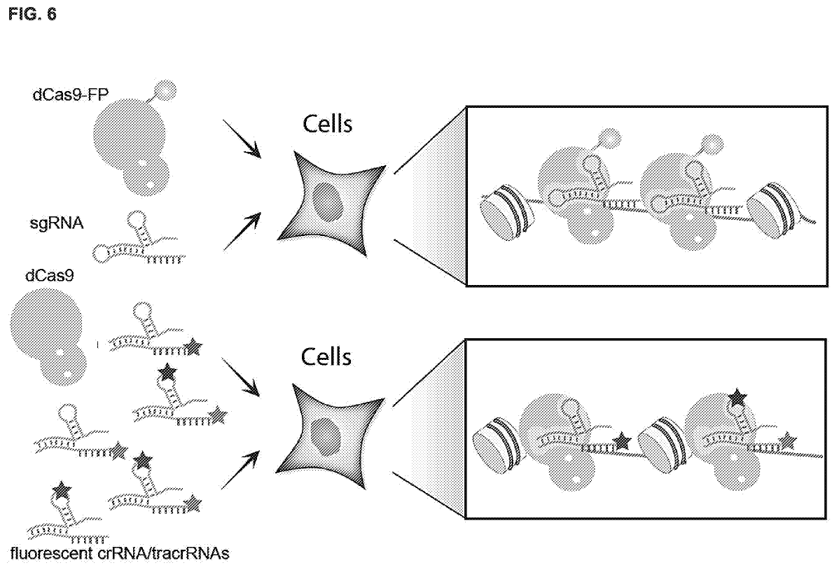

In some embodiments, the tracrRNA comprises a sequence of a naturally occurring tracrRNA, e.g., a provided by FIGS. 6, 35, and 37, and by SEQ ID NOs: 267-272 and 431-562 of U.S. Pat. App. Pub. No. 20170051312, incorporated herein by reference.

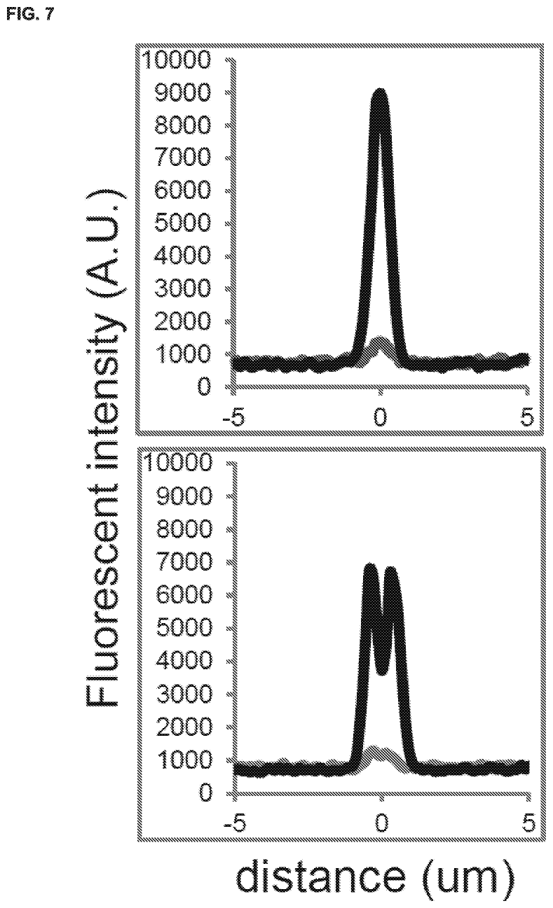

In some embodiments, the crRNA comprises a sequence that hybridizes to a tracrRNA to form a duplex structure, e.g., a sequence provided by FIG. 7 and SEQ ID NOs: 563-679 of U.S. Pat. App. Pub. No. 20170051312, incorporated herein by reference. In some embodiments, a crRNA comprises a sequence provided by FIG. 37 of U.S. Pat. App. Pub. No. 20170051312, incorporated herein by reference. In some embodiments, the duplex-forming segment of the crRNA is at least about 60% identical to one of the tracrRNA molecules set forth in SEQ ID NOs: 431-679 of U.S. Pat. App. Pub. No. 20170051312, incorporated herein by reference, or a complement thereof.

Thus, in some embodiments, exemplary (but not limiting) nucleotide sequences that are included in a dgRNA system include either of the sequences set forth in U.S. Pat. App. Pub. No. 20170051312, incorporated herein by reference, as SEQ ID NOs: 431-562, or complements thereof pairing with any sequences set forth in U.S. Pat. App. Pub. No. 20170051312, incorporated herein by reference, SEQ ID NOs: 563-679, or complements thereof that can hybridize to form a protein binding segment.

In some embodiments, a single-molecule gRNA (e.g., a sgRNA) comprises two complementary stretches of nucleotides that hybridize to form a dsRNA duplex. In some embodiments, the sgRNA (or a DNA encoding the sgRNA) is at least about 60% identical to one of the tracrRNA molecules set forth in U.S. Pat. App. Pub. No. 20170051312, incorporated herein by reference, as SEQ ID NOs: 431-562, or a complement thereof, over at least 8 contiguous nucleotides. In some embodiments, the sgRNA (or a DNA encoding the sgRNA) is at least about 60% identical to one of the tracrRNA molecules set forth in U.S. Pat. App. Pub. No. 20170051312, incorporated herein by reference, as SEQ ID NOs: 563-679, or a complement thereof, over at least 8 contiguous nucleotides. Appropriate naturally occurring pairs of crRNAs and tracrRNAs can be routinely determined by taking into account the species name and base-pairing (for the dsRNA duplex of the protein-binding domain) when determining appropriate cognate pairs.

In some embodiments, the present technology comprises use of a catalytically inactive form of Cas9 (“dead Cas9” or “dCas9”), in which point mutations are introduced into the nucleotide sequence that encodes the protein to produce amino acid substitutions that minimize, decrease, eliminate, or disable the nuclease activity. In some embodiments, the dCas9 protein is produced from a S. pyogenes Cas9. In some embodiments, the dCas9 protein comprises mutations at, e.g., D10, E762, H983, and/or D986; and at H840 and/or N863, e.g., at D10 and H840, e.g., D10A or D10N and H840A or H840N or H840Y. In some embodiments, the dCas9 is provided as a fusion protein comprising a domain that is detectable.

The dCas9/gRNA complex binds to a target nucleic acid with a sequence specificity provided by the gRNA, but does not cleave the nucleic acid. In this form, the dCas9/gRNA RNP binds to the target nucleic acid with sequence specificity; in some embodiments, the RNP “melts” the target sequence to provide single-stranded regions of the target nucleic acid in a sequence-specific manner (see, e.g., Qi et al. (2013) “Repurposing CRISPR as an RNA-guided platform for sequence-specific control of gene expression” Cell 152(5): 1173-83, incorporated herein by reference).

Furthermore, while the Cas9/gRNA system and dCas9/gRNA system initially targeted sequences adjacent to a PAM, the dCas9/gRNA system as used herein has been engineered to target any nucleotide sequence for binding (e.g., the technologies described herein are PAM-independent). Also, Cas9 and dCas9 orthologs encoded by compact genes (e.g., Cas9 from Staphylococcus aureus) are known (see, e.g., Ran et al. (2015) “In vivo genome editing using Staphylococcus aureus Cas9” Nature 520: 186-191, incorporated herein by reference), which improves the cloning and manipulation of the Cas9 components in vitro.

In some embodiments, different Cas9 proteins (e.g., Cas9 proteins from various species and modified versions (e.g., nuclease-deficient versions) thereof) may be advantageous to use in the various provided methods in order to capitalize on various characteristics of the different Cas9 proteins (e.g., for different PAM sequence preferences; for no PAM sequence requirement; for increased or decreased binding activity; for an increased or decreased level of cellular toxicity; for increase or decrease efficiency of in vitro RNP formation; for increase or decrease ability for introduction into cells (e.g., living cells, e.g., living primary cells), etc.). Cas9 proteins from various species may require different PAM sequences in the target DNA. Thus, for a particular Cas9 protein of choice, the PAM sequence requirement may be different than the 5′-XGG-3′ sequence described above.

In some embodiments, the technology comprises use of other RNA-guide nucleases (e.g., Cpf1 and modified versions (e.g., nuclease-deficient “d” versions) thereof). For example, in some embodiments use of other RNA-guide nucleases (e.g., Cpf1 and modified versions (e.g., nuclease-deficient versions) thereof provides advantages—e.g., in some embodiments the characteristics of the different nucleases are appropriate for methods as described herein (e.g., other RNA-guide nucleases have preferences for different PAM sequence preferences; other RNA-guide nucleases operate using single crRNAs other than cr/tracrRNA complexes; other RNA-guide nucleases operate with shorter guide RNAs, etc.) In some embodiments, the technology comprises use of a Cpf1 enzyme, e.g., as described in U.S. Pat. No. 9,790,490, which is incorporated herein by reference in its entirety.

Many Cas9 orthologs from a wide variety of species have been identified herein and the proteins share only a few identical amino acids. All identified Cas9 orthologs have the same domain architecture with a central HNH endonuclease domain and a split RuvC/RNaseH domain. Cas9 proteins share 4 key motifs with a conserved architecture. Motifs 1, 2, and 4 are RuvC like motifs while motif 3 is an HNH-motif. In some embodiments, a suitable polypeptide (e.g., a Cas9 or dCas9) comprises an amino acid sequence having 4 motifs, each of motifs 1-4 having at least about 75%, at least about 80%, at least about 85%, at least about 90%, at least about 95%, at least about 99% or 100% amino acid sequence identity to the motifs 1-4 of a known Cas9/Csn1 amino acid sequence.

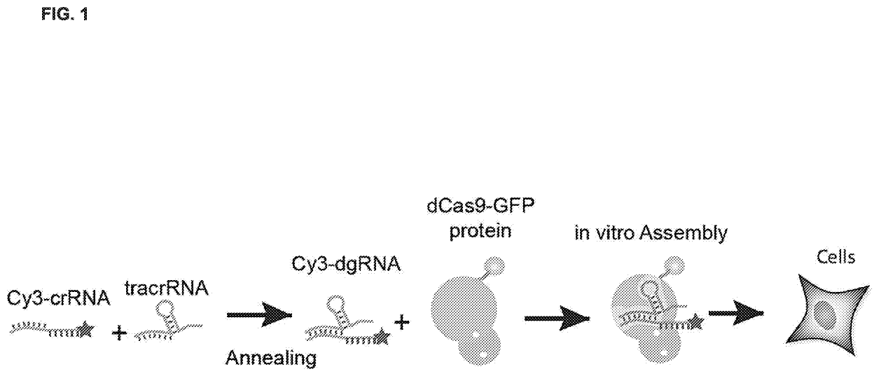

A number of bacteria express Cas9 protein variants. The Cas9 from Streptococcus pyogenes is presently the most commonly used; some of the other Cas9 proteins have high levels of sequence identity with the S. pyogenes Cas9 and use the same guide RNAs. Others are more diverse, use different gRNAs, and recognize different PAM sequences as well (the 2-5 nucleotide sequence specified by the protein which is adjacent to the sequence specified by the RNA). Chylinski et al. classified Cas9 proteins from a large group of bacteria (RNA Biology 10:5, 1-12; 2013, incorporated herein by reference), and a large number of Cas9 proteins are listed in supplementary FIG. 1 and supplementary table 1 thereof, which are incorporated by reference herein. Additional Cas9 proteins are described in Esvelt et al., Nat Methods. 2013 November; 10(11)1116-21 and Fonfara et al., “Phylogeny of Cas9 determines functional exchangeability of dual-RNA and Cas9 among orthologous type II CRISPR-Cas systems.” Nucleic Acids Res. 42: 2577-90 (2014), each of which is incorporated herein by reference.

Cas9, and thus dCas9, molecules of a variety of species find use in the technology described herein. While the S. pyogenes and S. thermophilus Cas9 molecules are widely used, Cas9 molecules of, derived from, or based on the Cas9 proteins of other species listed herein find use in embodiments of the technology. Accordingly, the technology provides for the replacement of S. pyogenes and S. thermophilus Cas9 and dCas9 molecules with Cas9 and dCas9 molecules from the other species can replace them, e.g.:

| GenBank Acc No. | Bacterium |
|---|---|
| 303229466 | Veillonella atypica ACS-134-V-Col7a |
| 34762592 | Fusobacterium nucleatum subsp. vincentii |
| 374307738 | Filifactor alocis ATCC 35896 |
| 320528778 | Solobacterium moorei F0204 |
| 291520705 | Coprococcus catus GD-7 |
| 42525843 | Treponema denticola ATCC 35405 |
| 304438954 | Peptoniphilus duerdenii ATCC BAA-1640 |
| 224543312 | Catenibacterium mitsuokai DSM 15897 |
| 24379809 | Streptococcus mutans UA159 |
| 15675041 | Streptococcus pyogenes SF370 |
| 16801805 | Listeria innocua Clip11262 |
| 116628213 | Streptococcus thermophilus LMD-9 |
| 323463801 | Staphylococcus pseudintermedius ED99 |
| 352684361 | Acidaminococcus intestini RyC-MR95 |
| 302336020 | Olsenella uli DSM 7084 |
| 366983953 | Oenococcus kitaharae DSM 17330 |
| 310286728 | Bifidobacterium bifidum S17 |
| 258509199 | Lactobacillus rhamnosus GG |
| 300361537 | Lactobacillus gasseri JV-V03 |
| 169823755 | Finegoldia magna ATCC 29328 |
| 47458868 | Mycoplasma mobile 163K |
| 284931710 | Mycoplasma gallisepticum str. F |
| 363542550 | Mycoplasma ovipneumoniae SC01 |
| 384393286 | Mycoplasma canis PG 14 |
| 71894592 | Mycoplasma synoviae 53 |
| 238924075 | Eubacterium rectale ATCC 33656 |
| 116627542 | Streptococcus thermophilus LMD-9 |
| 315149830 | Enterococcus faecalis TX0012 |
| 315659848 | Staphylococcus lugdunensis M23590 |
| 160915782 | Eubacterium dolichum DSM 3991 |
| 336393381 | Lactobacillus coryniformis subsp. torquens |
| 310780384 | Ilyobacter polytropus DSM 2926 |
| 325677756 | Ruminococcus albus 8 |
| 187736489 | Akkermansia muciniphila ATCC BAA-835 |
| 117929158 | Acidothermus cellulolyticus 11B |
| 189440764 | Bifidobacterium longum DJO10A |
| 283456135 | Bifidobacterium dentium Bd1 |
| 38232678 | Corynebacterium diphtheriae NCTC 13129 |
| 187250660 | Elusimicrobium minutum Pei191 |
| 319957206 | Nitratifractor salsuginis DSM 16511 |
| 325972003 | Sphaerochaeta globus str. Buddy |
| 261414553 | Fibrobacter succinogenes subsp. succinogenes |
| 60683389 | Bacteroides fragilis NCTC 9343 |
| 256819408 | Capnocytophaga ochracea DSM 7271 |
| 90425961 | Rhodopseudomonas palustris BisB18 |
| 373501184 | Prevotella micans F0438 |
| 294674019 | Prevotella ruminicola 23 |
| 365959402 | Flavobacterium columnare ATCC 49512 |
| 312879015 | Aminomonas paucivorans DSM 12260 |
| 83591793 | Rhodospirillum rubrum ATCC 11170 |
| 294086111 | Candidatus Puniceispirillum marinum IMCC1322 |
| 121608211 | Verminephrobacter eiseniae EF01-2 |
| 344171927 | Ralstonia syzygii R24 |
| 159042956 | Dinoroseobacter shibae DFL 12 |
| 288957741 | Azospirillum sp-B510 |
| 92109262 | Nitrobacter hamburgensis X14 |
| 148255343 | Bradyrhizobium sp-BTAi1 |
| 34557790 | Wolinella succinogenes DSM 1740 |
| 218563121 | Campylobacter jejuni subsp. jejuni |
| 291276265 | Helicobacter mustelae 12198 |
| 229113166 | Bacillus cereus Rock1-15 |
| 222109285 | Acidovorax ebreus TPSY |
| 189485225 | uncultured Termite group 1 |
| 182624245 | Clostridium perfringens D str. |
| 220930482 | Clostridium cellulolyticum H10 |
| 154250555 | Parvibaculum lavamentivorans DS-1 |
| 257413184 | Roseburia intestinalis L1-82 |
| 218767588 | Neisseria meningitidis Z2491 |
| 15602992 | Pasteurella multocida subsp. multocida |
| 319941583 | Sutterella wadsworthensis 3 1 |
| 254447899 | gamma proteobacterium HTCC5015 |
| 54296138 | Legionella pneumophila str. Paris |
| 331001027 | Parasutterella excrementihominis YIT 11859 |
| 34557932 | Wolinella succinogenes DSM 1740 |
| 118497352 | Francisella novicida U112 |

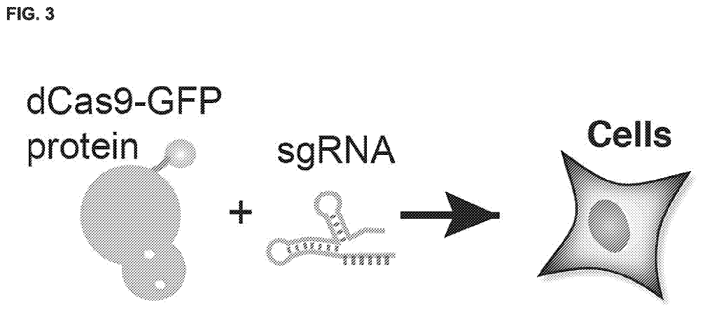

See also U.S. Pat. App. Pub. No. 20170051312 at FIGS. 3, 4, 5, incorporated herein by reference.

The technology described herein encompasses the use of a dCas9 derived from any Cas9 protein (e.g., as listed above) and their corresponding guide RNAs or other guide RNAs that are compatible. The Cas9 from Streptococcus thermophilus LMD-9 CRISPR1 system has been shown to function in human cells (see, e.g., Cong et al. (2013) Science 339: 819, incorporated herein by reference). Additionally, Jinek showed in vitro that Cas9 orthologs from S. thermophilus and L. innocua, can be guided by a dual S. pyogenes gRNA to cleave target plasmid DNA.

In some embodiments, the present technology comprises the Cas9 protein from S. pyogenes, either as encoded in bacteria or codon-optimized for expression in mammalian cells, containing mutations at D10, E762, H983, or D986 and H840 or N863, e.g., D 10A/D10N and H840A/H840N/H840Y, to render the nuclease portion of the protein catalytically inactive; substitutions at these positions are, in some embodiments, alanine (Nishimasu (2014) Cell 156: 935-949, incorporated herein by reference) or, in some embodiments, other residues, e.g., glutamine, asparagine, tyrosine, serine, or aspartate, e.g., E762Q, H983N, H983Y, D986N, N863D, N863S, or N863H. In some embodiments, the dCas9 is produced by one or more conservative substitutions produced in a Cas9 protein. The sequence of one S. pyogenes dCas9 protein that finds use in the technology provided herein is described in U.S. Pat. App. Pub. No. US20160010076, which is incorporated herein by reference in its entirety.

For example, in some embodiments, the dCas9 used herein is at least about 50% identical to the sequence of S. pyogenes Cas9, e.g., at least 50% identical to the following sequence of dCas9 comprising the D10A and H840A substitutions (SEQ ID NO: 1).

```
Met Asp Lys Lys Tyr Ser Ile Gly Leu Ala Ile Gly Thr Asn Ser Val
1 5 10 15
Gly Trp Ala Val Ile Thr Asp Glu Tyr Lys Val Pro Ser Lys Lys Phe
                        20 25 30
Lys Val Leu Gly Asn Thr Asp Arg His Ser Ile Lys Lys Asn Leu Ile
                35 40 45
Gly Ala Leu Leu Phe Asp Ser Gly Glu Thr Ala Glu Ala Thr Arg Leu
        50 55 60
Lys Arg Thr Ala Arg Arg Arg Tyr Thr Arg Arg Lys Asn Arg Ile Cys
65 70 75 80
Tyr Leu Gln Glu Ile Phe Ser Asn Glu Met Ala Lys Val Asp Asp Ser
                                85 90 95
Phe Phe His Arg Leu Glu Glu Ser Phe Leu Val Glu Glu Asp Lys Lys
                        100 105 110
His Glu Arg His Pro Ile Phe Gly Asn Ile Val Asp Glu Val Ala Tyr
                115 120 125
His Glu Lys Tyr Pro Thr Ile Tyr His Leu Arg Lys Lys Leu Val Asp
        130 135 140
Ser Thr Asp Lys Ala Asp Leu Arg Leu Ile Tyr Leu Ala Leu Ala His
145 150 155 160
Met Ile Lys Phe Arg Gly His Phe Leu Ile Glu Gly Asp Leu Asn Pro
                                165 170 175
Asp Asn Ser Asp Val Asp Lys Leu Phe Ile Gln Leu Val Gln Thr Tyr
                        180 185 190
Asn Gln Leu Phe Glu Glu Asn Pro Ile Asn Ala Ser Gly Val Asp Ala
                195 200 205
Lys Ala Ile Leu Ser Ala Arg Leu Ser Lys Ser Arg Arg Leu Glu Asn
        210 215 220
Leu Ile Ala Gln Leu Pro Gly Glu Lys Lys Asn Gly Leu Phe Gly Asn
225 230 235 240
Leu Ile Ala Leu Ser Leu Gly Leu Thr Pro Asn Phe Lys Ser Asn Phe
                                245 250 255
Asp Leu Ala Glu Asp Ala Lys Leu Gln Leu Ser Lys Asp Thr Tyr Asp
                        260 265 270
Asp Asp Leu Asp Asn Leu Leu Ala Gln Ile Gly Asp Gln Tyr Ala Asp
                275 280 285
Leu Phe Leu Ala Ala Lys Asn Leu Ser Asp Ala Ile Leu Leu Ser Asp
        290 295 300
Ile Leu Arg Val Asn Thr Glu Ile Thr Lys Ala Pro Leu Ser Ala Ser
305 310 315 320
Met Ile Lys Arg Tyr Asp Glu His His Gln Asp Leu Thr Leu Leu Lys
                                325 330 335
Ala Leu Val Arg Gln Gln Leu Pro Glu Lys Tyr Lys Glu Ile Phe Phe
                        340 345 350
Asp Gln Ser Lys Asn Gly Tyr Ala Gly Tyr Ile Asp Gly Gly Ala Ser
                355 360 365
Gln Glu Glu Phe Tyr Lys Phe Ile Lys Pro Ile Leu Glu Lys Met Asp
        370 375 380
Gly Thr Glu Glu Leu Leu Val Lys Leu Asn Arg Glu Asp Leu Leu Arg
385 390 395 400
Lys Gln Arg Thr Phe Asp Asn Gly Ser Ile Pro His Gln Ile His Leu
                                405 410 415
Gly Glu Leu His Ala Ile Leu Arg Arg Gln Glu Asp Phe Tyr Pro Phe
                        420 425 430
Leu Lys Asp Asn Arg Glu Lys Ile Glu Lys Ile Leu Thr Phe Arg Ile
                435 440 445
Pro Tyr Tyr Val Gly Pro Leu Ala Arg Gly Asn Ser Arg Phe Ala Trp
        450 455 460
Met Thr Arg Lys Ser Glu Glu Thr Ile Thr Pro Trp Asn Phe Glu Glu
465 470 475 480
Val Val Asp Lys Gly Ala Ser Ala Gln Ser Phe Ile Glu Arg Met Thr
                                485 490 495
Asn Phe Asp Lys Asn Leu Pro Asn Glu Lys Val Leu Pro Lys His Ser
                        500 505 510
Leu Leu Tyr Glu Tyr Phe Thr Val Tyr Asn Glu Leu Thr Lys Val Lys
                515 520 525
Tyr Val Thr Glu Gly Met Arg Lys Pro Ala Phe Leu Ser Gly Glu Gln
        530 535 540
Lys Lys Ala Ile Val Asp Leu Leu Phe Lys Thr Asn Arg Lys Val Thr
545 550 555 560
Val Lys Gln Leu Lys Glu Asp Tyr Phe Lys Lys Ile Glu Cys Phe Asp
                                565 570 575
Ser Val Glu Ile Ser Gly Val Glu Asp Arg Phe Asn Ala Ser Leu Gly
                        580 585 590
Thr Tyr His Asp Leu Leu Lys Ile Ile Lys Asp Lys Asp Phe Leu Asp
                595 600 605
Asn Glu Glu Asn Glu Asp Ile Leu Glu Asp Ile Val Leu Thr Leu Thr
        610 615 620
Leu Phe Glu Asp Arg Glu Met Ile Glu Glu Arg Leu Lys Thr Tyr Ala
625 630 635 640
His Leu Phe Asp Asp Lys Val Met Lys Gln Leu Lys Arg Arg Arg Tyr
                                645 650 655
Thr Gly Trp Gly Arg Leu Ser Arg Lys Leu Ile Asn Gly Ile Arg Asp
                        660 665 670
Lys Gln Ser Gly Lys Thr Ile Leu Asp Phe Leu Lys Ser Asp Gly Phe
                675 680 685
Ala Asn Arg Asn Phe Met Gln Leu Ile His Asp Asp Ser Leu Thr Phe
        690 695 700
Lys Glu Asp Ile Gln Lys Ala Gln Val Ser Gly Gln Gly Asp Ser Leu
705 710 715 720
His Glu His Ile Ala Asn Leu Ala Gly Ser Pro Ala Ile Lys Lys Gly
                                725 730 735
Ile Leu Gln Thr Val Lys Val Val Asp Glu Leu Val Lys Val Met Gly
                        740 745 750
Arg His Lys Pro Glu Asn Ile Val Ile Glu Met Ala Arg Glu Asn Gln
                755 760 765
Thr Thr Gln Lys Gly Gln Lys Asn Ser Arg Glu Arg Met Lys Arg Ile
        770 775 780
Glu Glu Gly Ile Lys Glu Leu Gly Ser Gln Ile Leu Lys Glu His Pro
785 790 795 800
Val Glu Asn Thr Gln Leu Gln Asn Glu Lys Leu Tyr Leu Tyr Tyr Leu
                                805 810 815
Gln Asn Gly Arg Asp Met Tyr Val Asp Gln Glu Leu Asp Ile Asn Arg
                        820 825 830
Leu Ser Asp Tyr Asp Val Asp Ala Ile Val Pro Gln Ser Phe Leu Lys
                835 840 845
Asp Asp Ser Ile Asp Asn Lys Val Leu Thr Arg Ser Asp Lys Asn Arg
        850 855 860
Gly Lys Ser Asp Asn Val Pro Ser Glu Glu Val Val Lys Lys Met Lys
865 870 875 880
Asn Tyr Trp Arg Gln Leu Leu Asn Ala Lys Leu Ile Thr Gln Arg Lys
                                885 890 895
Phe Asp Asn Leu Thr Lys Ala Glu Arg Gly Gly Leu Ser Glu Leu Asp
                        900 905 910
Lys Ala Gly Phe Ile Lys Arg Gln Leu Val Glu Thr Arg Gln Ile Thr
                915 920 925
Lys His Val Ala Gln Ile Leu Asp Ser Arg Met Asn Thr Lys Tyr Asp
        930 935 940
Glu Asn Asp Lys Leu Ile Arg Glu Val Lys Val Ile Thr Leu Lys Ser
945 950 955 960
Lys Leu Val Ser Asp Phe Arg Lys Asp Phe Gln Phe Tyr Lys Val Arg
                                965 970 975
Glu Ile Asn Asn Tyr His His Ala His Asp Ala Tyr Leu Asn Ala Val
                980 985 990
Val Gly Thr Ala Leu Ile Lys Lys Tyr Pro Lys Leu Glu Ser Glu Phe
                995 1000 1005
Val Tyr Gly Asp Tyr Lys Val Tyr Asp Val Arg Lys Met Ile Ala
        1010 1015 1020
Lys Ser Glu Gln Glu Ile Gly Lys Ala Thr Ala Lys Tyr Phe Phe
        1025 1030 1035
Tyr Ser Asn Ile Met Asn Phe Phe Lys Thr Glu Ile Thr Leu Ala
        1040 1045 1050
Asn Gly Glu Ile Arg Lys Arg Pro Leu Ile Glu Thr Asn Gly Glu
        1055 1060 1065
Thr Gly Glu Ile Val Trp Asp Lys Gly Arg Asp Phe Ala Thr Val
        1070 1075 1080
Arg Lys Val Leu Ser Met Pro Gln Val Asn Ile Val Lys Lys Thr
        1085 1090 1095
Glu Val Gln Thr Gly Gly Phe Ser Lys Glu Ser Ile Leu Pro Lys
        1100 1105 1110
Arg Asn Ser Asp Lys Leu Ile Ala Arg Lys Lys Asp Trp Asp Pro
        1115 1120 1125
Lys Lys Tyr Gly Gly Phe Asp Ser Pro Thr Val Ala Tyr Ser Val
        1130 1135 1140
Leu Val Val Ala Lys Val Glu Lys Gly Lys Ser Lys Lys Leu Lys
        1145 1150 1155
Ser Val Lys Glu Leu Leu Gly Ile Thr Ile Met Glu Arg Ser Ser
        1160 1165 1170
Phe Glu Lys Asn Pro Ile Asp Phe Leu Glu Ala Lys Gly Tyr Lys
        1175 1180 1185
Glu Val Lys Lys Asp Leu Ile Ile Lys Leu Pro Lys Tyr Ser Leu
        1190 1195 1200
Phe Glu Leu Glu Asn Gly Arg Lys Arg Met Leu Ala Ser Ala Gly
        1205 1210 1215
Glu Leu Gln Lys Gly Asn Glu Leu Ala Leu Pro Ser Lys Tyr Val
        1220 1225 1230
Asn Phe Leu Tyr Leu Ala Ser His Tyr Glu Lys Leu Lys Gly Ser
        1235 1240 1245
Pro Glu Asp Asn Glu Gln Lys Gln Leu Phe Val Glu Gln His Lys
        1250 1255 1260
His Tyr Leu Asp Glu Ile Ile Glu Gln Ile Ser Glu Phe Ser Lys
        1265 1270 1275
Arg Val Ile Leu Ala Asp Ala Asn Leu Asp Lys Val Leu Ser Ala
        1280 1285 1290
Tyr Asn Lys His Arg Asp Lys Pro Ile Arg Glu Gln Ala Glu Asn
        1295 1300 1305
Ile Ile His Leu Phe Thr Leu Thr Asn Leu Gly Ala Pro Ala Ala
        1310 1315 1320
Phe Lys Tyr Phe Asp Thr Thr Ile Asp Arg Lys Arg Tyr Thr Ser
        1325 1330 1335
Thr Lys Glu Val Leu Asp Ala Thr Leu Ile His Gln Ser Ile Thr
        1340 1345 1350
Gly Leu Tyr Glu Thr Arg Ile Asp Leu Ser Gln Leu Gly Gly Asp
        1355 1360 1365
```

In some embodiments, the technology comprises use of a nucleotide sequence that is approximately 50%, 55%, 60%, 65%, 70%, 75%, 80%, 85%, 90%, 95%, 99% or 100% identical to a nucleotide sequence that encodes a protein described by SEQ ID NO: 1.

In some embodiments, the dCas9 used herein is at least about 50% identical to the sequence of the catalytically inactive S. pyogenes Cas9, e.g., at least 50%, 55%, 60%, 65%, 70%, 75%, 80%, 85%, 90%, 95%, 99% or 100% identical to SEQ ID NO: 1, wherein the mutations at D10 and H840, e.g., D10A/D10N and H840A/H840N/H840Y are maintained.

In some embodiments, the polypeptide (e.g., the RNA-guided nuclease) of the RNP is a Cas protein, CRISPR enzyme, or Cas-like protein. “Cas protein” and “CRISPR enzyme” and “Cas-like protein”, as used herein, includes polypeptides, enzymatic activities, and polypeptides having activities similar to proteins known in the art as, or encoded by genes known in the art as, e.g., Cast, Cas1B, Cas2, Cas3, Cas4, Cas5, Cash, Cas7, Cas8, Cas9 (also known as Csn1 and Csx12), Cas10, Cas13, Csyl, Csy2, Csy3, Cse1, Cse2, Cscl, Csc2, Csa5, Csn2, Csm2, Csm3, Csm4, Csm5, Csm6, Cmrl, Cmr3, Cmr4, Cmr5, Cmr6, Csb1, Csb2, Csb3, Csx17, Csx14, Csx10, Csx16, CsaX, Csx3, Csxl, Csx15, Csfl, Csf2, Csf3, Csf4, Cpf1, C2c1, C2c2, homologs thereof, or modified versions thereof, e.g., including nuclease-deficient versions (“d” versions) of any of these Cas proteins, CRISPR enzymes, and/or Cas-like proteins known in the art such as dCas9.

In embodiments, the technology comprises use of a polypeptide (e.g., a Type V/Type VI protein) such as Cpf1 or C2c1 or C2c2 and homologs and orthologs of a Type V/Type VI protein such as Cpf1 or C2c1 or C2c2. Embodiments encompass Cpf1, modified Cpf1, chimeric, and deactivated/inactivated Cpf1, and CRISPR systems related to Cpf1, modified Cpf1, chimeric, and deactivated/inactivated Cpf1. In some embodiments, the polypeptide (e.g., a Type V/Type VI protein) such as Cpf1 or C2c1 or C2c2 is from a genus that is, e.g., Streptococcus, Campylobacter, Nitratifractor, Staphylococcus, Parvibaculum, Roseburia, Neisseria, Gluconacetobacter, Azospirillum, Sphaerochaeta, Lactobacillus, Eubacterium, Corynebacter, Carnobacterium, Rhodobacter; Listeria, Paludibacter, Clostridium, Lachnospiraceae, Clostridiaridium, Leptotrichia, Francisella, Legionella, Alicyclobacillus, Methanomethyophilus, Porphyromonas, Prevotella, Bacteroidetes, Helcococcus, Letospira, Desulfovibrio, Desulfonatronum, Opitutaceae, Tuberibacillus, Bacillus, Brevibacilus, Methylobacterium, or Acidaminococcus. In some embodiments, the polypeptide (e.g., a Type V/Type VI protein) such as Cpf1 or C2c1 or C2c2 is from an organism that is, e.g., S. mutans, S. agalactiae, S. equisimilis, S. sanguinis, S. pneumonia; C. jejuni, C. coli; N. salsuginis, N. tergarcus; S. auricularis; S. carnosus; N. meningitides, N. gonorrhoeae; L. monocytogenes, L. ivanovii; C. botulinum, C. difficile, C. tetani, or C. sordellii. See, e.g., U.S. Pat. No. 9,790,490, incorporated herein by reference in its entirety.

As used herein, the term “nuclease-deficient” refers to a protein comprising reduced nuclease activity, minimized nuclease activity, undetectable nuclease activity, and/or having no nuclease activity, e.g., as a result of amino acid substitutions that reduce, minimize, and/or eliminate the nuclease activity of a protein.

In some embodiments, any differences from SEQ ID NO: 1 are in non-conserved regions, as identified by sequence alignment of sequences set forth in Chylinski et al., RNA Biology 10:5, 1-12; 2013 (e.g., in supplementary FIG. 1 and supplementary table 1 thereof); Esvelt et al., Nat Methods. 2013 November; 10(11)1116-21 and Fonfara et al., Nucl. Acids Res. (2014) 42 (4): 2577-2590, each of which is incorporated herein by reference, and wherein the mutations at D10 and H840, e.g., D10A/D 10N and H840A/H840N/H840Y are maintained.

Thus, in some cases, the polypeptide of the RNP is a naturally-occurring polypeptide. In some embodiments, the polypeptide of the RNP is not a naturally-occurring polypeptide (e.g., a chimeric polypeptide, a naturally-occurring polypeptide that is modified, e.g., by one or more amino acid substitutions produced by an engineered nucleic acid comprising one or more nucleotide substitutions, deletions, insertions).

Choosing, designing, synthesizing, and analyzing nucleotide sequences and amino acid sequences (e.g., of the polypeptide and RNA components of an RNP complex as described herein) often comprise use of sequence alignment methods to identify similarities and differences in two or more nucleotide sequences or amino acid sequences. To determine the percent identity of two sequences, the sequences are aligned for optimal comparison purposes (gaps are introduced in one or both of a first and a second amino acid or nucleic acid sequence as required for optimal alignment, and non-homologous sequences can be disregarded for comparison purposes). The length of a reference sequence aligned for comparison purposes is at least 50% (in some embodiments, about 50%, 55%, 60%, 65%, 70%, 75%, 85%, 90%, 95%, or 100% of the length of the reference sequence). The nucleotides or residues at corresponding positions are then compared. When a position in the first sequence is occupied by the same nucleotide or residue as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm In some embodiments, the percent identity between two amino acid sequences is determined using the Needleman and Wunsch ((1970) J. Mol. Biol. 48:444-453, incorporated herein by reference) algorithm, which has been incorporated into the GAP program in the GCG software package, e.g., using a Blosum 62 scoring matrix with a gap penalty of 12, a gap extend penalty of 4, and a frameshift gap penalty of 5. Other methods are known in the art, e.g., as discussed elsewhere herein.

In some embodiments, the RNP comprises a protein that is a Cas9 or Cas9 derivative, e.g., a dCas9. Thus, in some embodiments, the protein is a Type II Cas9 protein, a nuclease-deactivated Cas9 (dCas9), e.g., comprising substitutions to reduce, minimize, or eliminate the nuclease activity. In some embodiments, a dCas9 is a Streptococcus pyogenes Cas9 comprising one or more substitutions such as D10A and H841A. In some embodiments, the dCas9 has been engineered to partially remove the nuclease domain (e.g., (Cas9 nickase; see, e.g., Nature Methods 11; 399-402 (2014), incorporated herein by reference). In some embodiments, the RNP protein is a protein from CRISPR system other than the S. pyogenes system, e.g., a Type V Cpf1, C2c1, C2c2, C2c3 proteins and derivatives thereof.

In some embodiments, the polypeptide of the RNP is a chimeric or fusion polypeptide, e.g., a polypeptide that comprises two or more functional domains. For example, in some embodiments a chimeric polypeptide interacts with (e.g., binds to) an RNA to form an RNP (described above). The RNA guides the polypeptide to a target sequence within target DNA (e.g. a chromosomal sequence or an extrachromosomal sequence, e.g. an episomal sequence, a minicircle sequence, a mitochondrial sequence, a chloroplast sequence, etc.). Thus, in some embodiments a chimeric polypeptide binds target DNA.

A chimeric polypeptide comprises at least two portions, e.g., an RNA binding portion and an “activity” portion (e.g., a label). A chimeric polypeptide comprises amino acid sequences that are derived from at least two different polypeptides. A chimeric polypeptide can comprise modified and/or naturally occurring polypeptide sequences (e.g., a first amino acid sequence from a modified or unmodified Cas9/Csn1 protein; and a second amino acid sequence other than the Cas9/Csn1 protein).

In some embodiments, the RNA-binding portion of a chimeric polypeptide is a naturally-occurring polypeptide. In some embodiments, the RNA-binding portion of a chimeric polypeptide is not a naturally-occurring molecule (e.g., modified with respect to a naturally-occurring polypeptide by, e.g., substitution, deletion, insertion). In some embodiments, naturally-occurring RNA-binding portions of interest are derived from polypeptides known in the art, e.g., discussed herein (e.g., Cas9 and similar polypeptides).

In some embodiments, the RNA-binding portion of a chimeric polypeptide comprises an amino acid sequence having at least about 75%, at least about 80%, at least about 85%, at least about 90%, at least about 95%, at least about 98%, at least about 99%, or 100% amino acid sequence identity to the RNA-binding portion of a polypeptide described herein.

In some embodiments, the chimeric polypeptide comprises an amino acid sequence having at least about 75%, at least about 80%, at least about 85%, at least about 90%, at least about 95%, at least about 99%, or 100% amino acid sequence identity to a portion of a Cas9 amino acid sequence provided herein.

In addition to the RNA-binding portion, the chimeric polypeptide comprises an “activity portion.” In some embodiments, the activity portion of a chimeric polypeptide comprises the naturally-occurring activity portion of a site-directed modifying polypeptide (e.g., a Cas9/Csn1 endonuclease). In other embodiments, the activity portion of a chimeric polypeptide comprises a modified amino acid sequence (e.g., substitution, deletion, insertion) of a naturally-occurring activity portion of a site-directed modifying polypeptide. The activity portion of a chimeric polypeptide is variable and may comprise any heterologous polypeptide sequence that may be useful in the methods disclosed herein. For example, in some embodiments, the activity portion comprises a label or provides a label function as described herein.

In some embodiments, a chimeric polypeptide comprises: (i) an RNA-binding portion that interacts with a DNA-targeting RNA, wherein the DNA-targeting RNA comprises a nucleotide sequence that is complementary to a sequence in a target DNA; and (ii) an activity portion that exhibits site-directed enzymatic activity (e.g., activity for DNA methylation, activity for DNA cleavage, activity for histone acetylation, activity for histone methylation, etc.), wherein the site of enzymatic activity is determined by the DNA-targeting RNA. In other embodiments, a chimeric polypeptide comprises: (i) an RNA-binding portion that interacts with a DNA-targeting RNA, wherein the DNA-targeting RNA comprises a nucleotide sequence that is complementary to a sequence in a target DNA; and (ii) an activity portion that modulates transcription within the target DNA (e.g., to increase or decrease transcription), wherein the site of modulated transcription within the target DNA is determined by the DNA-targeting RNA. In some embodiments, the activity portion of a chimeric polypeptide has enzymatic activity that modifies target DNA (e.g., nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity or glycosylase activity). In some embodiments, the activity portion of a chimeric polypeptide has enzymatic activity (e.g., methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity or demyristoylation activity) that modifies a polypeptide associated with target DNA (e.g., a histone). In some embodiments, the activity portion of a chimeric polypeptide exhibits enzymatic activity (described above). In other embodiments, the activity portion of a chimeric polypeptide modulates transcription of the target DNA (described above). The activity portion of a chimeric polypeptide is variable and may comprise any heterologous polypeptide sequence that may be useful in the methods disclosed herein.

In some embodiments, the activity portion comprises a fluorescent protein (e.g., a green fluorescent protein (GFP), a modified derivative of GFP (e.g., a GFP comprising S65T, an enhanced GFP or “EGFP” (e.g., comprising F64L)), or others known in the art such as, e.g., blue fluorescent protein (e.g., EBFP, EBFP2, Azurite, mKalama1), cyan fluorescent protein (e.g., ECFP, Cerulean, CyPet, mTurquoise2), and yellow fluorescent protein and yellow fluorescent protein derivatives (e.g., YFP, Citrine, Venus, YPet). In some embodiments, the activity portion comprises a HALOTAG protein that forms a complex with a fluorescent dye as known in the art and as described herein.

The technology provided herein comprises embodiments related to a RNP for imaging a nucleic acid, cell, or tissue. In particular, the RNP comprises a polypeptide (e.g., a Cas9, a dCas9, or a similar polypeptide) and an RNA (e.g., a gRNA, e.g., a sgRNA, a dgRNA) that directs the RNP to a specific target sequence, in a nucleic acid, chromosome, etc.

A gRNA comprises a first segment (also referred to herein as a “DNA-targeting segment” or a “DNA-targeting sequence”) and a second segment (also referred to herein as a “protein-binding segment” or a “protein-binding sequence”).

The DNA-targeting segment of a gRNA comprises a nucleotide sequence that is complementary to a sequence in a target DNA. In other words, the DNA-targeting segment of a gRNA interacts with a target DNA in a sequence-specific manner via hybridization (e.g., complementary base pairing). As such, the nucleotide sequence of the DNA targeting segment may vary and determines the location within the target DNA that the DNA targeting RNA and the target DNA will interact. The DNA-targeting segment of a gRNA can be modified (e.g., by genetic engineering) to hybridize to any desired sequence within a target DNA.

The DNA-targeting segment (e.g., comprising the DNA-targeting sequence and, in some embodiments, additional nucleic acid) can have a length of from about 8 nucleotides to about 100 nucleotides. For example, the DNA-targeting segment can have a length of from about 12 nucleotides (nt) to about 80 nt, from about 12 nt to about 50 nt, from about 12 nt to about 40 nt, from about 12 nt to about 30 nt, from about 12 nt to about 25 nt, from about 12 nt to about 20 nt, or from about 12 nt to about 19 nt. For example, the DNA-targeting segment can have a length of from about 19 nt to about 20 nt, from about 19 nt to about 25 nt, from about 19 nt to about 30 nt, from about 19 nt to about 35 nt, from about 19 nt to about 40 nt, from about 19 nt to about 45 nt, from about 19 nt to about 50 nt, from about 19 nt to about 60 nt, from about 19 nt to about 70 nt, from about 19 nt to about 80 nt, from about 19 nt to about 90 nt, from about 19 nt to about 100 nt, from about 20 nt to about 25 nt, from about 20 nt to about 30 nt, from about 20 nt to about 35 nt, from about 20 nt to about 40 nt, from about 20 nt to about 45 nt, from about 20 nt to about 50 nt, from about 20 nt to about 60 nt, from about 20 nt to about 70 nt, from about 20 nt to about 80 nt, from about 20 nt to about 90 nt, or from about 20 nt to about 100 nt.

In some embodiments, the nucleotide sequence (the DNA-targeting sequence) of the DNA-targeting segment that is complementary to a nucleotide sequence (target sequence) of the target DNA can have a length at least about 12 nt. For example, the DNA-targeting sequence of the DNA-targeting segment that is complementary to a target sequence of the target DNA can have a length at least about 12 nt, at least about 15 nt, at least about 18 nt, at least about 19 nt, at least about 20 nt, at least about 25 nt, at least about 30 nt, at least about 35 nt or at least about 40 nt. For example, the DNA-targeting sequence of the DNA-targeting segment that is complementary to a target sequence of the target DNA can have a length of from about 12 nucleotides (nt) to about 80 nt, from about 12 nt to about 50 nt, from about 12 nt to about 45 nt, from about 12 nt to about 40 nt, from about 12 nt to about 35 nt, from about 12 nt to about 30 nt, from about 12 nt to about 25 nt, from about 12 nt to about 20 nt, from about 12 nt to about 19 nt, from about 19 nt to about 20 nt, from about 19 nt to about 25 nt, from about 19 nt to about 30 nt, from about 19 nt to about 35 nt, from about 19 nt to about 40 nt, from about 19 nt to about 45 nt, from about 19 nt to about 50 nt, from about 19 nt to about 60 nt, from about 20 nt to about 25 nt, from about 20 nt to about 30 nt, from about 20 nt to about 35 nt, from about 20 nt to about 40 nt, from about 20 nt to about 45 nt, from about 20 nt to about 50 nt, or from about 20 nt to about 60 nt. The nucleotide sequence (the DNA-targeting sequence) of the DNA-targeting segment that is complementary to a nucleotide sequence (target sequence) of the target DNA can have a length at least about 12 nt.

In additional embodiments, the nucleotide sequence (the DNA-targeting sequence) of the DNA-targeting segment that is complementary to a nucleotide sequence (target sequence) of the target DNA can have a length of from about 8 nucleotides to about 30 nucleotides. For example, the DNA-targeting segment can have a length of from about 8 nucleotides (nt) to about 30 nt, from about 8 nt to about 30 nt, from about 8 nt to about 25 nt, from about 8 nt to about 20 nt, from about 8 nt to about 18 nt, from about 8 nt to about 15 nt, or from about 8 nt to about 12 nt, e.g., 8 nt, 9 nt, 10 nt, 11 nt, or 12 nt.

In some cases, the DNA-targeting sequence of the DNA-targeting segment that is complementary to a target sequence of the target DNA is 8-20 nucleotides in length. In some cases, the DNA-targeting sequence of the DNA-targeting segment that is complementary to a target sequence of the target DNA is 9-12 nucleotides in length.

The percent complementarity between the DNA-targeting sequence of the DNA-targeting segment and the target sequence of the target DNA can be at least 60% (e.g., at least 65%, at least 70%, at least 75%, at least 80%, at least 85%, at least 90%, at least 95%, at least 97%, at least 98%, at least 99%, or 100%). In some cases, the percent complementarity between the DNA-targeting sequence of the DNA-targeting segment and the target sequence of the target DNA is 100% over the seven contiguous 5′-most nucleotides of the target sequence of the complementary strand of the target DNA. In some cases, the percent complementarity between the DNA-targeting sequence of the DNA-targeting segment and the target sequence of the target DNA is at least 60% over about 20 contiguous nucleotides. In some cases, the percent complementarity between the DNA-targeting sequence of the DNA-targeting segment and the target sequence of the target DNA is 100% over the fourteen contiguous 5′-most nucleotides of the target sequence of the complementary strand of the target DNA and as low as 0% over the remainder. In such a case, the DNA-targeting sequence can be considered to be 14 nucleotides in length. In some cases, the percent complementarity between the DNA targeting sequence of the DNA-targeting segment and the target sequence of the target DNA is 100% over the seven contiguous 5′-most nucleotides of the target sequence of the complementary strand of the target DNA and as low as 0% over the remainder. In such a case, the DNA-targeting sequence can be considered to be 7 nucleotides in length.

The protein-binding segment of a gRNA interacts with a polypeptide, e.g., a Cas9, dCas9, or Cas9-like polypeptide. The gRNA guides the bound polypeptide to a specific nucleotide sequence within target DNA via the above mentioned DNA-targeting segment.

The protein-binding segment of a gRNA comprises two segments comprising nucleotide sequences that are complementary to one another. The complementary nucleotides of the protein-binding segment hybridize to form a double stranded RNA duplex.

A dgRNA comprises two separate RNA molecules. Each of the two RNA molecules of a dgRNA comprises a segment is complementary to one another such that the complementary nucleotides of the two RNA molecules hybridize to form the double stranded RNA duplex of the protein-binding segment.

In some embodiments, the duplex-forming segment of the activator-RNA is at least about 60% identical to one of the activator-RNA (tracrRNA) molecules set forth in U.S. Pat. App. Pub. No. 20170051312, incorporated herein by reference, as SEQ ID NOs: 431-562, or a complement thereof, over a segment of at least 8 contiguous nucleotides. For example, the duplex-forming segment of the activator-RNA (or the DNA encoding the duplex-forming segment of the activator-RNA) is at least about 60% identical, at least about 65% identical, at least about 70% identical, at least about 75% identical, at least about 80% identical, at least about 85% identical, at least about 90% identical, at least about 95% identical, at least about 98% identical, at least about 99% identical, or 100% identical, to one of the tracrRNA sequences set forth in U.S. Pat. App. Pub. No. 20170051312, incorporated herein by reference, as SEQ ID NOs: 431-562, or a complement thereof, over a segment of at least 8 contiguous nucleotides.

In some embodiments, the duplex-forming segment of the targeter-RNA is at least about 60% identical to one of the targeter-RNA (crRNA) sequences set forth in U.S. Pat. App. Pub. No. 20170051312, incorporated herein by reference, as SEQ ID NOs: 563-679, or a complement thereof, over a segment of at least 8 contiguous nucleotides. For example, the duplex-forming segment of the targeter-RNA (or the DNA encoding the duplex-forming segment of the targeter-RNA) is at least about 65% identical, at least about 70% identical, at least about 75% identical, at least about 80% identical, at least about 85% identical, at least about 90% identical, at least about 95% identical, at least about 98% identical, at least about 99% identical or 100% identical to one of the crRNA sequences set forth in U.S. Pat. App. Pub. No. 20170051312, incorporated herein by reference, as SEQ ID NOs: 563-679, or a complement thereof, over a segment of at least 8 contiguous nucleotides.

Non-limiting examples of nucleotide sequences that can be included in a two-molecule DNA targeting RNA (dgRNA) include either of the sequences set forth in U.S. Pat. App. Pub. No. 20170051312, incorporated herein by reference, as SEQ ID NOs: 431-562, or complements thereof pairing with any sequences set forth in U.S. Pat. App. Pub. No. 20170051312, incorporated herein by reference, as SEQ ID NOs: 563-679, or complements thereof that can hybridize to form a protein binding segment.

A single-molecule DNA-targeting RNA (sgRNA) comprises two segments of nucleotides (a targeter-RNA and an activator-RNA) that are complementary to one another, are covalently linked by intervening nucleotides (“linkers” or “linker nucleotides”), and hybridize to form the double stranded RNA duplex (dsRNA duplex) of the protein-binding segment, thus resulting in a stem-loop structure. The targeter-RNA and the activator-RNA can be covalently linked via the 3′ end of the targeter-RNA and the 5′ end of the activator-RNA. Alternatively, targeter-RNA and the activator-RNA can be covalently linked via the 5′ end of the targeter-RNA and the 3′ end of the activator-RNA.

The linker of a single-molecule DNA-targeting RNA can have a length of from about 3 nucleotides to about 100 nucleotides. For example, the linker can have a length of from about 3 nucleotides (nt) to about 90 nt, from about 3 nucleotides (nt) to about 80 nt, from about 3 nucleotides (nt) to about 70 nt, from about 3 nucleotides (nt) to about 60 nt, from about 3 nucleotides (nt) to about 50 nt, from about 3 nucleotides (nt) to about 40 nt, from about 3 nucleotides (nt) to about 30 nt, from about 3 nucleotides (nt) to about 20 nt or from about 3 nucleotides (nt) to about 10 nt. For example, the linker can have a length of from about 3 nt to about 5 nt, from about 5 nt to about 10 nt, from about 10 nt to about 15 nt, from about 15 nt to about 20 nt, from about 20 nt to about 25 nt, from about 25 nt to about 30 nt, from about 30 nt to about 35 nt, from about 35 nt to about 40 nt, from about 40 nt to about 50 nt, from about 50 nt to about 60 nt, from about 60 nt to about 70 nt, from about 70 nt to about 80 nt, from about 80 nt to about 90 nt, or from about 90 nt to about 100 nt. In some embodiments, the linker of a single molecule DNA-targeting RNA is 4 nt.

An exemplary single-molecule DNA-targeting RNA comprises two complementary segments of nucleotides that hybridize to form a dsRNA duplex. In some embodiments, one of the two complementary segments of nucleotides of the single-molecule DNA-targeting RNA (or the DNA encoding the segment) is at least about 60% identical to one of the activator-RNA (tracrRNA) molecules set forth in U.S. Pat. App. Pub. No. 20170051312, incorporated herein by reference, as SEQ ID NOs: 431-562, or a complement thereof, over a segment of at least 8 contiguous nucleotides. For example, one of the two complementary segments of nucleotides of the single-molecule DNA-targeting RNA (or the DNA encoding the segment) is at least about 65% identical, at least about 70% identical, at least about 75% identical, at least about 80% identical, at least about 85% identical, at least about 90% identical, at least about 95% identical, at least about 98% identical, at least about 99% identical or 100% identical to one of the tracrRNA sequences set forth in U.S. Pat. App. Pub. No. 20170051312, incorporated herein by reference, as SEQ ID NOs: 431-562, or a complement thereof, over a segment of at least 8 contiguous nucleotides.

In some embodiments, one of the two complementary segments of nucleotides of the single molecule DNA-targeting RNA (or the DNA encoding the segment) is at least about 60% identical to one of the targeter-RNA (crRNA) sequences set forth in U.S. Pat. App. Pub. No. 20170051312, incorporated herein by reference, as SEQ ID NOs: 563-679, or a complement thereof, over a segment of at least 8 contiguous nucleotides. For example, one of the two complementary segments of nucleotides of the single-molecule DNA-targeting RNA (or the DNA encoding the segment) is at least about 65% identical, at least about 70% identical, at least about 75% identical, at least about 80% identical, at least about 85% identical, at least about 90% identical, at least about 95% identical, at least about 98% identical, at least about 99% identical or 100% identical to one of the crRNA sequences set forth in U.S. Pat. App. Pub. No. 20170051312, incorporated herein by reference, as SEQ ID NOs: 563-679, or a complement thereof, over a stretch of at least 8 contiguous nucleotides.

With regard to both a sgRNA and a dgRNA, artificial sequences that share a wide range of identity (approximately at least 50% identity) with naturally occurring tracrRNAs and crRNAs function with Cas9 and dCas9 to deliver RNP to target nucleic acids with sequence specificity, particularly provided that the structure of the protein-binding domain of the DNA targeting RNA is conserved. Thus, information and modeling relating to RNA folding and RNA secondary structure of a naturally occurring protein-binding domain of a DNA-targeting RNA provides guidance to design artificial protein-binding domains (either in dgRNA or sgRNA). As a non-limiting example, a functional artificial DNA-targeting RNA may be designed based on the structure of the protein-binding segment of a naturally occurring DNA-targeting segment of an RNA (e.g., including the same or similar number of base pairs along the RNA duplex and including the same or similar “bulge” region as present in the naturally occurring RNA). Structures can readily be produced by one of ordinary skill in the art for any naturally occurring crRNA:tracrRNA pair from any species; thus, in some embodiments an artificial DNA-targeting-RNA is designed to mimic the natural structure for a given species when using the Cas9 or dCas9 (or a related Cas9 or dCas9) from that species. Thus, in some embodiments a suitable DNA-targeting RNA is an artificially designed RNA (non-naturally occurring) comprising a protein-binding domain that was designed to mimic the structure of a protein-binding domain of a naturally occurring DNA-targeting RNA. In exemplary embodiments, the protein-binding segment has a length of from about 10 nucleotides to about 100 nucleotides; e.g., the protein-binding segment has a length of from about 15 nucleotides (nt) to about 80 nt, from about 15 nt to about 50 nt, from about 15 nt to about 40 nt, from about 15 nt to about 30 nt or from about 15 nt to about 25 nt.

Nucleic acids can be analyzed and designed using a variety of computer tools, e.g., Vector NTI (Invitrogen) for nucleic acids and AlignX for comparative sequence analysis of proteins. Further, in silico modeling of RNA structure and folding can be performed using the Vienna RNA package algorithms and RNA secondary structures and folding models can be predicted with RNAfold and RNAcofold, respectively, and visualized with VARNA. See, e.g., Denman (1993), Biotechniques 15, 1090; Hofacker and Stadler (2006), Bioinformatics 22, 1172; and Darty and Ponty (2009), Bioinformatics 25, 1974, each of which is incorporated herein by reference.

Thus, as described herein, in some embodiments, the technology provides methods, systems, kits, compositions, uses, etc. comprising and/or comprising use of a RNP comprising a polypeptide and one or more RNAs. In some embodiments, the RNA comprises a segment (e.g., comprising 6-10 nucleotides, e.g., comprising 6, 7, 8, 9, or 10 nucleotides) that is complementary (e.g., at least 80, 81, 82, 83, 84, 85, 86, 87, 88, 89, 90, 91, 92, 93, 94, 95, 96, 97, 98, 98.5, 99, 99.1, 99.2, 99.3, 99.4, 99.5, 99.6, 99.7, 99.8, 99.9, or 100% complementary) to a nucleotide sequence in the target DNA.

In some embodiments, the RNA comprises a segment comprising a nucleotide sequence (e.g., a scaffold sequence, e.g., a sequence that interacts with (e.g., binds to) the polypeptide) that is at least 60% identical over at least 8 contiguous nucleotides to any one of the nucleotide sequences set forth in SEQ ID NOs: 431-682 (e.g., SEQ ID NOs: 431-562) of U.S. Pat. App. Pub. No. 20170051312, incorporated herein by reference. In some cases, the RNA comprises a nucleotide sequence (e.g., a scaffold sequence, e.g., a sequence that interacts with (e.g., binds to) the polypeptide) that is at least 60% identical over at least 8 contiguous nucleotides to any one of the nucleotide sequences set forth in SEQ ID NOs: 563-682 of U.S. Pat. App. Pub. No. 20170051312, incorporated herein by reference.

In some embodiments, the polypeptide comprises a segment comprising an amino acid sequence that is at least approximately 75% amino acid identical to amino acids 7-166 or 731-1003 of any of the amino acid sequences set forth as SEQ ID NOs: 1-256 and 795-1346 of U.S. Pat. App. Pub. No. 20170051312, incorporated herein by reference.

In some embodiments, the technology comprises use of a fluorescent RNA-guided nuclease (e.g., dCas9, dCpf1, Cas13) to target and/or label RNAs. In some embodiments, the technology relates to use of a labeled guide RNA that forms a complex (e.g., an RNP) with a RNA-guided nuclease to label and visualize RNA transcripts (e.g., an mRNA, a non-coding RNA (e.g., rRNA, microRNA, tRNA, siRNA, snoRNA, exRNA, scaRNA, piRNA, shRNA, Xist, HOTAIR, short non-coding RNA, long non-coding RNA, etc.)) (see, e.g., Nelles et al. (2016) “Programmable RNA Tracking in Live Cells with CRISPR/Cas9” Cell 165: 488, incorporated herein by reference). In some embodiments, the technology comprises use of an RNA-targeting protein (e.g., Cas13), which works according to a similar mechanism as Cas9. In addition to targeting genomic DNA, Cas9 and other CRISPR related proteins (e.g. Cas13) also target RNAs directed by gRNAs (see, e.g., Abudayyeh et al. (2017) “RNA targeting with CRISPR-Cas13” Nature 550: 280, incorporated herein by reference). Thus, in some embodiments, labeled gRNAs complex with dCas9 or other RNA-guided nucleases (e.g., a class 2 type VI RNA-guided RNA-targeting CRISPR-Cas effector (e.g., Cas13), a dCpf1, etc.) to visualize and track dynamics of sequence-specific RNA transcripts and non-coding RNAs in cells. Accordingly, in some embodiments, the technology relates to labeling RNAs using fluorescent guide RNAs in complex with a dCas9 or an RNA-targeting Cas13.

### Synthesis and Assembly of RNP and Delivery of RNP

In some embodiments, the protein is synthesized, purified, and assembled in vitro. In some embodiments, the gRNA is transcribed in vitro. In some embodiments, the gRNA is chemically synthesized de novo. In some embodiments, the RNP complex is assembled in vitro using in vitro-transcribed, or de novo-synthesized single guide RNA (sgRNA) and a protein that is synthesized, purified, and folded in vitro.

In some embodiments, an expression system (e.g., comprising an expression vector and a suitable expression host) finds use in producing a polypeptide and/or the RNA of the RNP. Numerous suitable expression vectors are known to those of skill in the art, and many are commercially available. The following vectors are provided by way of example; for eukaryotic host cells: pXT1, pSG5 (Stratagene), pSVK3, pBPV, pMSG, and pSVLSV40 (Pharmacia). However, any other vector may be used so long as it is compatible with the host cell. Depending on the host/vector system utilized, any of a number of suitable transcription and translation control elements, including constitutive and inducible promoters, transcription enhancer elements, transcription terminators, etc. may be used in the expression vector (see e.g., Bitter et al. (1987) Methods in Enzymology, 153:516-544, incorporated herein by reference).

In some embodiments, the protein (e.g., the Cas9 or Cas9 derivative protein) is chemically modified with a fluorescent dye, fluorescent protein, luciferase, heavy metal, magnetic probe, electric probe, ultrasound label, quantum dot, biotin, antibody, infrared label, magnetic particle, split probe (FRET), rolling circle base, or any other probe that can be detected via fluorescence, radiation, magnetism, electricity, ultrasound, or luminescence (see, e.g., FIG. 3).

In some embodiments, the protein is provided as a single polypeptide (e.g., a full Cas9 or dCas9 protein). In some embodiments, the protein is provided in multiple polypeptides, e.g., a split Cas9 or dCas9 protein provided in two parts, three parts, etc.

Similarly, embodiments provide that the gRNA is chemically modified with a fluorescent dye, fluorescent protein, luciferase, heavy metal, magnetic probe, electric probe, ultrasound label, quantum dot, biotin, antibody, infrared label, magnetic particle, split probe (FRET), rolling circle base, or any other probe that can be detected via fluorescence, radiation, magnetism, electricity, ultrasound, or luminescence.

In some embodiments, the RNP is provided as a nanoparticle for administration to a live organism for in vivo and/or in situ imaging.

In some embodiments, the RNP is delivered into cells using a technique or composition related to nucleofection, cell penetrating peptide, viral vesicles, cell surface tunneling protein, ultrasound, electroporation, cell squeezing, nanoparticles, gold or other metal particles, lipid particles, liposomes, viral transduction, viral particles, cell-cell fusion, ballistics, microinjection, and exosome intake.

In some embodiments, the protein comprises a nuclear localization signal (NLS), e.g., an SV40 NLS, to direct the RNP to enter a nucleus. In some embodiments, the protein (e.g., a Cas9, a dCas9, a GFP-dCas9) comprises an importin beta binding (IBB) domain sequence, e.g., to promote import of the polypeptide into a cell nucleus, e.g., by an importin (see, e.g., Lott and Cingolani (2011), Biochim Biophys Acta 1813(9): 1578-92, incorporated herein by reference).

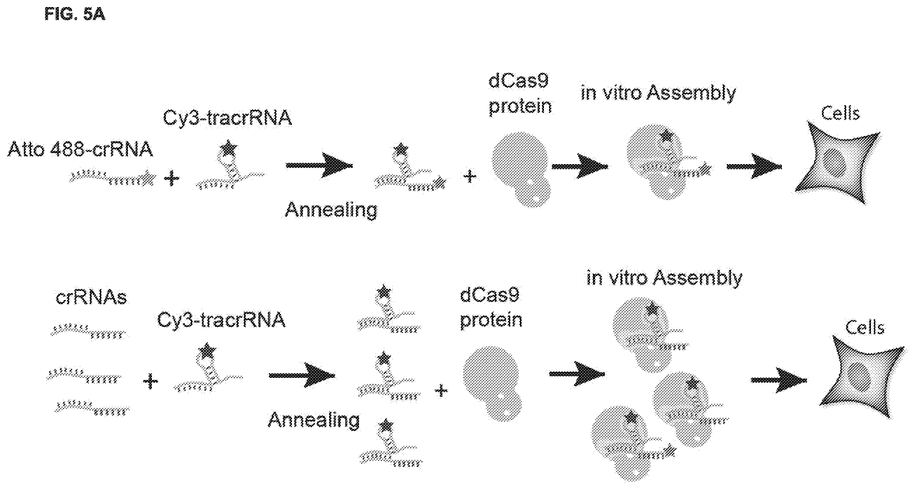

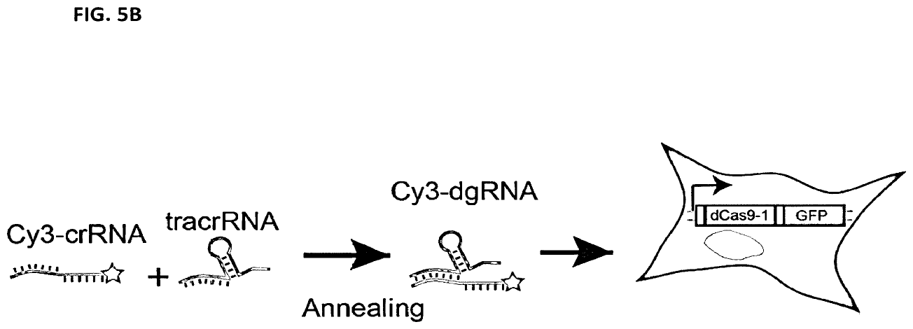

In some embodiments, an RNA is introduced into a cell that expresses a Cas9 or dCas9 polypeptide (see, e.g., FIG. 5B). In some embodiments, labeled crRNA/tracrRNA complexes (e.g., comprising a labeled crRNA and/or a labeled trarcrRNA) are introduced into cells stably expressing a Cas9 or dCas9 protein (see, e.g., FIG. 5B). In some embodiments, labeled sgRNA is introduced into cells stably expressing a Cas9 or dCas9 protein.

### Labeling Moieties

Accordingly, provided herein is a dCas9/gRNA-based approach for imaging, detecting, and/or isolating nucleic acids (e.g., double-stranded genomic DNA), e.g., using a detectable Cas9 (e.g., dCas9) protein and/or a detectable gRNA (e.g., a crRNA, a tracrRNA, a sgRNA).

In some embodiments, a gRNA and/or polypeptide of the RNP comprises a label moiety such as, e.g., a nanoparticle or a heavy metal. In some embodiments, a gRNA and/or polypeptide of the RNP comprises a phosphorescent or a luminescent label. In some embodiments, a gRNA and/or polypeptide of the RNP comprises a magnetic label. In some embodiments, a gRNA and/or polypeptide of the RNP comprises an infrared dye or a moiety that is detectable by MRI, ultrasound, SPECT, or PET technologies. In some embodiments, a gRNA and/or polypeptide of the RNP comprises a luciferase. Suitable detectors and detection functionalities are known in the art.

In some embodiments, a nucleic acid (e.g., a crRNA, a tracrRNA, a sgRNA) comprises a label. In some embodiments, a tracrRNA comprises a label, e.g., to target genomic loci. In some embodiments, a labeled tracrRNA finds use in a multiplexed imaging (see, e.g., FIG. 5A).

In some particular embodiments, a nucleic acid (e.g., a crRNA, a tracrRNA, a sgRNA) comprises a fluorescent moiety (e.g., a fluorogenic dye, also referred to as a “fluorophore” or a “fluor”). In some embodiments, a protein comprises a label. In some particular embodiments, a protein comprises a fluorescent moiety (e.g., a fluorogenic dye, also referred to as a “fluorophore” or a “fluor”). A wide variety of fluorescent moieties is known in the art and methods are known for linking a fluorescent moiety to a nucleotide prior to incorporation of the nucleotide into an oligonucleotide and for adding a fluorescent moiety to an oligonucleotide after synthesis of the oligonucleotide.

Examples of compounds that may be used as the fluorescent moiety include but are not limited to xanthene, anthracene, cyanine, porphyrin, and coumarin dyes. Examples of xanthene dyes that find use with the present technology include but are not limited to fluorescein, 6-carboxyfluorescein (6-FAM), 5-carboxyfluorescein (5-FAM), 5- or 6-carboxy-4,7,2′,7′-tetrachlorofluorescein (TET), 5- or 6-carboxy-4′5′2′4′5′7′ hexachlorofluorescein (HEX), 5′ or 6′-carboxy-4′,5′-dichloro-2,‘7’-dimethoxyfluorescein (JOE), 5-carboxy-2′,4′,5′,7′-tetrachlorofluorescein (ZOE), rhodol, rhodamine, tetramethylrhodamine (TAMRA), 4,7-dlchlorotetramethyl rhodamine (DTAMRA), rhodamine X (ROX), and Texas Red. Examples of cyanine dyes that may find use with the present invention include but are not limited to Cy 3, Cy 3B, Cy 3.5, Cy 5, Cy 5.5, Cy 7, and Cy 7.5. Other fluorescent moieties and/or dyes that find use with the present technology include but are not limited to energy transfer dyes, composite dyes, and other aromatic compounds that give fluorescent signals. In some embodiments, the fluorescent moiety comprises a quantum dot.

In some embodiments, the fluorescent moiety comprises a fluorescent protein (e.g., a green fluorescent protein (GFP), a modified derivative of GFP (e.g., a GFP comprising S65T, an enhanced GFP or “EGFP” (e.g., comprising F64L)), or others known in the art such as, e.g., blue fluorescent protein (e.g., EBFP, EBFP2, Azurite, mKalama1), cyan fluorescent protein (e.g., ECFP, Cerulean, CyPet, mTurquoise2), and yellow fluorescent protein and yellow fluorescent protein derivatives (e.g., YFP, Citrine, Venus, YPet). Embodiments provide that the fluorescent protein may be covalently or noncovalently bound to a protein or nucleic acid.

In some embodiments, the fluorescent moiety is a HaloTag comprising a protein that forms a complex with a fluorescent dye.

Fluorescent dyes include, without limitation, d-Rhodamine acceptor dyes including Cy 5, dichloro[R110], dichloro[R6G], dichloro[TAMRA], dichloro[ROX] or the like, fluorescein donor dyes including fluorescein, 6-FAM, 5-FAM, or the like; Acridine including Acridine orange, Acridine yellow, Proflavin (pH 7), or the like; Aromatic Hydrocarbons including 2-Methylbenzoxazole, Ethyl p-dimethylaminobenzoate, Pyrrole, or the like; Arylmethine Dyes including Auramine O, Crystal violet, Crystal violet, Malachite Green or the like; Coumarin dyes including 7-Methoxycoumarin-4-acetic acid, Coumarin 1, Coumarin 30, Coumarin 314, Coumarin 343, Coumarin 6 or the like; Cyanine Dyes including 1,1′-diethyl-2,2′-cyanine iodide, Cryptocyanine, Indocarbocyanine (C3) dye, Indodicarbocyanine (C5) dye, Indotricarbocyanine (C7) dye, Oxacarbocyanine (C3) dye, Oxadicarbocyanine (C5) dye, Oxatricarbocyanine (C7) dye, Pinacyanol iodide, Stains all, Thiacarbocyanine (C3) dye, Thiadicarbocyanine (C5) dye, Thiatricarbocyanine (C7) dye, or the like; Dipyrrin dyes including N,N′-Difluoroboryl-1,9-dimethyl-5-(4-iodophenyl)-dipyrrin, N,N′-Difluoroboryl-1,9-dimethyl-5-[(4-(2-trimethylsilylethynyl), N,N′-Difluoroboryl-1,9-dimethyl-5-phenydipyrrin, or the like; Merocyanines including 4-(dicyanomethylene)-2-methyl-6-(p-dimethylaminostyryl)-4H-pyran (DCM), 4-Dimethylamino-4′-nitrostilbene, Merocyanine 540, or the like; Miscellaneous Dyes including 4′,6-Diamidino-2-phenylindole (DAPI), dimethylsulfoxide, 7-Benzylamino-4-nitrobenz-2-oxa-1,3-diazole, Dansyl glycine, dioxane, Hoechst 33258, Lucifer yellow CH, Piroxicam, Quinine sulfate, Squarylium dye III, or the like; Oligophenylenes including 2,5-Diphenyloxazole (PPO), Biphenyl, POPOP, p-Quaterphenyl, p-Terphenyl, or the like; Oxazins including Cresyl violet perchlorate, Nile Blue, Nile Red, Oxazine 1, Oxazine 170, or the like; Polycyclic Aromatic Hydrocarbons including 9,10-Bis(phenylethynyl)anthracene, 9,10-Diphenylanthracene, Anthracene, Naphthalene, Perylene, Pyrene, or the like; polyene/polyynes including 1,2-diphenylacetylene, 1,4-diphenylbutadiene, 1,4-diphenylbutadiyne, 1, 6-Diphenylhexatriene, Beta-carotene, Stilbene, or the like; Redox-active Chromophores including Anthraquinone, Azobenzene, Benzoquinone, Ferrocene, Riboflavin, Tris(2,2′-bipyridypruthenium(II), Tetrapyrrole, Bilirubin, Chlorophyll a, Chlorophyll b, Diprotonated-tetraphenylporphyrin, Hematin, Magnesium octaethylporphyrin, Magnesium octaethylporphyrin (MgOEP), Magnesium phthalocyanine (MgPc), Magnesium tetramesitylporphyrin (MgTMP), Magnesium tetraphenylporphyrin (MgTPP), Octaethylporphyrin, Phthalocyanine (Pc), Porphin, ROX, TAMRA, Tetra-t-butylazaporphine, Tetra-t-butylnaphthalocyanine, Tetrakis(2,6-dichlorophenyl)porphyrin, Tetrakis(o-aminophenyl)porphyrin, Tetramesitylporphyrin (TMP), Tetraphenylporphyrin (TPP), Vitamin B12, Zinc octaethylporphyrin (ZnOEP), Zinc phthalocyanine (ZnPc), Zinc tetramesitylporphyrin (ZnTMP), Zinc tetramesitylporphyrin radical cation, Zinc tetraphenylporphyrin (ZnTPP), or the like; Xanthenes including Eosin Y, Fluorescein, Rhodamine 123, Rhodamine 6G, Rhodamine B, Rose bengal, Sulforhodamine 101, or the like; or mixtures or combination thereof or synthetic derivatives thereof.

Several classes of fluorogenic dyes and specific compounds are known that are appropriate for particular embodiments of the technology: xanthene derivatives such as fluorescein, rhodamine, Oregon green, eosin, and Texas red; cyanine derivatives such as cyanine, indocarbocyanine, oxacarbocyanine, thiacarbocyanine, and merocyanine; naphthalene derivatives (dansyl and prodan derivatives); coumarin derivatives; oxadiazole derivatives such as pyridyloxazole, nitrobenzoxadiazole, and benzoxadiazole; pyrene derivatives such as cascade blue; oxazine derivatives such as Nile red, Nile blue, cresyl violet, and oxazine 170; acridine derivatives such as proflavin, acridine orange, and acridine yellow; arylmethine derivatives such as auramine, crystal violet, and malachite green; and tetrapyrrole derivatives such as porphin, phtalocyanine, bilirubin.

In some embodiments the fluorescent moiety a dye that is xanthene, fluorescein, rhodamine, BODIPY, cyanine, coumarin, pyrene, phthalocyanine, phycobiliprotein.

In some embodiments, the fluorescent moiety is, e.g., ALEXA FLUOR® 350, ALEXA FLUOR® 405, ALEXA FLUOR® 430, ALEXA FLUOR® 488, ALEXA FLUOR® 514, ALEXA FLUOR® 532, ALEXA FLUOR® 546, ALEXA FLUOR® 555, ALEXA FLUOR® 568, ALEXA FLUOR® 568, ALEXA FLUOR® 594, ALEXA FLUOR® 610, ALEXA FLUOR® 633, ALEXA FLUOR® 647, ALEXA FLUOR® 660, ALEXA FLUOR® 680, ALEXA FLUOR® 700, ALEXA FLUOR® 750, or a squaraine dye. In some embodiments, the label is a fluorescently detectable moiety as described in, e.g., Haugland (September 2005) MOLECULAR PROBES HANDBOOK OF FLUORESCENT PROBES AND RESEARCH CHEMICALS (10th ed.), which is herein incorporated by reference.

In some embodiments, the label (e.g., a fluorescently detectable label) is one available from ATTO-TEC GmbH (Am Eichenhang 50, 57076 Siegen, Germany), e.g., as described in U.S. Pat. Appl. Pub. Nos. 20110223677, 20110190486, 20110172420, 20060179585, and 20030003486; and in U.S. Pat. No. 7,935,822, each of which is incorporated herein by reference (e.g., ATTO 390, ATTO 425, ATTO 465, ATTO 488, ATTO 495, ATTO 514, ATTO 520, ATTO 532, ATTO Rho6G, ATTO 542, ATTO 550, ATTO 565, ATTO Rho3B, ATTO Rho11, ATTO Rho12, ATTO Thio12, ATTO Rho101, ATTO 590, ATTO 594, ATTO Rho13, ATTO 610, ATTO 620, ATTO Rho14, ATTO 633, ATTO 647, ATTO 647N, ATTO 655, ATTO Oxa12, ATTO 665, ATTO 680, ATTO 700, ATTO 725, ATT0740).

One of ordinary skill in the art will recognize that dyes having emission maxima outside these ranges may be used as well. In some cases, dyes ranging between 500 nm to 700 nm have the advantage of being in the visible spectrum and can be detected using existing photomultiplier tubes. In some embodiments, the broad range of available dyes allows selection of dye sets that have emission wavelengths that are spread across the detection range. Detection systems capable of distinguishing many dyes are known in the art.

In some embodiments, the imaging moiety is non-fluorescent, e.g., a chemical moiety that is not fluorescent but that is used to provide a contrast or signal in imaging and is detectable by a non-fluorescent imaging technique.

In some embodiments, imaging moieties are chemically linked to a protein or RNA as described herein. Non-limiting examples of imaging moieties include, e.g., photoluminescent nanoparticles, radioisotopes, superparamagnetic agents, X-ray contrast agents, and ultrasound agents. In some embodiments, an imaging moiety comprises therapeutic reporters such as porphyrins, Photofrin®, Lutrin®, Antrin®, aminolevulinic acid, hypericin, benzoporphryrin derivatives used in photodynamic therapy, and radionuclides used for radiotherapy.

In some embodiments, the imaging moiety is radioactive. For example, in some embodiments, the imaging moiety comprises, e.g., one or more radioactive labels, e.g., radioisotopic forms of metals such as copper, gallium, indium, technetium, yttrium, and lutetium. In some embodiments, the radioisotopic metal is chemically linked to the imaging moiety and finds use for nuclear imaging or therapeutic applications. Exemplary radioactive labels include, without limitation, ⁹⁹mTc, ¹¹In, ⁶⁴Cu, ⁶⁷Ga, ¹⁸⁶Re, ¹⁸⁸Re, ¹⁵⁸Sm, ¹⁷⁷Lu, and ⁶⁷Cu.

In some embodiments, a label comprises, e.g., ¹²³I, ¹²⁴I, ¹²⁵I, ¹¹C, ¹³N, ¹⁵O, or ¹⁸F. Other exemplary labels comprise, for example, ¹⁸⁶Re, ¹⁸⁸Re, ¹⁵³Sm, ¹⁶⁶Ho, ¹⁷⁷Lu, ¹⁴⁹Pm, ⁹⁰Y, ²¹²Bi, ¹⁰³Pd, ¹⁰⁹Pd, ¹⁵⁹Gd, ¹⁴⁰La, ¹⁹⁸Au, ¹⁹⁹Au, ¹⁶⁹Yb, ¹⁷⁵Yb, ¹⁶⁵Dy, ¹⁶⁶Dy, ⁶⁷Cu, ¹⁰⁵Rh, ¹¹¹Ag, and ¹⁹²Ir. In some embodiments, the label finds use as a therapeutic radiopharmaceutical.

In some embodiments, the imaging moiety comprises a chelator or bonding moiety. In some embodiments, chelators are selected to form stable complexes with radioisotopes that have imageable gamma ray or positron emissions, such as ^{99m}Tc, ¹¹¹In, ⁶⁴Cu, and ⁶⁷Ga. Exemplary chelators include diaminedithiols, monoamine-monoamidedithiols, triamide-monothiols, monoamine-diamide-monothiols, diaminedioximes, and hydrazines. Chelators generally are tetradentate with donor atoms selected from nitrogen, oxygen and sulfur, and may include for example, cyclic and acyclic polyaminocarboxylates such as diethylenetriaminepentaacetic acid (DTPA), 1,4,7,10-tetraazacyclododecane-1,4,7,10-tetraacetic acid (DOTA), (DO3A), 2-benzyl-DOTA, alpha-(2-phenethyl)1,4,7,10-tetraazazcyclododecane-1-acetic-4,7,10-tris(m-ethylacetic)acid, 2-benzyl-cyclohexyldiethylenetriaminepentaacetic acid, 2-benzyl-6-methyl-DTPA, and 6,6″-bis[N,N,N″,N″-tetra(carboxymethyl)aminomethyl)-4′-(3-amino-4-meth-oxyphenyl)-2,2′:6′,2″-terpyridine.

In some embodiments, the imaging moiety comprises a magnetic label. In some embodiments, an imaging moiety comprises a chelating agent for a magnetic resonance imaging agent, e.g., polyamine-polycarboxylate chelators or iminoacetic acid chelators that can be chemically linked to a polypeptide or RNA of an RNP as described herein. Exemplary chelators for magnetic resonance imaging agents are selected to form stable complexes with paramagnetic metal ions, such as Gd(III), Dy(III), Fe(III), and Mn(II). For example, some exemplary chelators are, e.g., cyclic and acyclic polyaminocarboxylates such as DTPA, DOTA, DO3A, 2-benzyl-DOTA, alpha-(2-phenethyl)1,4,7,10-tetraazacyclododecane-1-acetic-4,7,10-tris(me-thylacetic)acid, 2-benzyl-cyclohexyldiethylenetriaminepentaacetic acid, 2-benzyl-6-methyl-DTPA, and 6,6″-bis[N,N,N″,N″-tetra(carboxymethyeaminomethyl)-4′-(3-amino-4-metho-xyphenyl)-2,2′:6′,2″-terpyridine.

In some embodiments, an imaging moiety comprises a superparamagnetic metal oxide nanoparticle, e.g., that is either non-fluorescent or fluorescent and can be used in a variety of in vitro and in vivo applications. Fluorescent metal oxide nanoparticles that also have magnetic properties can be used for MRI, thus providing a multi-modality imaging agent.

In certain embodiments, the imaging moiety comprises a fluorescent and/or non-fluorescent superparamagenetic metal oxide nanoparticle. In some embodiments, the imaging moiety comprises a polymer coating suitable for attaching a plurality of agents.

In some embodiments, the imaging moiety is an ultrasound label. For ultrasound, the imaging moiety comprises, in some embodiments, particles or metal chelates where the metal ions have atomic numbers 21-29, 42, 44 or 57-83. Examples of such compounds are described in Tyler et al., ULTRASONIC IMAGING, 3, pp. 323-29 (1981) and Swanson, “Enhancement Agents for Ultrasound: Fundamentals,” PHARMACEUTICALS IN MEDICAL IMAGING, pp. 682-87 (1990), incorporated herein by reference.

In some embodiments, the imaging moiety comprises an X-Ray label. Exemplary reporters comprise iodinated organic molecules or chelates of heavy metal ions of atomic numbers 57 to 83. Examples of such compounds are described in Sovak, ed., “Radiocontrast Agents,” SPRINGER-VERLAG, pp. 23-125 (1984) and U.S. Pat. No. 4,647,447, incorporated herein by reference.

### Isolation of Nucleic Acid

In some embodiments, a nucleic acid (e.g., a gRNA, e.g., a crRNA, a tracrRNA, a sgRNA) comprises an attachment chemistry label or linker (e.g., biotin, amino modifier) for binding to other labeling reagents. In some embodiments, a nucleic acid (e.g., a crRNA, a tracrRNA, a sgRNA) comprises an attachment chemistry label or linker (e.g., biotin, amino modifier) for binding a nucleic acid or complexes comprising a nucleic acid to be isolated, e.g., from a cell. For example, in some embodiments, an RNA (e.g., a gRNA, e.g., a crRNA, a tracrRNA, a sgRNA) is labeled with biotin and strepatavidin beads are used to isolate a nucleic acid bound to the RNP by its binding to the gRNA. The technology finds use for studying epigenetic modifications or binding proteins of a given targeted sequence.

### Isolation, Detection, and Characterization of Locus-Specific Chromatin Complex/Interactions Using Affinity Tagged Guide RNA

In some embodiments, the technology relates to in situ capture of chromatin interaction by use of affinity tagged (e.g., biotinalylated) guide RNA, e.g., as described above. See, e.g., Example 11. The technology provides an improvement over conventional technologies that use biotinylated proteins such as dCas9 (see, e.g., Liu et al. (2017) “In Situ Capture of Chromatin Interactions by Biotinylated dCas9” Cell 170: 1028-43, incorporated herein by reference). In contrast to the previous technologies (e.g., comprising use of biotinylated dCas9), in situ capture of chromatin interaction by use of affinity tagged (e.g., biotinylated) guide RNA has improved specificity and is simpler to use.

In particular, embodiments comprise isolation of locus-specific chromatin complexes, e.g., by isolating complexes comprising DNA, protein, and RNA associated with the targeted locus (e.g., using an affinity tagged (e.g., biotinylated) guide RNA, e.g., as described above). In some embodiments, the technology finds use to detect protein components associated with a specific genomic locus, such as transcriptional factors and trans-regulatory factors. In some embodiments, the technology finds use for detecting RNAs associated with a specific genomic locus. In some embodiments, the technology finds use for detecting long-range interactions between DNA elements, such as looping and topological associated domains (TADs).

In some embodiments, a nucleic acid (e.g., a gRNA, e.g., a crRNA, a tracrRNA, a sgRNA) comprises an attachment chemistry label or linker (e.g., biotin, amino modifier) for binding to affinity purification moieties. In some embodiments, a nucleic acid (e.g., a crRNA, a tracrRNA, a sgRNA) comprises an attachment chemistry label or linker (e.g., biotin, amino modifier) for binding a nucleic acid or complexes comprising a nucleic acid to be isolated, e.g., from a cell. For example, in some embodiments, an RNA (e.g., a gRNA, e.g., a crRNA, a tracrRNA, a sgRNA) is labeled with biotin and strepatavidin beads are used to isolate a nucleic acid and chromatin complexes bound to the RNP.

In some embodies, the nucleic acid (e.g., a crRNA, a tracrRNA, a sgRNA) comprises a click chemistry moiety appropriate for using in a click chemistry reaction with a second component comprising a click chemistry moiety to purify target-specific chromatin complexes and/or to identify target-specific chromatin interactions. Click chemistry moieties are discussed, e.g., in Kolb et al. (2001) Angew. Chim. Int. Ed. 40: 2004, incorporated herein by reference. For example, an alkyne reacts with an azide group (e.g., N3, e.g., N═N═N) in a copper (I)-catalyzed azide-alkyne cycloaddition (“CuAAC”) reaction to form two new covalent bonds between azide nitrogens and alkyl carbons. The covalent bonds form a chemical link (e.g., comprising a five-membered triazole ring) between a first component and a second component that comprised the azide and the alkyne moieties before linkage. This type of cycloaddition reaction is one of the foundational reactions of “click chemistry” because it provides a desirable chemical yield, is physiologically stable, and exhibits a large thermodynamic driving force that favors a “spring-loaded” reaction that yields a single product (e.g., a 1,4-regioisomer of 1,2,3-triazole). See, e.g., Huisgen (1961) “Centenary Lecture—1,3-Dipolar Cycloadditions”, Proceedings of the Chemical Society of London 357, incorporated herein by reference.

In some embodies, a nucleic acid (e.g., a crRNA, a tracrRNA, a sgRNA) comprises a modification that is recognized by a binding partner. In some embodiments a nucleic acid (e.g., a crRNA, a tracrRNA, a sgRNA) comprises an antigen and/or epitope specifically recognized by an antibody (e.g., a digoxigennin modification recognized by anti-digoxigenin antibodies) to purify target-specific chromatin interactions.

In some embodiments, the isolation/detection moiety comprises an attachment chemistry label or linker (e.g., biotin, amino modifier) for binding to affinity purification moieties. In some embodiments, the isolation/detection moiety comprises a chelator or bonding moiety. In some embodiments, the isolation/detection moiety comprises a magnetic label. In some embodiments, an isolation/detection moiety comprises a chelating agent for affinity purification agent, e.g., polyamine-polycarboxylate chelators or iminoacetic acid chelators that can be chemically linked to a polypeptide or RNA of an RNP as described herein. In some embodiments, the isolation/detection moiety comprises a click chemistry linker that reacts in a click chemistry reaction to purify target-specific chromatin complexes and/or to identify target-specific chromatin interactions. In some embodiments, the technology finds use for studying epigenetic modifications or cis-regulator elements of a given targeted sequence.

The technology is not limited in the interaction pairs that find use in isolating, detecting, and characterizing locus-specific chromatin complex/interactions using affinity tagged guide RNA. Embodiments provide that the guide RNA is tagged with (e.g., linked to) one member of an interacting pair. Embodiments provide that the RNA is covalently linked to a first member of an interacting pair and embodiments provide that the RNA is non-covalently linked to a first member of an interacting pair. In some embodiments, the two members of an interaction pair are “specific for” each other, e.g., the two members of the interaction pair bind with a K_{d} of approximately 10⁻⁹ to10⁻¹² M or stronger (e.g., having a K_{d} less than 10⁻¹² M).

For example, exemplary interacting pairs that find use in embodiments of the technology are derived from natural ligand-receptor pairs (see, e.g., Dueber et al. Synthetic protein scaffolds provide modular control over metabolic flux. Nature biotechnology 2009, 27, 753-9; Bayer et al. From cellulosomes to cellulosomics. Chem Rec 2008, 8, 364-77; Fontes et al. Cellulosomes: highly efficient nanomachines designed to deconstruct plant cell wall complex carbohydrates. Annual review of biochemistry 2010, 79, 655-81; and Morais eta 1. Cellulase-xylanase synergy in designer cellulosomes for enhanced degradation of a complex cellulosic substrate. mBio 2010, 1, each of which is incorporated herein by reference), e.g., scFv antibodies, signaling receptors, and cohesins (e.g., a cohesin-dockerin interacting pair; see, e.g., Bayer et al. (2004) “The cellulosomes: multienzyme machines for degradation of plant cell wall polysaccharides” Annu Rev Microbiol. 58: 521-54; Zverlov et al. (2008) “Mutations in the Scaffoldin Gene, cipA, of Clostridium thermocellum with Impaired Cellulosome Formation and Cellulose Hydrolysis: Insertions of a New Transposable Element, IS1447, and Implications for Cellulase Synergism on Crystalline Cellulose” J Bacteriol. 190(12): 4321-4327, each of which is incorporated herein by reference).

In some embodiments, the interacting pair comprises, e.g., a cohesin (e.g., a cohesin module or cohesin domain) and a dockerin (e.g., a dockerin module or dockerin domain), an SH3 ligand and an SH3 domain, a PDZ ligand and a PDZ domain, an antibody and epitope (antigen), an aptamer and aptamer ligand, etc.

### Methods

In some embodiments, the technology provided herein relates to methods for imaging (e.g., detecting) a target DNA, e.g., in a cell (e.g., a living cell, e.g., a living primary cell). Generally, a method involves contacting a target DNA with a RNP complex (a “targeting complex”), which complex comprises a DNA-targeting RNA (e.g., comprising a detectable label) and a Cas9 (e.g., dCas9) polypeptide.

As discussed herein, a DNA-targeting RNA and a polypeptide (e.g., a Cas9 or dCas9) form a ribonucleoprotein (RNP) complex. The DNA-targeting RNA provides target specificity to the RNP complex by comprising a nucleotide sequence that is complementary to a sequence of a target DNA. The polypeptide (e.g., a Cas9 or dCas9) of the RNP complex provides the site-specific activity. In some embodiments, a RNP complex images (e.g., identifies, detects) a target DNA. The target DNA may be, for example, naked DNA in vitro, chromosomal DNA in cells in vitro, chromosomal DNA in cells in vivo, etc.

In some cases, the RNP complex images target DNA at a target DNA sequence defined by the region of complementarity between the DNA-targeting RNA and the target DNA. In some cases, when the polypeptide is a Cas9 or Cas9 related polypeptide, site-specific imaging of the target DNA occurs at locations determined by both (i) base-pairing complementarity between the DNA targeting RNA and the target DNA; and (ii) a short motif (referred to as the protospacer adjacent motif (PAM)) in the target DNA. In some embodiments (e.g., when Cas9 from S. pyogenes, or a closely related Cas9, is use), the PAM sequence of the non-complementary strand is 5′-XGG-3′, where X is any DNA nucleotide and X is immediately 3′ of the target sequence of the non-complementary strand of the target DNA. As such, the PAM sequence of the complementary strand is 5′-CCY-3′, where Y is any DNA nucleotide and Y is immediately 5′ of the target sequence of the complementary strand of the target DNA. In some such embodiments, X and Y can be complementary and the X-Y base pair can be any base pair (e.g., X═C and Y=G; X=G and Y═C; X=A and Y=T, X=T and Y=A). In some embodiments, the RNP has no requirement for a PAM sequence.

In some embodiments, methods comprise a step of producing a polypeptide (e.g., a Cas9, dCas9, and/or a modified variant thereof) in vitro. In some embodiments, methods comprise a step of producing a nucleic acid in vitro, e.g., an RNA, e.g., one or more of a tracrRNA, a crRNA, and/or a sgRNA. In some embodiments, methods comprise a step of folding and/or assembling RNA (e.g., folding and/or annealing a tracrRNA and a crRNA; folding a sgRNA, folding and/or annealing a dgRNA). In some embodiments, methods comprise a step of assembling a RNP complex in vitro, e.g., a RNP comprising a polypeptide and one or more RNA molecules. In some embodiments, methods comprise a step of introducing a RNP into a cell (e.g., a living cell, e.g., a living primary cell).

In some embodiments, multiple DNA-targeting RNAs and/or multiple RNPs are used simultaneously to simultaneously image (e.g., identify, detect) different nucleic acid sequences on the same target DNA or on different target DNAs, e.g., to provide a multiplex method. In some embodiments, two or more DNA-targeting RNAs target the same gene or transcript or locus. In some embodiments, two or more DNA-targeting RNAs target different unrelated loci. In some embodiments, two or more DNA-targeting RNAs target different, but related loci.

In some embodiments, the polypeptide (e.g., a Cas9 or dCas9) is provided directly as a protein. In some embodiments, a nucleic acid is introduced into a cell and the polypeptide (e.g., a Cas9 or dCas9) is expressed from the nucleic acid in the cell. As one non-limiting example, fungi (e.g., yeast) can be transformed with exogenous protein, nucleic acid, and/or RNP complexes using spheroplast transformation (see Kawai et al., Bioeng Bugs. 2010 November-December; 1(6):395-403: “Transformation of Saccharomyces cerevisiae and other fungi: methods and possible underlying mechanism”; and Tanka et al., Nature. 2004 Mar. 18; 428(6980323-8: “Conformational variations in an infectious protein determine prion strain differences”; each of which is herein incorporated by reference). Thus, a polypeptide (e.g., dCas9), nucleic acid (e.g., RNA), and/or a RNP can be incorporated into a spheroplast and the spheroplast can be used to introduce the RNP into a yeast cell. A RNP can be introduced into a cell (provided to the cell) by any convenient method; such methods are known to those of ordinary skill in the art. As another non-limiting example, a RNP can be injected directly into a cell, e.g., a human cell, a cell of a zebrafish embryo, the pronucleus of a fertilized mouse oocyte, etc.

### Cells

In some embodiments of the technology provided herein, the technology finds use to image (e.g., detect, identify) nucleic acids in mitotic or post-mitotic cells in vivo and/or ex vivo and/or in vitro. Because the DNA-targeting RNA provides specificity by hybridizing to target DNA, a mitotic and/or post-mitotic cell of interest in the disclosed methods may include a cell from any organism (e.g., a bacterial cell, an archaeal cell, a cell of a single-cell eukaryotic organism, a plant cell, an algal cell, e.g., Botryococcus braunii, Chlamydomonas reinhardtii, Nannochloropsis gaditana, Chlorella pyrenoidosa, Sargassum patens C. Agardh, and the like, a fungal cell (e.g., a yeast cell), an animal cell, a cell from an invertebrate animal (e.g. fruit fly, cnidarian, echinoderm, nematode, etc.), a cell from a vertebrate animal (e.g., fish, amphibian, reptile, bird, mammal), a cell from a mammal, a cell from a rodent, a cell from a human, etc.).

Any type of cell may be of interest (e.g. a stem cell, e.g. an embryonic stem (ES) cell, an induced pluripotent stem (iPS) cell, a germ cell; a somatic cell, e.g. a fibroblast, a hematopoietic cell, a neuron, a muscle cell, a bone cell, a hepatocyte, a pancreatic cell; an in vitro or in vivo embryonic cell of an embryo at any stage, e.g., a 1-cell, 2-cell, 4-cell, 8-cell, etc. stage zebrafish embryo; etc.). Cells may be from established cell lines or they may be primary cells, where “primary cells”, “primary cell lines”, and “primary cultures” are used interchangeably herein to refer to cells and cells cultures that have been derived from a subject and allowed to grow in vitro for a limited number of passages (e.g, “splittings”) of the culture. For example, primary cultures are cultures that may have been passaged 0 times, 1 time, 2 times, 4 times, 5 times, 10 times, or 15 times, but not enough times go through the crisis stage. Typically, the primary cell lines of the present invention are maintained for fewer than 10 passages in vitro. Target cells are in many embodiments unicellular organisms or are grown in culture.

In some embodiments, primary cells are obtained from an individual by any convenient method. For example, leukocytes may be conveniently obtained by apheresis, leukocytapheresis, density gradient separation, etc., while cells from tissues such as skin, muscle, bone marrow, spleen, liver, pancreas, lung, intestine, stomach, etc. are most conveniently obtained by biopsy. An appropriate solution may be used for dispersion or suspension of the obtained cells. Such solution will generally be a balanced salt solution, e.g. normal saline, phosphate-buffered saline (PBS), Hank's balanced salt solution, etc., conveniently supplemented with fetal calf serum or other naturally occurring factors, in conjunction with an acceptable buffer at low concentration, generally from 5-25 mM. Convenient buffers include HEPES, phosphate buffers, lactate buffers, etc. The cells may be used immediately, or they may be stored, frozen, for long periods of time, being thawed and capable of being reused. In such cases, the cells will usually be frozen in 10% DMSO, 50% serum, 40% buffered medium, or some other such solution as is commonly used in the art to preserve cells at such freezing temperatures, and thawed in a manner as commonly known in the art for thawing frozen cultured cells.

### Multiplex

Some embodiments comprise use of a plurality of labels to image a plurality of nucleic acids, e.g., some embodiments comprise use of a plurality of distinguishable labels (e.g., a first label, a second label, etc.) to image a plurality (e.g., different) nucleic acids (e.g., a first nucleic acid, a second nucleic acid, etc.). In some embodiments, the plurality of nucleic acids is in the same cell, sub-cellular location, or organelle, etc. Some embodiments comprise use of a plurality of labels to image a plurality of nucleic acids in parallel, e.g., simultaneously.

For instance, the technology provides a multiplex method to perform parallel detection of a plurality of biomarkers associated with a normal or non-normal biological state of a subject. For example, in some embodiments the technology provides a multiplex method to perform parallel detection of a plurality of disease markers. In some embodiments, parallel detection of a plurality of biomarkers comprises use of multiple gRNAs (e.g., comprising distinguishable labels) to image different the different biomarkers, e.g., that are indicative of a disease or a non-normal state.

Thus, in some embodiments, the RNP and methods for RNP delivery and imaging comprise use of labeled gRNAs to provide multiplexed imaging of multiple genomic loci in living cells. In exemplary embodiments, the platform finds use for diagnosis of nucleic acid variations associated with disease, e.g., detecting a plurality of gene mutations (substitutions, insertions, deletions), e.g., in living cells. In some embodiments, the technology finds use in visualizing genomic dynamics in living cells (e.g., primary cells) and tracking the dynamic movements (e.g., changes in location (x, y, z) of nucleic acids with respect to time (t)) of multiple genomic loci in living cells (e.g., primary cells).

### Time-Resolved Dynamic Imaging

In some embodiments, the technology described herein provides a time-resolved imaging of nucleic acids (e.g., chromosomes, genetic loci, genomes, genes) in live cells. In particular, in some embodiments the technology provides methods for recording the location of a nucleic acid in one, two, or three-dimensional space (e.g., using one, two, or three spatial coordinates) and recording a time (e.g., using a temporal coordinate) associated with each recorded location, e.g., to provide a set of coordinates comprising spatial and temporal coordinates (e.g., x, y, z, t). In some embodiments, a vector is computed describing the movement of a nucleic acid in space with respect to time. In some embodiments, a plurality of vectors is calculated describing the movement of, and changes in the movement of (e.g., accelerations of), a nucleic acid in space with respect to time. In some embodiments, kinetic calculations are performed using the recorded spatial and temporal coordinates.

In some embodiments, recording a spatial and time coordinates comprises recording a movie or series of time-stamped still images.

In some embodiments, detecting a variation in a nucleic acid (e.g., a difference in a nucleic acid with respect to a normal or wild-type nucleic acid) comprises detecting a difference in the change of one or more spatial coordinates (e.g., x1, y1, z1) with respect to time (t1) relative to the change of one or more spatial coordinates (e.g., x2, y2, z2) with respect to time (t2) for a known control or wild-type sample. For example, detecting a movement of a chromosome that is different than a movement of a normal chromosome may indicate that the chromosome is aneuploid or otherwise is not normal.

In some embodiments, recording spatial and time coordinates for a nucleic acid comprises measuring the location of the nucleic acid on a time scale ranging from (e.g., recording a movie of the nucleic acid for a time ranging from) approximately 0.1 millisecond to approximately 10 days (e.g., approximately 1, 2, 3, 4, 5, 6, 7, 8, or 9×10⁻⁴ seconds; approximately 1, 2, 3, 4, 5, 6, 7, 8, or 9×10⁻³ seconds; approximately 1, 2, 3, 4, 5, 6, 7, 8, or 9×10⁻² seconds; approximately 1, 2, 3, 4, 5, 6, 7, 8, or 9×10⁻¹ seconds; approximately 1, 2, 3, 4, 5, 6, 7, 8, or 9 seconds; approximately 1, 2, 3, 4, 5, 6, 7, 8, or 9×10 seconds; approximately 1, 2, 3, 4, 5, 6, 7, 8, or 9×10² seconds; approximately 1, 2, 3, 4, 5, 6, 7, 8, or 9×10³ seconds; approximately 1, 2, 3, 4, 5, 6, 7, 8, or 9×10⁴ seconds; approximately 1, 2, 3, 4, 5, 6, 7, 8, or 9×10⁵ seconds; or approximately 1, 2, 3, 4, 5, 6, 7, 8, or 9×10⁶ seconds).

### Samples

In some embodiments, nucleic acids (e.g., DNA or RNA, e.g., chromosomes, genes, genetic loci, genetic markers, etc.) are imaged in a biological sample containing a variety of other components, such as proteins, lipids, and non-target nucleic acids. In some embodiments, samples are obtained from and/or comprise and/or are derived or prepared from a variety of materials (e.g., cellular material (live or dead), extracellular material, viral material, environmental samples (e.g., metagenomic samples), synthetic material (e.g., amplicons such as provided by PCR or other amplification technologies)), obtained from an animal, plant, bacterium, archaeon, fungus, or any other organism. Biological samples for use in the present technology include viral particles or preparations thereof. In some embodiments, sample are obtained directly from an organism or from a biological sample obtained from an organism, e.g., blood, urine, cerebrospinal fluid, seminal fluid, saliva, sputum, stool, hair, sweat, tears, skin, amniotic fluid, and tissue (e.g., umbilical tissue). Exemplary samples include, but are not limited to, whole blood, lymphatic fluid, serum, plasma, buccal cells, sweat, tears, saliva, sputum, hair, skin, biopsy, cerebrospinal fluid (CSF), amniotic fluid, seminal fluid, vaginal excretions, serous fluid, synovial fluid, pericardial fluid, peritoneal fluid, pleural fluid, transudates, exudates, cystic fluid, bile, urine, gastric fluids, intestinal fluids, fecal samples, and swabs, aspirates (e.g., bone marrow, fine needle, etc.), washes (e.g., oral, nasopharyngeal, bronchial, bronchialalveolar, optic, rectal, intestinal, vaginal, epidermal, etc.), and/or other specimens.

Any tissue or body fluid specimen may be used as a sample or a source of a sample for use in the technology, including forensic specimens, archived specimens, preserved specimens, and/or specimens stored for long periods of time, e.g., fresh-frozen, methanol/acetic acid fixed, or formalin-fixed paraffin embedded (FFPE) specimens and samples. In particular embodiments, the sample comprises cultured cells, such as a primary cell culture or a cell line. In some specific embodiments, the sample comprises live primary cells.

In some embodiments, sample (e.g., the cells or tissues) are infected with a virus or other intracellular pathogen. A sample can also be isolated from a non-cellular origin, e.g. amplified/isolated nucleic acid that has been stored in a freezer.

In some embodiments of the technology, the technology is applied in vivo, ex vivo, and/or in vitro. In some embodiments, the technology is used to image a sample in situ, e.g., without removing it from a subject or a patient. In some embodiments, the sample is a crude sample, a minimally treated cell lysate, or a biofluid lysate.

### Subjects and Diseases

In some embodiments, the technology finds use in the imaging of a sample from a subject. In some embodiments, the technology finds use in diagnosing a subject. In some embodiments, the technology finds use in detecting an aneuploidy in a sample from a subject. Examples of aneuploidies include, but are not limited to, monosomies, aberrant disomies (e.g., when a number other than two chromosomes is normal), trisomies, or higher polysomies and multisomies. In some embodiments, a subject has an autosomal aneuploidy and in some embodiments a subject has a sex chromosome aneuploidy. In some embodiments, the aneuploidy is of human chromosome 1, 2, 3, 4, 5, 6, 7, 8, 9, 10, 11, 12, 13, 14, 15, 16, 17, 18, 19, 20, 21, and/or 22; in some embodiments the aneuploidy is of human chromosome X and/or Y. In some embodiments, the aneuploidy is monosomy of human chromosome 1, 2, 3, 4, 5, 6, 7, 8, 9, 10, 11, 12, 13, 14, 15, 16, 17, 18, 19, 20, 21, and/or 22; in some embodiments the aneuploidy is monosomy of human chromosome X and/or Y. In some embodiments, the aneuploidy is trisomy of human chromosome 1, 2, 3, 4, 5, 6, 7, 8, 9, 10, 11, 12, 13 (e.g., Patau Syndrome), 14, 15, 16, 17, 18, 19, 20, 21 (Down Syndrome), and/or 22; in some embodiments the aneuploidy is trisomy of human chromosome X and/or Y.

In some embodiments, the technology finds use in diagnosing a subject having an aberrant copy number of a gene (e.g., too few or too many functional copies of a gene or gene function). In some embodiments, the technology finds use in diagnosing a subject having abnormal expression of a gene (e.g., two few or too many mRNAs expressed (e.g., transcribed) from a gene). In some embodiments, the technology finds use in detecting a gene fusion. In some embodiments, the technology finds use in detecting a deletion of a gene. In some embodiments, the technology finds use in detecting a chromosome translocation. In some embodiments, the technology finds use in detecting the amplification of a gene. In some embodiments, the technology finds use in detecting sequence variation in a gene (e.g., an insertion, deletion, polymorphism (e.g., SNP)).

Accordingly, the technology finds use in imaging samples from a subject having a genetic disease; a cancer; a blood disease; an autoimmune disease; a neurodegenerative disease (e.g., Huntinton disease; amyotrophic laterals sclerosis; Parkinson disease; Alzheimer disease); disease due to repetitive sequence expansion in a chromosome; disease due to microsatellite DNA (e.g., Lynch syndrome); disease due to chromosome rearrangement; disease due to chromosome deletion; disease due to chromosome insertion; disease due to chromosome translocation; disease due to chromosome fusion; disease due to chromosome flipping; disease due to chromosome mutations; disease due defects in spatial localization of a chromosome; disease due to chromosome copy number (aneuploidy); disease due to gene copy number; disease due to gene deletion or insertion or polymorphism (mutation).

### Kits

In some embodiments, the technology provides a kit for imaging a nucleic acid. In some embodiments, a kit comprises: a) a DNA-targeting RNA or a nucleic acid comprising a nucleotide sequence encoding a DNA-targeting RNA, wherein the DNA-targeting RNA comprises: i) a first segment comprising a nucleotide sequence that is complementary to a target sequence in the target DNA; and a second segment that interacts with a polypeptide to form an RNP as described herein; and, optionally, b) a buffer. In some embodiments, a kit further comprises a nucleic acid comprising a nucleotide sequence encoding a variant Cas9 polypeptide that exhibits minimized, reduced, or eliminated nuclease activity relative to wild-type Cas9 (e.g., a dCas9). In some embodiments, a kit further comprises a variant Cas9 polypeptide that exhibits reduced, minimized, undetectable, and/or no nuclease activity relative to wild-type Cas9 (e.g., a dCas9). In some embodiments, a kit further includes one or more additional reagents, where such additional reagents can be selected from: a buffer; a wash buffer; a control reagent; a control expression vector or RNA polynucleotide; a reagent for in vitro production of the Cas9 polypeptide from DNA; and the like. In some embodiments, the Cas9 polypeptide included in a kit is a fusion protein comprising a Cas9 or a dCas9. In some embodiments, the fusion protein comprises a domain providing enhanced or improved localization (e.g., transport) to the nucleus (e.g., an NLS, an IBB, etc.) In some embodiments, components of the kit are in separate container; in some embodiments, one or more components of a kit are combined in a single container. Further, in some embodiments, a kit can further include instructions for using the components of the kit to practice a method described herein.

In some embodiments, the technology provides a kit for isolating components involved with chromosomal interactions at a target locus. In some embodiments, kits comprise one or more compositions as described herein, e.g., packaged in one or more containers for use by a user.

In some embodiments, a kit comprises: a) a labeled DNA-targeting RNA or a nucleic acid comprising a nucleotide sequence encoding a DNA-targeting RNA, wherein the DNA-targeting RNA comprises: i) a first segment comprising a nucleotide sequence that is complementary to a target sequence in the target DNA; and ii) a second segment that interacts with a polypeptide to form an RNP as described herein; and, optionally, b) a buffer. In some embodiments, a kit further comprises a nucleic acid comprising a nucleotide sequence encoding a variant Cas9 polypeptide or CRISPR enzyme that exhibits minimized, reduced, or eliminated nuclease activity relative to wild-type Cas9 (e.g., a dCas9). In some embodiments, a kit further comprises a variant Cas9 polypeptide that exhibits reduced, minimized, undetectable, and/or no nuclease activity relative to wild-type Cas9 (e.g., a dCas9). In some embodiments, a kit further includes one or more additional reagents, where such additional reagents can be selected from: a lysis buffer; a binding buffer; a wash buffer; a control reagent; a control expression vector or RNA polynucleotide; a reagent for in vitro production of the Cas9 polypeptide from DNA; and the like. In some embodiments, the Cas9 polypeptide or CRISPR enzyme included in a kit is a fusion protein comprising a Cas9 or a dCas9. In some embodiments, the fusion protein comprises a domain providing enhanced or improved localization (e.g., transport) to the nucleus (e.g., an NLS, an IBB, etc.) In some embodiments, components of the kit are in separate container; in some embodiments, one or more components of a kit are combined in a single container. Further, in some embodiments, a kit can further include instructions for using the components of the kit to practice a method described herein.

### Systems

Some embodiments of the technology provide systems for imaging (e.g., identifying, detecting) a nucleic acid. Systems according to the technology comprise, e.g., polypeptides (e.g., Cas9, dCas9, or the like or modified variants thereof) and RNAs (e.g., dgRNA, sgRNA). Related embodiments provide expression systems (e.g., comprising nucleic acids encoding the polypeptides and/or RNAs; and one or more expression hosts) for producing polypeptides and/or RNAs described herein using an in vitro system. In some embodiments, the systems further comprise an in-vitro system for assembly of RNP complexes. Some embodiments comprise fluid handling (e.g., in some embodiments, microfluidics) components for transporting samples, reagents, and other compositions for imaging a nucleic acid with a RNP. Some embodiments comprise components for fluid storage and fluid waste storage. In some embodiments, one or more components is/are provided to the system in the form of a kit.

In some embodiments, systems comprise a cell (e.g., a cultured cell, a primary cell, e.g., a cell in a sample obtained from a subject). For example, in some embodiments systems comprise a cell comprising an RNP as described herein. In particular embodiments, systems comprise a cell, a polypeptide, and one or more RNA molecules, e.g., a cell comprising a cell comprising a detectably labeled polypeptide (e.g., a Cas9 or dCas9 polypeptide) and/or a detectably labeled RNA (e.g., a sgRNA, a dgRNA (e.g., a crRNA and tracrRNA)). In some embodiments, a cell comprises a plurality of detectably labeled RNP complexes. In some embodiments, a cell comprises a plurality of detectably labeled RNP complexes comprising distinguishable detectable labels.

Some embodiments further comprise a fluorescence microscope comprising an illumination configuration to excite detectable labels. Some embodiments comprise a fluorescence detector, e.g., a detector comprising an intensified charge coupled device (ICCD), an electron-multiplying charge coupled device (EM-CCD), a complementary metal-oxide-semiconductor (CMOS), a photomultiplier tube (PMT), an avalanche photodiode (APD), and/or another detector capable of detecting fluorescence emission from single chromophores. Some embodiments comprise a computer and software encoding instructions for the computer to perform.

For example, in some embodiments, computer-based analysis software is used to translate the raw data generated by the imaging (e.g., the presence, absence, amount, or identity) of one or more nucleic acids into data of predictive value for a clinician. The clinician can access the predictive data using any suitable means.

For instance, some embodiments comprise a computer system upon which embodiments of the present technology may be implemented. In various embodiments, a computer system includes a bus or other communication mechanism for communicating information and a processor coupled with the bus for processing information. In various embodiments, the computer system includes a memory, which can be a random access memory (RAM) or other dynamic storage device, coupled to the bus, and instructions to be executed by the processor. Memory also can be used for storing temporary variables or other intermediate information during execution of instructions to be executed by the processor. In various embodiments, the computer system can further include a read only memory (ROM) or other static storage device coupled to the bus for storing static information and instructions for the processor. A storage device, such as a magnetic disk or optical disk, can be provided and coupled to the bus for storing information and instructions.

In various embodiments, the computer system is coupled via the bus to a display, such as a cathode ray tube (CRT) or a liquid crystal display (LCD), for displaying information to a computer user. An input device, including alphanumeric and other keys, can be coupled to the bus for communicating information and command selections to the processor. Another type of user input device is a cursor control, such as a mouse, a trackball, or cursor direction keys for communicating direction information and command selections to the processor and for controlling cursor movement on the display. This input device typically has two degrees of freedom in two axes, a first axis (e.g., x) and a second axis (e.g., y), that allows the device to specify positions in a plane.

A computer system can perform embodiments of the present technology. Consistent with certain implementations of the present technology, results can be provided by the computer system in response to the processor executing one or more sequences of one or more instructions contained in the memory. Such instructions can be read into the memory from another computer-readable medium, such as a storage device. Execution of the sequences of instructions contained in the memory can cause the processor to perform the methods described herein. Alternatively, hard-wired circuitry can be used in place of or in combination with software instructions to implement the present teachings. Thus, implementations of the present technology are not limited to any specific combination of hardware circuitry and software.

For example, some embodiments of the technology are associated with (e.g., implemented in) computer software and/or computer hardware. In one aspect, the technology relates to a computer comprising a form of memory, an element for performing arithmetic and logical operations, and a processing element (e.g., a microprocessor) for executing a series of instructions (e.g., a method as provided herein) to read, manipulate, and store data. In some embodiments, a microprocessor is part of a system for collecting image data (e.g., a series of images, a movie, etc.) and processing image data to determine the presence, absence, amount, or identity of one or more nucleic acids in a sample.

Some embodiments comprise a storage medium and memory components. Memory components (e.g., volatile and/or nonvolatile memory) find use in storing instructions (e.g., an embodiment of a process as provided herein) and/or data (e.g., an image, a series of images, processed images, results describing the presence, absence, amount, or identity of one or more nucleic acids in a sample). Some embodiments relate to systems also comprising one or more of a CPU, a graphics card, and a user interface (e.g., comprising an output device such as display and an input device such as a keyboard).

Programmable machines associated with the technology comprise conventional extant technologies and technologies in development or yet to be developed (e.g., a quantum computer, a chemical computer, a DNA computer, an optical computer, a spintronics based computer, etc.).

In some embodiments, the technology comprises a wired (e.g., metallic cable, fiber optic) or wireless transmission medium for transmitting data. For example, some embodiments relate to data transmission over a network (e.g., a local area network (LAN), a wide area network (WAN), an ad-hoc network, the internet, etc.). In some embodiments, programmable machines are present on such a network as peers and in some embodiments the programmable machines have a client/server relationship. For example, some embodiments provide systems in which a processor is remote from one or more other components of the system, e.g., to provide a system arranged in a cloud computing arrangement.

In some embodiments, data are stored on a computer-readable storage medium such as a hard disk, flash memory, optical media, a floppy disk, etc.

In some embodiments, the technology provided herein is associated with a plurality of programmable devices that operate in concert to perform a method as described herein. For example, in some embodiments, a plurality of computers (e.g., connected by a network) may work in parallel to collect and process data, e.g., in an implementation of cluster computing or grid computing or some other distributed computer architecture that relies on complete computers (with onboard CPUs, storage, power supplies, network interfaces, etc.) connected to a network (private, public, or the internet) by a conventional network interface, such as Ethernet, fiber optic, or by a wireless network technology.

For example, some embodiments provide a computer that includes a computer-readable medium. The embodiment includes a random access memory (RAM) coupled to a processor. The processor executes computer-executable program instructions stored in memory. Such processors may include a microprocessor, an ASIC, a state machine, or other processor, and can be any of a number of computer processors, such as processors from Intel Corporation of Santa Clara, Calif. and Motorola Corporation of Schaumburg, Ill. Such processors include, or may be in communication with, media, for example computer-readable media, which stores instructions that, when executed by the processor, cause the processor to perform the steps described herein.

Embodiments of computer-readable media include, but are not limited to, an electronic, optical, magnetic, or other storage or transmission device capable of providing a processor with computer-readable instructions. Other examples of suitable media include, but are not limited to, a floppy disk, CD-ROM, DVD, magnetic disk, memory chip, ROM, RAM, an ASIC, a configured processor, all optical media, all magnetic tape or other magnetic media, or any other medium from which a computer processor can read instructions. Also, various other forms of computer-readable media may transmit or carry instructions to a computer, including a router, private or public network, or other transmission device or channel, both wired and wireless. The instructions may comprise code from any suitable computer-programming language, including, for example, C, C++, C#, Visual Basic, Java, Python, Perl, Swift, and JavaScript.

Computers are connected in some embodiments to a network. Computers may also include a number of external or internal devices such as a mouse, a CD-ROM, DVD, a keyboard, a display, or other input or output devices. Examples of computers are personal computers, digital assistants, personal digital assistants, cellular phones, mobile phones, smart phones, pagers, digital tablets, laptop computers, internet appliances, and other processor-based devices. In general, the computers related to aspects of the technology provided herein may be any type of processor-based platform that operates on any operating system, such as Microsoft Windows, Linux, UNIX, Mac OS X, etc., capable of supporting one or more programs comprising the technology provided herein. Some embodiments comprise a personal computer executing other application programs (e.g., applications). The applications can be contained in memory and can include, for example, a word processing application, a spreadsheet application, an email application, an instant messenger application, a presentation application, an Internet browser application, a calendar/organizer application, and any other application capable of being executed by a client device.

All such components, computers, and systems described herein as associated with the technology may be logical or virtual.

In accordance with such a computer system, some embodiments of the technology provided herein further comprise functionalities for collecting, storing, and/or analyzing data (e.g., presence, absence, identity of a nucleic acid). For example, some embodiments contemplate a system that comprises a processor, a memory, and/or a database for, e.g., storing and executing instructions, analyzing fluorescence image data, performing calculations using the data, transforming the data, and storing the data. In some embodiments, an algorithm applies a statistical model to the data.

Many diagnostics involve determining the presence of, absence of, identity of, or a nucleotide sequence of, one or more nucleic acids. Thus, in some embodiments, an equation comprising variables representing the presence, absence, identity, concentration, amount, or sequence properties of multiple nucleic acids produces a value that finds use in making a diagnosis or assessing the presence or qualities of a nucleic acid. As such, in some embodiments this value is presented by a device, e.g., by an indicator related to the result (e.g., an LED, an icon on a display, a sound, or the like). In some embodiments, a device stores the value, transmits the value, or uses the value for additional calculations.

Thus, in some embodiments, the present technology provides the further benefit that a clinician, who is not likely to be trained in genetics or molecular biology, need not understand the raw data. The data are presented directly to the clinician in its most useful form. The clinician is then able to utilize the information to optimize the care of a subject. The present invention contemplates any method capable of receiving, processing, and transmitting the information to and from laboratories conducting the assays, information providers, medical personal, and/or subjects. For example, in some embodiments of the present technology, a sample is obtained from a subject and submitted to a profiling service (e.g., a clinical lab at a medical facility, genomic profiling business, etc.), located in any part of the world (e.g., in a country different than the country where the subject resides or where the information is ultimately used) to generate raw data. Where the sample comprises a tissue or other biological sample, the subject may visit a medical center to have the sample obtained and sent to the profiling center or subjects may collect the sample themselves and directly send it to a profiling center. Where the sample comprises previously determined biological information, the information may be directly sent to the profiling service by the subject (e.g., an information card containing the information may be scanned by a computer and the data transmitted to a computer of the profiling center using electronic communication systems). Once received by the profiling service, the sample is processed and a profile is produced that is specific for the diagnostic or prognostic information desired for the subject. The profile data are then prepared in a format suitable for interpretation by a treating clinician. For example, rather than providing raw expression data, the prepared format may represent a diagnosis or risk assessment for the subject, along with recommendations for particular treatment options. The data may be displayed to the clinician by any suitable method. For example, in some embodiments, the profiling service generates a report that can be printed for the clinician (e.g., at the point of care) or displayed to the clinician on a computer monitor. In some embodiments, the information is first analyzed at the point of care or at a regional facility. The raw data are then sent to a central processing facility for further analysis and/or to convert the raw data to information useful for a clinician or patient. The central processing facility provides the advantage of privacy (all data are stored in a central facility with uniform security protocols), speed, and uniformity of data analysis. The central processing facility can then control the fate of the data following treatment of the subject. For example, using an electronic communication system, the central facility can provide data to the clinician, the subject, or researchers. In some embodiments, the subject is able to access the data using the electronic communication system. The subject may chose further intervention or counselling based on the results. In some embodiments, the data are used for research use. For example, the data may be used to further optimize the inclusion or elimination of markers as useful indicators of a particular condition associated with the disease.

### Uses

The imaging technologies described herein find use in, e.g., imaging, diagnostics, and treatment of patients. Applications include research applications; diagnostic applications; industrial applications; and treatment applications. Research applications include, e.g., characterizing, detecting, and/or identifying nucleic acids in a cell (e.g., a living cell).

Further uses of embodiments of the technology described herein include one or more of the following: genome imaging; copy number analysis; analysis of living cells; detection of highly repetitive genome sequence or structure; detection of complex genome sequences or structures; detection of gene duplication or rearrangement; chromosomal labeling; large scale diagnostics of diseases and genetic disorders related to genome deletion, duplication, and rearrangement; use of multiple unique sgRNAs for high-throughput imaging and/or diagnostics; multicolor differential detection of target sequences; identification or diagnosis of diseases of unknown cause or origin; and 4-dimensional (e.g., time-lapse) or 5-dimensional (e.g., multicolor time-lapse) imaging of cells (e.g., live cells), tissues, or organisms.

Although the disclosure herein refers to certain illustrated embodiments, it is to be understood that these embodiments are presented by way of example and not by way of limitation.

### EXAMPLES

### Materials and Methods

### Plasmid Construction, Protein Purification, Cell Lines Construction

For expression of dCas9-GFP protein (see, e.g., FIG. 1), dCas9-GFP (e.g., comprising two copies of NLS-GFP) was subcloned from the pHR-TRE3G-dCas9-GFP plasmid (Chen (2013) Cell 155: 1479, incorporated herein by reference) into a pET-based bacterial expression vector containing an N-terminal His-MBP-TEV tag (Jinek et al. (2014) Science 343: 1247997, incorporated herein by reference). Protein purification was pursued using the protocol for dCas9 protein (Jinek et al., 2014). dCas9 protein was provided by Dr. Fuguo Jiang (Jennifer A. Doudna, University of California, Berkeley).

### Guide RNAs Synthesis, Purification and Preparation.

Unlabeled sgRNAs were synthesized using in vitro transcription (HiScribe™ T7 Quick High Yield RNA Synthesis Kit, NEB). DNA templates comprised a T7 promoter binding sequence and sequences encoding full length sgRNAs. sgRNAs were and purified with RNA Clean & Concentrator™-100 (Zymo Research, Irvine, Calif.). Unlabeled tracrRNAs were synthesized and purified in the same way. Fluorescently labeled crRNAs and tracrRNAs were synthesized by Integrated DNA Technologies (Redwood City, Calif.). Purified sgRNAs were refolded in 1× folding buffer (20 mM HEPES, pH 7.5; 150 mM KCl), then incubated at 70° C. for 1 minute and, gradually cooled to room temperature. Then 1 mM MgCl₂ was added and the solution was incubated at 40° C. for 5 minutes. The same refolding procedures were used to anneal crRNA and tracr RNAs at an equal (e.g., 1:1) molar ratio in 1× folding buffer.

In these experiments, the RNAs comprised the following sequences (5′-3′):

tracrRNA
(SEQ ID NO: 2)
rGrGrArArCrCrArUrUrCrArArArArCrArGrCrArUrArGrCrAr
ArGrUrUrUrArArArUrArArGrGrCrUrArGrUrCrCrGrUrUrArU
rCrArArCrUrUrGrArArArArArGrUrGrGrCrArCrCrGrArGrUr
CrGrGrUrGrCrUrUrUrUrUrUrU
crRNA targeting chromosome 3
(SEQ ID NO: 3)
rUrGrArUrArUrCrArCrArGrGrUrUrUrArArGrArGrCrUrArUr
GrCrUrGrUrUrUrUrG
crRNA targeting chromosome 13
(SEQ ID NO: 4)
rArCrCrArUrUrCrCrUrUrCrGrUrUrUrArArGrArGrCrUrArUr
GrCrUrGrUrUrUrUrG
sgRNA targeting telomeres
(SEQ ID NO: 5)
rGrUrUrArGrGrGrUrUrArGrGrGrUrUrArGrGrGrUrUrArGrUr
UrUrArArGrArGrCrUrArUrGrCrUrGrGrArArArCrArGrCrArU
rArGrCrArArGrUrUrUrArArArUrArArGrGrCrUrArGrUrCrCr
GrUrUrArUrCrArArCrUrUrGrArArArArArGrUrGrGrCrArCrC
rGrArGrUrCrGrGrUrGrCrUrUrUrUrUrUrU
crRNA targeting telomeres
(SEQ ID NO: 7)
rGrUrUrArGrGrGrUrUrArGrGrGrUrUrArGrGrGrUrUrArGrUr
UrUrArArGrArGrCrUrArUrGrCrUrGrUrUrUrUrG

The “r” in these sequences denotes a ribonucleotide at this position, a ribonucleotide comprising adenine (rA), guanine (rG), cytosine (rC), or uracil (rU). In some embodiments, the RNA molecules comprise a detectable label, e.g., a fluorescent dye, linked to the 5′ end of the RNA molecule.

In these experiments, the Cas9 protein (NLS-dCas9-NLS-EFFP) comprised the following sequence:

(SEQ ID NO: 8)
GAASPGPKKKRKVDKKYSIGLAIGTNSVGWAVITDEYKVPSKKFKVLGNT
DRHSIKKNLIGALLFDSGETAEATRLKRTARRRYTRRKNRICYLQEIFSN
EMAKVDDSFFHRLEESFLVEEDKKHERHPIFGNIVDEVAYHEKYPTIYHL
RKKLVDSTDKADLRLIYLALAHMIKFRGHFLIEGDLNPDNSDVDKLFIQL
VQTYNQLFEENPINASGVDAKAILSARLSKSRRLENLIAQLPGEKKNGLF
GNLIALSLGLTPNFKSNFDLAEDAKLQLSKDTYDDDLDNLLAQIGDQYAD
LFLAAKNLSDAILLSDILRVNTEITKAPLSASMIKRYDEHHQDLTLLKAL
VRQQLPEKYKEIFFDQSKNGYAGYIDGGASQEEFYKFIKPILEKMDGTEE
LLVKLNREDLLRKQRTFDNGSIPHQIHLGELHAILRRQEDFYPFLKDNRE
KIEKILTFRIPYYVGPLARGNSRFAWMTRKSEETITPWNFEEVVDKGASA
QSFIERMTNFDKNLPNEKVLPKHSLLYEYFTVYNELTKVKYVTEGMRKPA
FLSGEQKKAIVDLLFKTNRKVTVKQLKEDYFKKIECFDSVEISGVEDRFN
ASLGTYHDLLKIIKDKDFLDNEENEDILEDIVLTLTLFEDREMIEERLKT
YAHLFDDKVMKQLKRRRYTGWGRLSRKLINGIRDKQSGKTILDFLKSDGF
ANRNFMQLIHDDSLTFKEDIQKAQVSGQGDSLHEHIANLAGSPAIKKGIL
QTVKVVDELVKVMGRHKPENIVIEMARENQTTQKGQKNSRERMKRIEEGI
KELGSQILKEHPVENTQLQNEKLYLYYLQNGRDMYVDQELDINRLSDYDV
DAIVPQSFLKDDSIDNKVLTRSDKNRGKSDNVPSEEVVKKMKNYWRQLLN
AKLITQRKFDNLTKAERGGLSELDKAGFIKRQLVETRQITKHVAQILDSR
MNTKYDENDKLIREVKVITLKSKLVSDFRKDFQFYKVREINNYHHAHDAY
LNAVVGTALIKKYPKLESEFVYGDYKVYDVRKMIAKSEQEIGKATAKYFF
YSNIMNFFKTEITLANGEIRKRPLIETNGETGEIVWDKGRDFATVRKVLS
MPQVNIVKKTEVQTGGFSKESILPKRNSDKLIARKKDWDPKKYGGFDSPT
VAYSVLVVAKVEKGKSKKLKSVKELLGITIMERSSFEKNPIDFLEAKGYK
EVKKDLIIKLPKYSLFELENGRKRMLASAGELQKGNELALPSKYVNFLYL
ASHYEKLKGSPEDNEQKQLFVEQHKHYLDEIIEQISEFSKRVILADANLD
KVLSAYNKHRDKPIREQAENIIHLFTLTNLGAPAAFKYFDTTIDRKRYTS
TKEVLDATLIHQSITGLYETRIDLSQLGGDEGAPKKKRKVGSSVSKGEEL
FTGVVPILVELDGDVNGHKFSVSGEGEGDATYGKLTLKFICTTGKLPVPW
PTLVTTLTYGVQCFSRYPDHMKQHDFFKSAMPEGYVQERTIFFKDDGNYK
TRAEVKFEGDTLVNRIELKGIDFKEDGNILGHKLEYNYNSHNVYIMADKQ
KNGIKVNFKIRHNIEDGSVQLADHYQQNTPIGDGPVLLPDNHYLSTQSAL
SKDPNEKRDHMVLLEFVTAAGITLGMDELYK*

The dCas9-EGFP polypeptide comprised 1631 amino acids and had a moleculal weight of approximately 187655.65.

### Cell Culture

U2OS cells and human retinal pigment epithelium (RPE) cells were cultured in DMEM with GlutaMAX1 (Life Technologies) in 10% Tet-system-approved FBS (Life Technologies). Two lines of Amnio fluid cells, AF12070 (Trisomy 13) and GM0950 (apparently normal cell), were obtained from Coriell cell center, cultured in AminoMaxII with 10% Tet-system-approved FBS. Human primary T cells (peripheral Blood CD3+ Pan T Cells) were purchased from Stem Cell Express (Placerville, Calif.), activated using Dynabeads Human T-Activator CD3/CD28 beads (Gibco, Life Technologies), and cultured in X-VIVO 15 (Lonza Cat #BE02-053Q). All cells were maintained at 37° C. and 5% CO₂ in a humidified incubator.

### Delivery of Fluorescent RNP Complexes

fRNP delivery was performed using the Neon 10-μl transfection kit (Thermo fisher, MPK1025) (Liang et al. (2015), J Biotechnol 208: 44-53, incorporated herein by reference); transformed cells were plated in 24 well plates. The plates can be pretreated with 50-200 μg/ml collagen for 2 hours at 37° C. to accelerate cell attachments. To increase transfection efficacy, cells can be pretreated with nocodazole for 16 hours before transfection. For RNP transfection using dCas9-GFP and in vitro purified sgRNAs, 11-22 pmol dCas9-GFP was mixed with a 4:1 molar ratio of sgRNAs in buffer R and then incubated at room temperature for 10 minutes to allow RNP assembly. For RNP delivery of dye-labeled crRNA/tracr RNA complexes, 10-25 pmol of each labeled crRNA and/or tracrRNA were mixed with an equal amount of dCas9-GFP or dCas9 in transfection buffer R or T and then incubated at room temperature for 10 minutes to allow RNP assembly. The assembled RNP complexes were transfected into 1-2×10⁵ suspended cells using standard Neon 10-μl transfection kit (Thermo Fisher MPK1025). Electroporation was performed in U2OS cells at 1400 V for 15 ms (up to 4 pulses); in RPE cells at 1350 V for 20 ms (up to 2 pulses); in T cells at 1400 V for 10 ms (up to 3 pulses); and in AFSCs at 1400 V for 30 ms (up to 4 pulses. The transfected cells were immediately plated into 24-well ibidi μ-plate plates containing pre-warmed culture medium for imaging.

For lipid-mediated transfection of fluorescent RNP complex, U2OS cells were placed in 24-well ibidi plates to reach approximately 50% confluency prior to (e.g., one day before) transfection. For each well, a transfection mixture comprising 1-5 pmol of fluorescent crRNA:tracrRNA complex and an equal amount of Cas9 in 25 μl Opti-MEM I reduced serum media (ThermoFisher, 31985062) was mixed and incubated at room temperature for 10 minutes. After incubation, 3 μl of Lipofectamine RNAiMax transfection reagent (ThermoFisher, 13778030) in 25 μl Opti-MEM I was added the transfection mixture, mixed, and incubated at room temperature for 15 minutes. The final transfection mixture was then added to each well containing the U2OS cells. Cells were imaged 12-24 hours after transfection.

### Microscopy and Image Analysis

Microscopy was performed under a Nikon TiE inverted microscope equipped with Andor iXon Ultra-897 EM-CCD cameras and 100*PLAN APO oil objective (NA=1.49). Image analysis was performed in imageJ or metamorph.

### In Vivo Capture of Locus Specific Chromatin Complex by Guide RNA Affinity Purification

Biotin-labeled guide RNA were synthesized and delivered into the cells using RNP delivery methods described above. Cells were cultured in 37° C. for 16 hours to recover. Cells were collected, fixed in formaldehyde, and lysed in lysis buffer. Next, nuclei were isolated by centrifugation and sonicated to create chromatin fragments. After ultracentrifugation, the supernatant was incubated with Streptavidin beads for affinity purification of the bound chromatin complexes. The isolated locus-specific complexes were analysed using mass spectrometry to identify proteins, DNA, and/or RNA components associated with the target regions. qPCR was performed to confirm proper isolation of genomic targets.

### Example 1

### DNA-Encoded dCas9-EGFP Imaging is Not Suitable for Diagnosis Imaging

Previous methods of dCas9-based imaging used dCas9-EGFP fusions and a sequence-specific sgRNA both expressed in cells from lentiviral vectors. These techniques were based on first establishing a system for stably expressing dCas9-EGFP in cells to enhance imaging efficiency to an acceptable level (Chen et al. (2013). Dynamic imaging of genomic loci in living human cells by an optimized CRISPR/Cas system. Cell 155, 1479-1491, incorporated herein by reference; Chen et al. (2016). Expanding the CRISPR imaging toolset with Staphylococcus aureus Cas9 for simultaneous imaging of multiple genomic loci. Nucleic Acids Res. 44(8):e75, incorporated herein by reference). Such systems, however, are not suitable for diagnostic techniques comprising imaging primary cells.

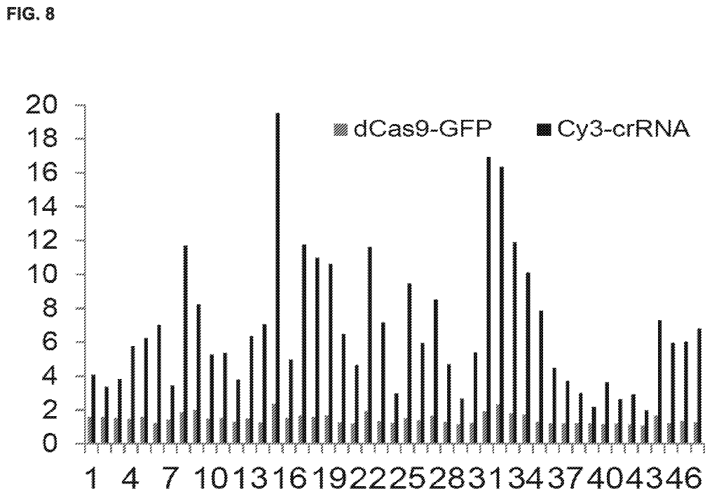

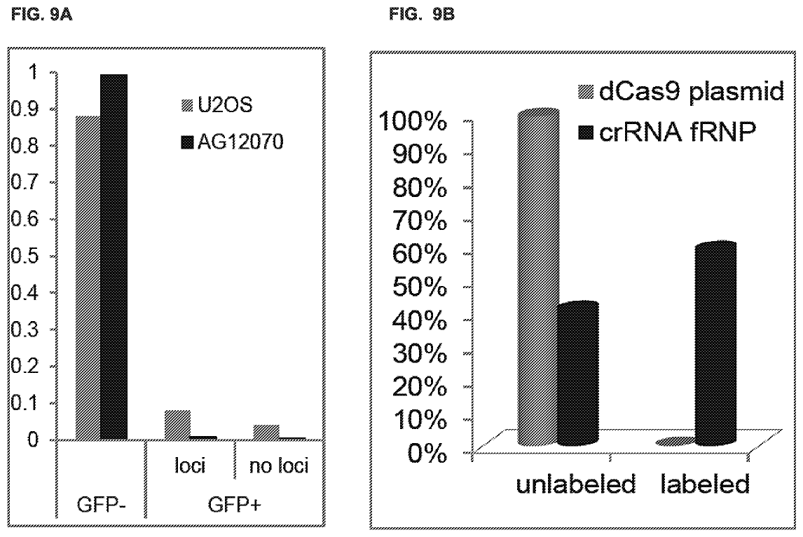

Thus, during the development of the technology described herein, it was contemplated that transient transfection of plasmids encoding dCas9-EGFP and sgRNA may provide a solution for imaging live primary cells. Accordingly, experiments were conducted in which two plasmids were co-transfected into cells from a human bone osteosarcoma epithelial cell line (U2OS) and Patau Syndrome patient-derived amniotic fluid cells (AG12070) (FIG. 9A). One of the plasmids encoded dCas9-EGFP and the other plasmid encoded an sgRNA that targets the repetitive sequence within chromosome 13 that provides for the detection of chromosome 13 (Ma et al. (2016). Multiplexed labeling of genomic loci with dCas9 and engineered sgRNAs using CRISPRainbow. Nat Biotechnol 34, 528-530, incorporated herein by reference). A leaky dox-inducible TRE3G promoter was used to drive dCas9-GFP expression to adequate levels for imaging with acceptable background noise (Chen et al. (2013). Dynamic imaging of genomic loci in living human cells by an optimized CRISPR/Cas system. Cell 155, 1479-1491, incorporated herein by reference; Chen et al. (2016). Expanding the CRISPR imaging toolset with Staphylococcus aureus Cas9 for simultaneous imaging of multiple genomic loci. Nucleic Acids Res. 44(8):e75, incorporated herein by reference).

After co-transfection of the two plasmids, only 12% of cells were observed to express dCas9-GFP from the leaky promoter and the expression level varied widely among expressing cells. Importantly, an excessive number of aggregated complexes were observed in the nucleus. Thus, the efficiency of imaging chromosome 13 was low (8% in U2OS cells) with unacceptable amounts of aggregation in the nucleus (FIG. 9A, GFP+, grey bar).

In additional experiments conducted during the development of the technology described herein, the two plasmids described above were co-transfected into trisomy 13 patient-derived amniotic fluid cells (AG12070). In these experiments, the transfection efficiency was extremely low (less than 1% of cells expressed the dCas9-GFP) (FIG. 9A, GFP+, black bar). Further, correct chromosome 13 signals were rarely observed among the transfected cells and the formation of aggregation complexes in the nucleus was again a major problem. Accordingly, these experiments indicate that methods based on dCas9-EGFP and DNA-encoding systems are not suitable for robust diagnostic applications in either cell lines or primary cells. Therefore, experiments were conducted to test alternative imaging methods that provide diagnostic imaging in primary cells.

### Example 2

### Ribonucleoprotein-Based Imaging in Living Cells

Some applications of imaging, such as clinical diagnostic imaging, would benefit from rapid and sensitive methods to image nucleic acids in primary cells. During the development of embodiments of the technology described herein, it was contemplated to use a ribonucleoprotein (RNP) (e.g., comprising Cas9 and guide RNA) coupled to fluorescent dyes to produce a fluorescent RNP (fRNP) for imaging applications.

During the development of the technology described herein, experiments were conducted that indicated that a CRISPR fRNP approach provides imaging in live cells. In particular, an RNP delivery method was developed for imaging live cells using fluorescently labeled dCas9 proteins or fluorescently labeled guide RNAs. First, a recombinant DNA construct was produced comprising a dCas9-EGFP fusion protein coupled to two copies of a nuclear localization signal (NLS) from a bacterial expression system. The dCas9-EGFP construct was used to express the dCas9-EGFP protein (comprising the NLS) in E coli and the dCas9-EGFP protein (comprising the NLS) was purified in vitro. Next, an sgRNA targeting human telomeres (“sgTel”) was transcribed and purified in vitro. Finally, the purified dCas9-EGFP protein (comprising the NLS) and the sgRNA were assembled to form RNP complexes in vitro. These RNPs were transfected by electroporation into a U2OS cell line expressing TRF1-mCherry to label telomeres.

During these experiments, image data were collected to record the signals from the EGFP and mCherry fluorescent labels. The images indicated localization of a large amount of dCas9-EGFP to telomeres after transfection; localization to telomeres was confirmed by co-localization of the EGFP signal with the TRF1-mCherry signal. Control experiments indicated that dCas9-EGFP assembled with a non-targeting sgRNA was distributed evenly throughout the nucleus and showed some amount of cytoplasmic aggregation. These results indicate that the dCas9-GFP RNP method provides specific genomic labeling. In particular, additional experiments were conducted during the development of embodiments of the technology in which dCas9-EGFP was used to label telomeres and the labeled telomeres were detected and monitored in retinal pigment epithelial (RPE) cells for a time period throughout cell division.

Next, experiments were conducted during the development of embodiments of the technology in which guide RNAs were synthesized in vitro to incorporate fluorescent dyes. The length of a CRISPR sgRNA exceeds 110 nucleotides, which is difficult and costly to synthesize. Accordingly, instead of using a conventional sgRNA, a two-RNA crRNA/tracrRNA complex was used for labeling (Jinek et al. (2012). A programmable dual-RNA-guided DNA endonuclease in adaptive bacterial immunity. Science 337, 816-821, incorporated herein by reference). Embodiments comprised using a crRNA/tracrRNA duplex in which the original crRNA and/or tracrRNA sequences were modified based on previously reported improvements to sgRNAs for imaging applications (Chen et al. (2013). Dynamic imaging of genomic loci in living human cells by an optimized CRISPR/Cas system. Cell 155, 1479-1491, incorporated herein by reference).

In particular, experiments were conducted to characterize and compared the use of fluorescent labeled dCas9 or crRNA for genomic detection. In particular, experiments were conducted to label telomeres using RNP complexes comprising dCas9 and a crRNA/tracrRNA duplex, e.g., the sequence-modified crRNA/tracrRNA described above. First, a Cy3-labeled crRNA comprising a sequence to target telomeres (Cy3-crRNA^{Tel}) was chemically synthesized and used with an in vitro transcribed and purified tracrRNA. The Cy3-crRNA^{Tel} was annealed with the tracrRNA to form a fluorescent Cy3-labeled crRNA^{Tel}/tracrRNA dual-guide RNA (“dgRNA”). The Cy3-crRNA^{Tel}/tracrRNA dgRNA complex was mixed with dCas9-EGFP in vitro to assemble the RNP complex in vitro (see, e.g., FIG. 1). The RNP was introduced into nocodazole-synchronized U2OS cells using electroporation.

Data were collected that indicated labeling of telomeres in both dCas9-GFP and Cy3 crRNA^{Tel} channels. In a control experiment, a Cy3-labeled crRNA without a targeting sequence was not observed to label any loci. Importantly, the data indicated that the telomere labeling in the Cy3-crRNA channel was generally better than the telomere labeling in the dCas9-GFP channel; also, the telomere labeling in the Cy3-crRNA channel had less background than the telomere labeling in the dCas9-GFP channel.

### Example 3

### Characterization and Comparison of fRNP Methods

During the development of embodiments of the technology described herein, additional experiments were conducted to systematically characterize and compared the use of fluorescent labeled dCas9 or crRNA for genomic detection and imaging. In these experiments, the methods were used to image chromosome 3 (Chr3). In particular, a Cy3-labeled crRNA designed to target repetitive sequences within chromosome 3 was chemically synthesized. Next, the Cy3-crRNACh3 was annealed to tracrRNA in vitro and used to form RNP complexes with dCas9-EGFP protein (see, e.g., FIG. 1). The RNPs were transfected into U2OS cells by electroporation. Observation of the fluorescent channels indicated again that the Cy3-crRNA channel provided a better imaging signal than the dCas9-GFP channel.

In addition, analysis of the image data indicated that the signal-to-background ratio was better for Cy3-crRNA relative to dCas9-EGFP. Intensity linescans were plotted for genomic loci (see, e.g., FIG. 7). Signal to background ratios were calculated by dividing maximum fluorescence intensity of labeled genomic loci by average fluorescence intensity in the nucleus. 47 loci in 17 cells were analyzed. In the Cy3-crRNA channel, the labeled chromosome 3 loci were observed to have a fluorescence intensity approximately 19.6-fold higher than the average background intensity (FIG. 8, black bars). In contrast, the maximum signal observed in the dCas9-GFP channel reached only 2.36-fold higher than the averaged background (FIG. 8, grey bars).

In labeled S/G2 phase cells, the dynamics of two duplicated chromosome loci in sister chromatids were detected in the Cy3-crRNA channel, which was barely detectable in the dCas9-GFP channel. On average, the signal-to-background ratio of the Cy3-crRNA channel was observed to be approximately 7-fold higher than the signal provided by dCas9-EGFP. Without being bound by theory, the improved signal-to-background ratio provided by the experiment system using labeled crRNA is likely due to the rapid elimination of unbound guide RNAs in the samples, which are generally unstable when not part of an RNP and are degraded quickly in the cellular environment (thus decreasing background in this system). In contrast, unbound/misfolded dCas9-GFP is much more stable and remains as background that masks the real signal.

Additionally, there may be an advantage of using chemical fluorescent dyes compared to EGFP, considering the intrinsic differences in properties (fluorescence quantum yield, stability, etc.) between organic dyes and fluorescent proteins. Finally, it was observed that dCas9-GFP is often found in the nucleolus region within the nucleus, which adds to the background noise; in contrast, the Cy3-crRNA was rarely observed to be present in the nucleolus region.

### Example 4

### Genomic Imaging by fRNP is Rapid and Persistent

Additionally, experiments were conducted during the development of embodiments of the technology described herein to monitor the dynamics of Cy3-crRNA labeling over time, e.g., at a number of time points, to acquire data describing the kinetics of crRNA labeling. Using dCas9-GFP and Cy3-crRNA targeting loci on chromosome 3, the data collected during the experiments indicated that Cy3-crRNA is rapidly recruited to its genomic target, e.g., within 1 hour after transfection. In addition, the Cy3-crRNA labeled its target before cell attachment and the label persists at its target for longer than 72 hours. Using dCas9-GFP with unlabeled sgRNA targeting telomeres, dCas9-GFP is recruited to telomeres within 20 minutes after transfection into cells, which was confirmed by its co-localization with TRF1-mcherrry. Furthermore, the dCas9-GFP telomere labeling lasts for longer than 1 day. The data also indicated that the fluorescent crRNA-based genomic imaging produced the best signal within 24 hours after transfection.

Despite of the process of degradation that removes the crRNA molecules, crRNA-mediated genomic imaging within the nucleus remains stable for 72 hours after transfection. Accordingly, the persistent labeling by Cy3-crRNA is surprising, indicating that the labeled crRNAs are highly protected within the RNP complexes.

### Example 5

### Dynamic Tracking of Multiple Genomic Loci Using RNP Imaging

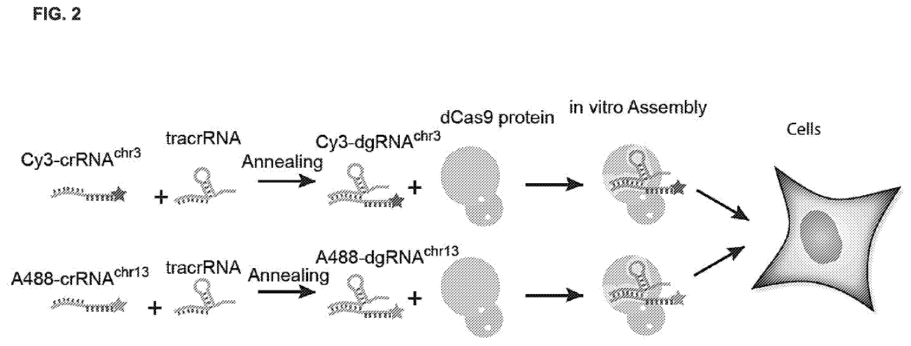

In some embodiments, fluorescently labeled crRNA provides an improved technology for multiplexed genomic tracking of multiple loci. During the development of embodiments of the technology, experiments were conducted to test use of multiple crRNAs labeled with different (distinguishable) fluorescent dyes to track the dynamics of multiple genomic loci simultaneously within the same cells. In these experiments, Cy3-crRNA was designed and synthesized to target chromosome 3 (Cy3-crRNA^{Chr3}) (see, e.g., FIG. 2, top dgRNA complex) and Atto488-crRNA was designed and synthesized to target chromosome 13 (Atto488-crRNA^{Chr13}) (see, e.g., FIG. 2, bottom dgRNA complex). The two crRNAs were annealed with tracrRNA to form two dgRNAs that were subsequently assembled with purified dCas9 protein in vitro to form RNP complexes. The RNP complexes were transfected into U2OS cells using electroporation. See, e.g., FIG. 2.

Data collected during these experiments indicated that both loci were labeled in the nucleus of the same cell after transfection. Chromosome 3 loci were labeled by Cy3-crRNA^{Chr3} and chromosome 13 loci were labeled by Atto488-crRNA^{Chr13}. In addition, both labels were tracked in space over time, thus providing time-resolved image data for multiple loci describing the dynamic movement of multiple chromosome loci in the nucleus.

Additional experiments were conducted to confirm that the fluorescent crRNAs were recruited to their genomic loci by dCas9. In these experiments, RNP complexes were assembled from purified dCas9-GFP and either: a) Cy5-crRNA^{Chr13}:tracrRNA; or b) Cy3-crRNA^{Chr3}:tracrRNA. Each of the two complexes was transfected into U2OS cells. The data collected indicated that dCas9-GFP co-localized with chromosome 3 loci labeled by Cy3-crRNA^{Chr3} and that dCas9-GFP co-localized with chromosome 13 loci labeled by Cy5-crRNA^{Chr13}, thus confirming that the fluorescent crRNAs were recruited to their targeted loci by the CRISPR-Cas9 system. The Cy5-crRNA labeled loci were rapidly photo-bleached, which was likely due to the poor photostability of Cy5 in living cells (Altman et al. (2011). Cyanine fluorophore derivatives with enhanced photostability. Nat Methods 9, 68-71, incorporated herein by reference). In contrast, the fluorescence of Cy3-crRNA and Atto488-crRNA remained stable.

### Example 6

### Multiplex Genomic Imaging

During the development of embodiments of the technology described herein, experiments were conducted to test the use of fluorescently labeled crRNAs to track the dynamics of endogenous loci in primary human cells. Primary human T lymphocytes are extensively studied for cancer immunotherapy and are highly difficult to transfect efficiently (see, e.g., Zhao (2006) Mol. Ther. 13: 151, incorporated herein by reference). Live-cell imaging in primary T lymphocytes is also challenging due to the compositions that are used to prepare cell suspensions of primary T lymphocytes.

First, experiments were conducted to test telomere labeling in primary primary human T lymphocytes using RNP complexes comprising dCas9-GFP and Cy3-crRNA^{tel}:tracrRNA or non-targeting Cy3-crRNA:tracrRNA. The RNP complexes were transfected into primary human T lymphocytes activated by a standard protocol. Imaging data indicated that telomere loci were labeled in cells transfected with the Cy3-crRNA^{tel}, while non-targeting Cy3-crRNA was observed to be distributed evenly throughout the nucleus. However, due to the rapid movement of T lymphocytes in suspension, resolving the dynamic movements of individual loci from time-lapse images is difficult.

Next, experiments were conducted to test multi-locus genomic imaging in primary human T lymphocytes using fRNP complexes. fRNPs comprising either purified dCas9 and Atto488-crRNA^{Chr13}:tracrRNA or purified dCas9 and Atto647/Cy3-crRNA^{Chr3}:tracrRNA were transfected into activated primary human T lymphocytes. The transfected cells were plated onto surfaces pre-coated with collagen to slow the movement of the T lymphocytes, thus causing a population of T lymphocytes to remain motionless over the time-course of genomic imaging. The imaging data indicated that two loci of chromosome 13 and two loci of chromosome 3 were labeled by fluorescent crRNAs in each nucleus. Further, the data indicated that movement of the two chromosomes were independently tracked using the time-lapse imaging. To distinguish crRNA-targeted loci in the nucleus from cytoplasmic aggregates, nuclei were stained with Hoechst 33342 during time-lapse imaging.

### Example 7

### Diagnosis of Patau Syndrome in Prenatal Amniotic Fluid Cells

During the development of embodiments of the technology provided herein, experiments were conducted to test use of the technology in cytogenetic studies of living cells obtained from a patient. Patau syndrome is a severe genetic disease caused by trisomy 13 chromosomal abnormalities, resulting in intellectual disability and physical impairment. For these experiments, prenatal amniotic fluid cells were obtained and cultured from a Patau syndrome (trisomy 13) patient and from a normal donor. The Patau and normal cells were transfected with fRNP complexes comprising purified dCas9 and Cy3-crRNA^{Chr13}. Data collected during these experiments indicated that Cy3-crRNA^{Chr13} was observed to target loci on chromosome 13 in a few cells, but that most of the Cy3-crRNA^{Chr13} formed large cytoplasmic aggregates in the mitochondria. Without being bound by theory, it was contemplated that the Cy3-crRNA^{Chr13} aggregated in the mitochondria because cyanine dye-coupled oligonucleotides in live cells are driven to mitochondria by a high membrane potential (see, e.g., Rhee (2010) Nucleic Acids Res. 38: e109, incorporated herein by reference).

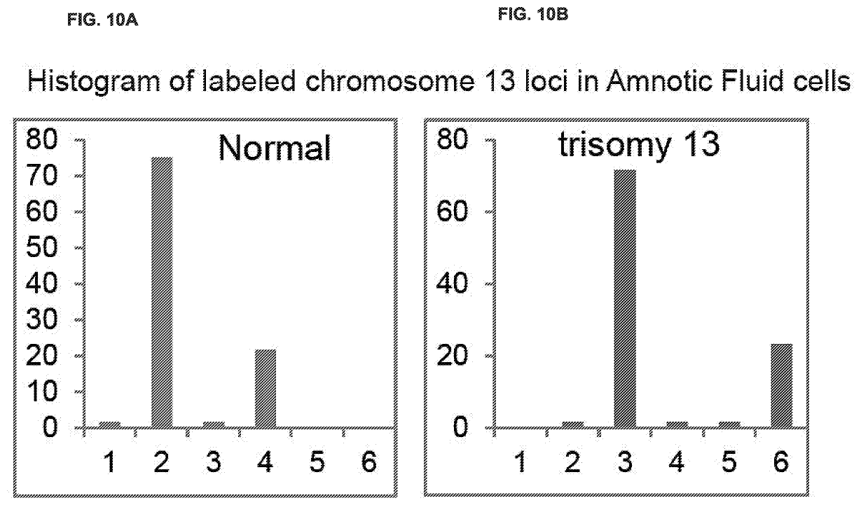

Accordingly, experiments were conducted during the development of embodiments of the technology to test conditions for avoiding the non-specific binding of dye to mitochondria. In particular, Atto565-crRNA^{Chr13} was used instead of the Cy3-crRNA^{Chr13}. Atto565-crRNA^{Chr13} was assembled with dCas9-GFP to provide fRNP complexes for transfection. Data were collected within 12 hours of transfection. The data collected indicated that chromosome 13 loci were clearly detected with Atto565-crRNA^{Chr13} in approximately 60% of living cells after transfection (FIG. 9B). Importantly, the number of labeled genomic loci was consistent with the number of chromosome 13 in each cell type (FIGS. 10A and 10B); Normal amniotic fluid cells showed either two dots (75% of cells) or four dots (22%), corresponding to normal amounts of chromosome 13; trisomy 13 amniotic fluid cells showed either 3 (72%) or 6 dots (23%) of chromosome 13, consistent with karyotyping results indicating aberrant numbers of chromosome 13 in these cells.

### Example 8

### Dynamic Imaging

Traditional cytogenetic studies are performed in fixed samples and thus do not provide observations or measurements describing the dynamic characteristics of the targets. In contrast, CRISPR/fRNP mediated fluorescent RNA targeting provides for the cytogenetic study of nucleic acids in living cells, e.g., in living primary cells.

During the development of embodiments of the technology provided herein, the dynamics of genomic loci were tracked by recording images of cells comprising RNPs over time (e.g., by recording a movie). In some embodiments, the dynamic measurements provide for differentiating true signals from false-positive signals. In particular, experiments were conducted to image chromosomes with resolution in the time domain. fRNP complexes were assembled and use to image chromosome 13 in live normal amniotic fluid cells.

In some amniotic fluid cells transfected with imaging RNPs, aggregations were detected in the cytoplasm in addition to actual signals (true positives) from genomic loci targeted by labeled RNPs (e.g., each comprising a fluorescent crRNA). In most experiments, aggregations can be distinguished from genomic targets by the shapes of labeled regions and their distances from the nucleus.

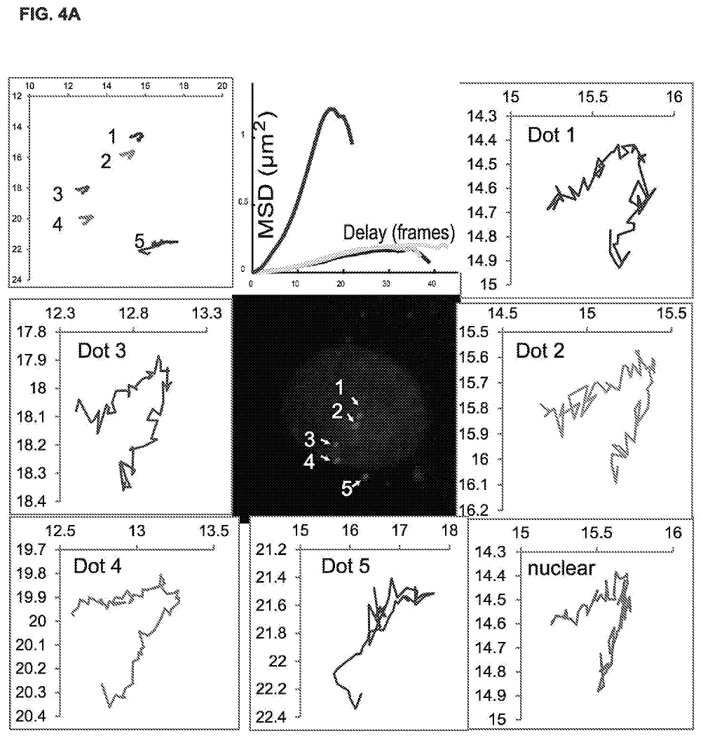

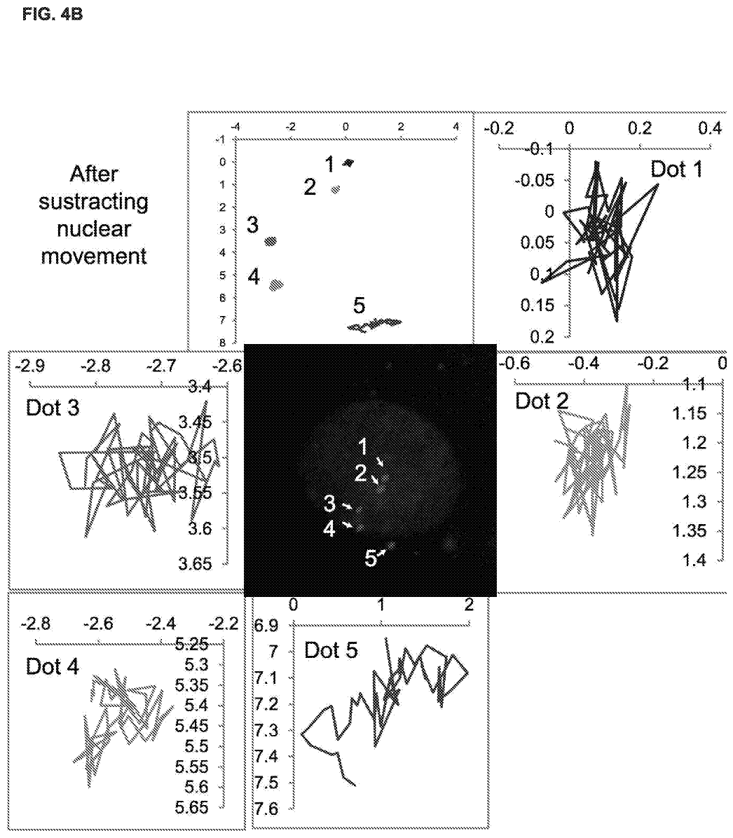

However, the aggregations occasionally exhibit a shape that is similar to targeted genomic loci and/or locate close to nucleus, thus hindering the proper identification of genomic loci relative to aggregates (e.g., dot 5 in FIGS. 4A and 4B).

Embodiments of the technology described herein comprise use of dynamic imaging to distinguish true signals (e.g., dots 1-4 in FIGS. 4A and 4B) at targeted loci from false positives produced by non-specific aggregates.

Data collected from experiments conducted during the development of the technology described herein indicated that genomic targets (dots 1-4 in FIG. 4A) follow the movements of nucleus and exhibit restricted localized movements relative to the directed nuclear movement (dots 1-4 in FIG. 4B). In contrast, a false-positive signal generated by aggregations (dot 5 in FIGS. 4A and 4B) moves randomly and often exhibits a higher mean square displacement rate than real targets (FIG. 4A).

It has also been observed that an aggregation invades the nucleus space and it would very likely be counted as false-positive signal using prior technologies, e.g., using fixed samples are used. However, in experiments conducted during the development of embodiments of the technology provided herein related to dynamic imaging, data indicated that a signal from an aggregate rapidly moved between nucleus and cytoplasm and should be removed from other true positive signals.

### Example 9

### Options for CRISPR-Mediated Imaging

In addition to using the fRNP approach to deliver fluorescently labeled crRNAs to label genomic loci in living cells, other possibilities were explored during the development of embodiments of the technology. For example, in addition to using fluorescently labeled crRNA, data were collected in experiments using a tracrRNA that was labeled with a fluorescent dye. Accordingly, in some embodiments, use of a fluorescently tracrRNA finds use in providing additional multiplexing capabilities. In addition, fluorescently labeled crRNA:tracrRNA complexes can be introduced into cells stably expressing dCas9 protein to achieve genomic imaging as well.

### Example 10

### On-Target DNA Binding Stabilizes gRNA within CRISPR Complexes

On-target DNA binding induces a conformational change in Cas9 protein (e.g., HNH domain re-localization) related to efficient nuclease activity and nucleic acid cleavage. Mismatches on target DNAs hinder such a conformational change, explaining the specificity of CRISPR gene editing. Accordingly, during the development of embodiments of the technology described herein, experiments were conducted to investigate if on-target DNA and mismatches on target DNAs also affect guide RNAs in CRISPR complexes.

Data collected from experiments conducted during the development of embodiments of the technology included biochemical evidence strongly indicating that on-target DNA binding stabilizes guide RNAs within CRISPR complexes in vitro, e.g., in an RNAse-rich environment, and this protection depends on dCas9 activity.

Previous studies suggested that gRNA are extremely unstable in cellular environments, which is a limiting factor for many CRISPR applications. Maintaining a high level of in vivo gRNA expression or increasing the stability of synthesized gRNA enhanced CRISPR-mediated gene editing and regulation.uring the development of embodiments of the technology described herein, experiments were conducted to test if on-target DNA binding protects CRISPR guide RNAs in cells (e.g., in vivo). For these experiments, two RNAs were synthesized: 1) a short Cy3-labeled sequence-specific crRNA (33 nt) comprising a 11-nt sequence targeting a repetitive sequence within chromosome 3 (see, e.g., Ma, H. et al. Multiplexed labeling of genomic loci with dCas9 and engineered sgRNAs using CRISPRainbow. Nat Biotechnol 34, 528-530 (2016)); and 2) an unlabeled tracrRNA (84 nt) (see, e.g., Jinek, M. et al. A programmable dual-RNA-guided DNA endonuclease in adaptive bacterial immunity. Science 337, 816-821 (2012); Chen, B. et al. Dynamic imaging of genomic loci in living human cells by an optimized CRISPR/Cas system. Cell 55, 1479-1491 (2013)).

crRNA targeting chromosome 3
(SEQ ID NO: 9)
UGAUAUCACAGGUUUAAGAGCUAUGCUGUUUUG
tracrRNA
(SEQ ID NO: 10)
GGAACCAUUCAAAACAGCAUAGCAAGUUUAAAUAAGGCUAGUCCGUUAUC
AACUUGAAAAAGUGGCACCGAGUCGGUGCUUUUUUU

The Cy3-labeled sequence-specific crRNA and unlabeled tracrRNA were annealed to form a crRNA:tracrRNA complex.

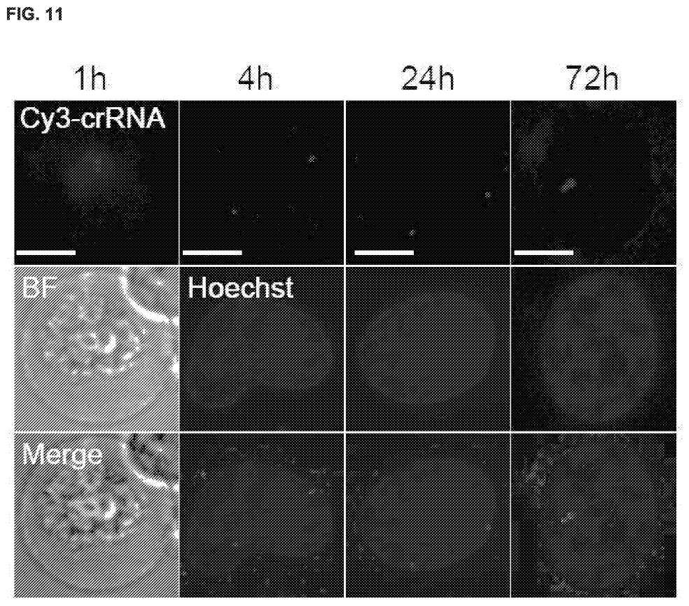

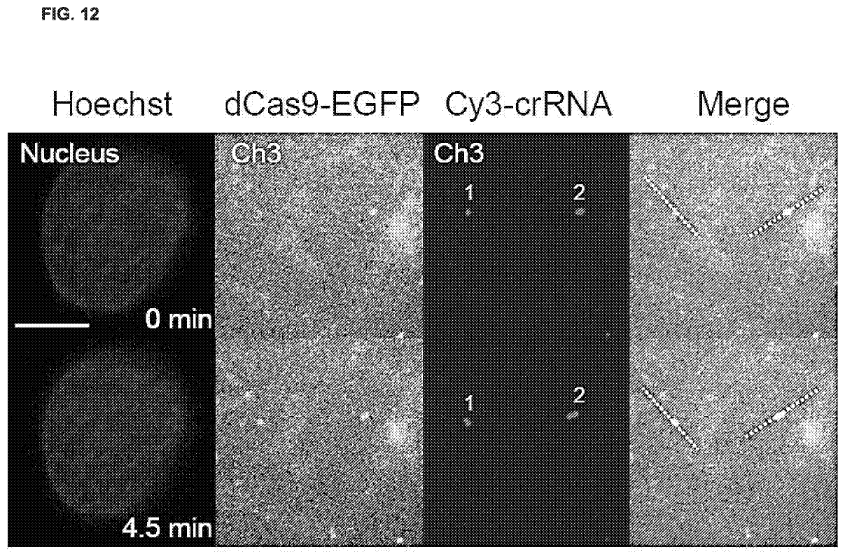

Next, a purified dCas9 or dCas9-EGFP protein was assembled with the Cy3-crRNA:tracrRNA complex in vitro. The assembled fluorescent ribonucleoprotein (fRNP) complex was delivered into U2OS cells by electroporation (see, e.g., Liang, X. et al. Rapid and highly efficient mammalian cell engineering via Cas9 protein transfection. J Biotechnol 208, 44-53 (2015)) (See, e.g., FIG. 1). Data collected during the experiments indicated that Cy3-crRNA was rapidly recruited to the Chromosome 3 genomic target after transfection and remained detectable in many cells after 72 hours (FIG. 11). These data indicate that the fluorescent CRISPR guide RNAs are very stable when bound to their on-target DNAs in the genome.

During these experiments, background fluorescence in the Cy3 channel was high immediately after electroporation and then gradually decreased, indicating that unbound fluorescent CRISPR guide RNAs were being degraded. However, experiments to quantify this observation were difficult due to cellular variations, photobleaching, transporting and binding process, and a variety of other factors.

To overcome this problem, experiments were conducted during the development of embodiments of the technology to compare fluorescence of the Cy3-labeled guide RNAs with the dCas9-GFP. Without being bound by theory, it was contemplated that the dCas9-GFP and Cy3-guide RNA channel may exhibit similar signal-to-background ratio if no other factors are involved because an equal molar ratio of dCas9-GFP and Cy3-guide RNAs were assembled into RNP complexes in vitro and then electroporated into the cells. Alternatively, it was contemplated that the guide RNA channel may exhibit higher signal to background ratio than dCas9-GFP channel if on-target DNA binding protects fluorescent guide RNAs within CRISPR complexes, while unbound guide RNAs are unstable.

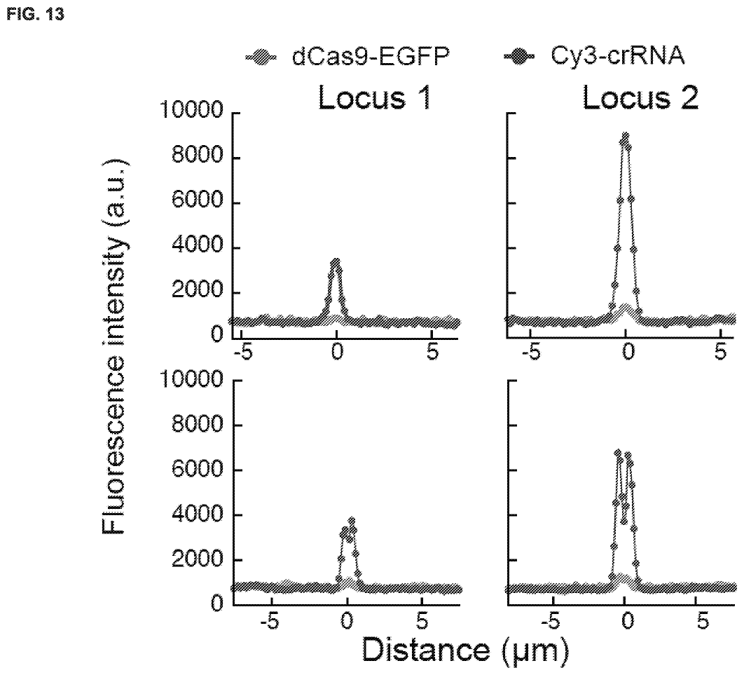

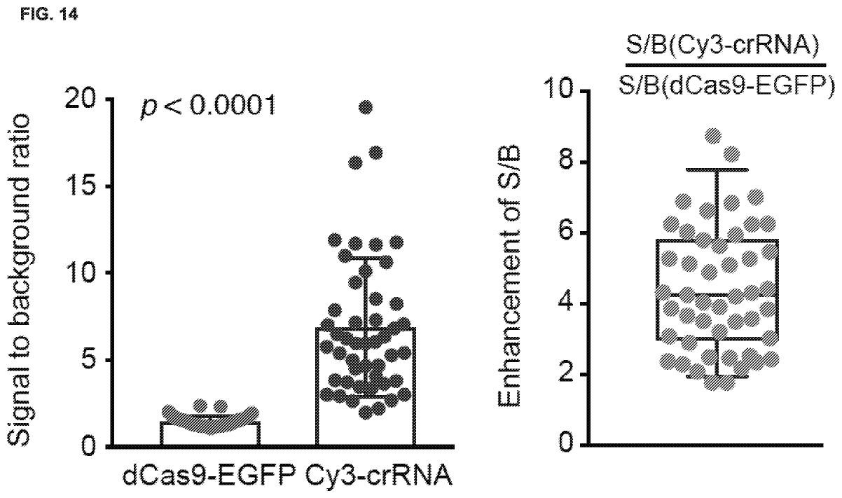

The data collected during these experiments indicated that the Cy3-crRNA channel exhibited greatly improved signal-to-background (S/B) ratio with significantly reduced background, compared to the dCas9-EGFP channel (FIG. 12). For example, the Cy3-crRNA labeled chromosome-3 loci showed a peak fluorescence intensity 12.6-fold higher than the background intensity, while dCas9-EGFP showed only an increase of 1.8-fold over background at the same loci (locus 2, 0 min, FIG. 12; FIG. 13). Quantification of 17 cells with 47 labeled chromosome-3 loci provided data indicating that the S/B ratio of Cy3-crRNA was 4.4±1.8 fold higher than dCas9-EGFP (t-test, p<0.0001) (FIG. 14). The higher S/B ratio of the Cy3-crRNA channel relative to the dCas9-GFP channel indicates that guide RNAs are selectively protected by on-target DNA binding in the genome.

Moreover, consistent with previous studies, accumulation of dCas9-GFP was observed in the nucleolus in many cells, but was not generally observed in the Cy3-crRNA channel. These data indicate that guide RNAs are not stable in other dCas9 accumulations other than on-target DNA binding sites, further supporting the selective stabilization of guide RNA by on-target DNA binding.

### Example 11

### Affinity Tagged gRNA for Detection of Locus-Specific Chromatin Interactions

Experiments conducted during the development of embodiments of the technology described herein indicated that guide RNAs are protected by on-target binding in CRISPR complexes. According, during the development of embodiments of the technology described herein, experiments were conducted in which guide RNA labeled with affinity tags were used to detect locus-specific chromatin interactions. Biotin-labeled guide RNA was introduced into cells using RNP delivery of dCas9/RNA complex or by introducing dCas9-expressing cells via RNA delivery. Streptavidin-coated beads were used to affinity purify chromatin complexes of the genomic targets, and mass spectrometry was used to identify the purified DNA/RNA/protein components within the site-specific chromatin complexes.

Initially, experiments targeted loci at chromosome telomeres using biotin-labeled crRNA:tracrRNA complexes and a non-targeting guide RNA as control. qPCR data were collected, which indicated enrichment of telomere sequences in the isolated chromatin complexes. Moreover, the mass spectrometry data indicated the enrichment of many telomere associated proteins in the purified complexes.

In contrast to previous methods using biotinylated dCas9 (e.g., using biotinylated dCas9 for in situ capture of components interacting with chromatin at a particular locus, e.g., as described in Liu et al. (2017) “In Situ Capture of Chromatin Interactions by Biotinylated dCas9” Cell 170: 1028-43, incorporated herein by reference), embodiments of the technology described herein using biotinylated guide RNA are more specific in detecting locus-specific chromatin interactions. Three populations of dCas9 (on-target binding, off-target binding, and non-binding) are equally stable in the cell. Further, dCas9 accumulates in the nucleolus, while guide RNAs do not accumulate in the nucleus. Consequently, the conventional affinity purification by biotinylated dCas9 enriches off-target chromatin targets and nucleolus-specific proteins in addition to on-target chromatin. In contrast, the data collected during experiments described herein indicated that guide RNAs were selectively protected by binding to on-target DNAs and that unprotected guide RNAs were quickly degraded in cellular environments. Accordingly, affinity purification of chromatin complexes using biotin-labeled RNAs only enriches chromatin complexes for its true genomic targets. As a result, embodiments of the biotinalylated guide RNA methods described herein greatly improve the specificity of affinity purification methods for locus-specific chromatin interactions relative to conventional methods comprising use of biotinylated Cas9 or dCas9.

Moreover, the biotinalylated guide RNAs method also simplifies experimental procedures relative to previous methods using biotinylated dCas9. For example, conventional methods involve producing biotinylated dCas9 in cells and thus require introducing extra components encoding in vivo biotinylation into cells. Importantly, the efficacy of such in vivo biotinylations is difficult to control. In contrast, embodiments of the methods described herein comprise synthesizing biotin-labeled guide RNAs in vitro and directly introducing them into cells via RNP or RNA delivery, which greatly simplifies the experimental procedures.

All publications and patents mentioned in the above specification are herein incorporated by reference in their entirety for all purposes. Various modifications and variations of the described compositions, methods, and uses of the technology will be apparent to those skilled in the art without departing from the scope and spirit of the technology as described. Although the technology has been described in connection with specific exemplary embodiments, it should be understood that the invention as claimed should not be unduly limited to such specific embodiments. Indeed, various modifications of the described modes for carrying out the invention that are obvious to those skilled in the art are intended to be within the scope of the following claims

We claim:

1. A method for imaging a nucleic acid, the method comprising:
(a) contacting a nucleic acid with a detectably labeled ribonucleoprotein (RNP) complex comprising a RNA-guided nuclease and a RNA; and
(b) imaging the nucleic acid by detecting a signal produced by the detectably labeled RNP.

2. The method of claim 1 wherein the detectably labeled RNP comprises a detectably labeled sgRNA, a detectably labeled crRNA, and/or a detectably labeled tracrRNA.

3. The method of claim 1 wherein the detectably labeled RNP comprises a detectably labeled crRNA and the RNP further comprises a tracrRNA.

4. The method of claim 1 wherein the the RNA-guided nuclease comprises a detectably labeled dCas9.

5. The method of claim 1 further comprising producing the RNA-guided nuclease and/or the RNA in vitro.

6. The method of claim 1 further comprising assembling the detectably labeled RNP in vitro from the RNA-guided nuclease and the RNA.

7. The method of claim 1 further comprising delivering the detectably labeled RNP into a cell comprising the nucleic acid.

8. The method of claim 7 wherein the cell is a living cell.

9. The method of claim 7 wherein the cell is a primary cell.

10. The method of claim 1 wherein the nucleic acid is a chromosome or messenger RNA.

11. The method of claim 1 wherein the RNA-guided nuclease is a dCas9, dCpf1, or dCas13.

12. A method of detecting a chromosomal abnormality in a sample, the method comprising:
(a) delivering into a cell a detectably labeled ribonucleoprotein (RNP) complex comprising a RNA-guided nuclease and a RNA comprising a chromosome-specific nucleotide sequence;
(b) acquiring an image of the cell;
(c) counting the number of labeled foci in the image, wherein a number of labeled foci that is abnormal indicates that the sample comprises a chromosomal abnormality.

13. A method of detecting a chromosomal abnormality in a sample, the method comprising:
(a) delivering into a cell a detectably labeled ribonucleoprotein (RNP) complex comprising a RNA-guided nuclease and a RNA comprising a chromosome-specific nucleotide sequence;
(b) acquiring a time-lapse image of the cell;
(c) counting the number of labeled foci in the image; and
(d) comparing the shapes of tracks made by chromosomes in the time-lapse image,
wherein a track having an abnormal shape indicates a false-positive signal to be excluded from counting.

14. A system for imaging a nucleic acid, the system comprising:
(i) a detectably labeled RNP; and
a fluorescence detector.

15. The system of claim 14 further comprising a microscope.

16. The system of claim 14 further comprising a computer programmed to acquire an image, analyze the image to identify labeled foci in the image, count labeled foci, and/or output a result.

17. The system of claim 14 wherein the detectably labeled RNP comprises a detectably labeled sgRNA, a detectably labeled tracrRNA, or a detectably labeled crRNA.

18. The system of claim 14 wherein the detectably labeled RNP comprises a detectably labeled crRNA and further comprises a tracrRNA.

19. The system of claim 14 wherein the detectably labeled RNP comprises a dCas9, a dCpf1, or a Cas13.

20. The system of claim 14 further comprising an input for a sample.

21. The system of claim 14 further comprising a component for introducing the detectably labeled RNP into a cell.

22. A kit for imaging a nucleic acid, the kit comprising a detectably labeled RNP, a detectably labeled RNA-guided nuclease, and/or a detectably labeled RNA.

23. The kit of claim 22 wherein the detectably labeled RNA is a sgRNA, a crRNA, and/or a tracrRNA.

24. The kit of claim 22 wherein the detectably labeled RNA is a crRNA and the kit further comprises a tracrRNA.

25. The kit of claim 22 comprising a dCas9, dCpf1, or Cas13.

26. Use of a RNP complex comprising a detectably labeled RNP to image a nucleic acid.

27. The use of claim 26 wherein the detectably labeled RNP comprises a detectably labeled sgRNA, a detectably labeled crRNA, and/or a detectably labeled tracrRNA.

28. The use of claim 26 wherein the detectably labeled RNP comprises a detectably labeled crRNA and further comprises a tracrRNA.

29. The use of claim 26 wherein a cell comprises the nucleic acid.

30. The use of claim 29 wherein the cell is a living cell.

31. The use of claim 29 wherein the cell is a primary cell.

32. The use of claim 26 wherein the nucleic acid is a chromosome or messenger RNA.

33. The use of claim 26 to detect a chromosomal abnormality.

34. The use of claim 26 to detect an aneuploidy.

35. The use of claim 26 to measure RNA expression.

36. The use of claim 26 to measure RNA localization.

37. The use of claim 26 wherein the detectably labeled RNP comprises a RNA-guided nuclease.

38. The use of claim 37 wherein the RNA-guided nuclease is a dCas9, a dCpf1, or a Cas13.

39. A composition comprising a detectably labeled RNP complex.

40. The composition of claim 39 wherein the detectably labeled RNP complex comprises a detectably labeled sgRNA, a detectably labeled crRNA, and/or a detectably labeled tracrRNA.

41. The composition of claim 39 wherein the detectably labeled RNP complex comprises a detectably labeled crRNA and further comprises a tracrRNA.

42. The composition of claim 39 wherein the detectably labeled RNP complex comprises a fluorescent label or a chemical linker.

43. The composition of claim 39 wherein the detectably labeled RNP complex comprises an RNA comprising a targeting sequence complementary to a chromosomal locus or a messenger RNA.

44. The composition of claim 39 wherein the detectably labeled RNP complex comprises a RNA-guided nuclease.

45. The composition of claim 44 wherein the RNA-guided nuclease is dCas9, dCpf1, or Cas13.

46. A system for detecting locus-specific chromatin interactions, the system comprising:
a) a gRNA comprising a first member of an interacting pair; and
b) a component comprising a second member of said interacting pair.

47. The system of claim 46 further comprising a RNA-guided nuclease.

48. The system of claim 47 wherein the RNA-guided nuclease is dCas9, Cpf1, or Cas13.

49. The system of claim 46 wherein said first member is biotin.

50. The system of claim 46 wherein said second member is avidin or streptavidin.

51. The system of claim 46 wherein said component comprising said second member of said interacting pair comprises a bead.

52. The system of claim 46 wherein said interacting pair comprises an affinity tag and a component specific for the affinity tag.

53. The system of claim 46 wherein said interacting pair comprises an antibody and epitope, a first click chemistry moiety and a second click chemistry moiety, a chelator and a component chelated by the chelator, a first and second component attracted by magnetism, or an aptamer and aptamer ligand.

54. The system of claim 46 wherein the first member of said interacting pair is specific for the second member of said interacting pair.

55. The system of claim 46 wherein the first member of said interacting pair binds to the second member of said interacting pair with a K_{d} of approximately 10⁻⁹ to10⁻¹² M or stronger.

56. A method for detecting expression and localization of a target RNA in a sample, the method comprising:
(a) delivering into a cell a detectably labeled ribonucleoprotein (RNP) complex comprising a RNA-guided nuclease and a RNA comprising a sequence complementary to the target RNA;
(b) acquiring an image of the cell; and
(c) counting the number of labeled foci and quantifying fluorescent intensity in the image.

57. A method of detecting expression and localization of a target RNA in a sample, the method comprising:
(a) delivering into a cell a detectably labeled ribonucleoprotein (RNP) complex comprising a RNA-guided nuclease and a RNA comprising a sequence complementary to the target RNA;
(b) acquiring a time-lapse image of the cell;
(c) counting the number of labeled foci and quantifying fluorescent intensity in the image; and
(d) comparing the shapes of tracks made by labeled loci in the time-lapse image.

58. The method of claim 56 wherein the target RNA is a messenger RNA or non-coding RNA.

59. The method of claim 57 wherein the target RNA is a messenger RNA or non-coding RNA.
